SEQUENCE LISTING (55 sequences)

SEQ ID NO: 1 - protein, 738 aa, synthetic construct
  REGION 1..738: note = VP1
MAADGYLPDWLEDNLSEGIREWWDLKPGAPKPKANQQKQDNGRGLVLPGYKYLGPFNGLDKGEPVNAADAAALEHDKAYDQQLQAGDNPYLRYNHADAEFQERLQEDTSFGGNLGRAVFQAKKRVLEPLGLVESPVKTAPGKKRPVEPSPQRSPDSSTGIGKKGQQPAKKRLNFGQTGDSESVPDPQPIGEPPAAPSGVGPNTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYNFEDVPFHSSYAHSQSLDRLMNPLIDQYLYYLSRTQSTGGTAGTQQLLFSQAGPNNMSAQAKNWLPGPCYRQQRVSTTLSQNNNSNFAWTGATKYHLNGRDSLVNPGVAMATHKDDEERFFPSSGVLMFGKQGAGKDNVDYSSVMLTSEEEIKTTNPVATEQYGVVADNLQQQNAAPIVGAVNSQGALPGMVWQNRDVYLQGPIWAKIPHTDGNFHPSPLMGGFGLKHPPPQILIKNTPVPADPPTTFNQAKLASFITQYSTGQVSVEIEWELQKENSKRWNPEIQYTSNYYKSTNVDFAVNTEGTYSEPRPIGTRYLTRNL

SEQ ID NO: 2 - protein, 736 aa, synthetic construct
  REGION 1..736: note = VP1
MAADGYLPDWLEDNLSEGIREWWALQPGAPKPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVDQSPQEPDSSSGVGKSGKQPARKRLNFGQTGDSESVPDPQPLGEPPAAPTSLGSNTMASGGGAPMADNNEGADGVGNSSGNWHCDSQWLGDRVITTSTRTWALPTYNNHLYKQISSQSGASNDNHYFGYSTPWGYFDFNRFHCHFSPRDWQRLINNNWGFRPKKLSFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNRTQGTTSGTTNQSRLLFSQAGPQSMSLQARNWLPGPCYRQQRLSKTANDNNNSNFPWTAASKYHLNGRDSLVNPGPAMASHKDDEEKFFPMHGNLIFGKEGTTASNAELDNVMITDEEEIRTTNPVATEQYGTVANNLQSSNTAPTTRTVNDQGALPGMVWQDRDVYLQGPIWAKIPHTDGHFHPSPLMGGFGLKHPPPQIMIKNTPVPANPPTTFSPAKFASFITQYSTGQVSVEIEWELQKENSKRWNPEIQYTSNYNKSVNVDFTVDTNGVYSEPRPIGTRYLTRPL

SEQ ID NO: 3 - DNA, 1574 bp, Homo sapiens
  source 1..1574: mol_type = other DNA
gggcggggcgggaggcggggcccggcccgttaagaagagcgtggccggccgcggccaccgctggccccagggaaagccgagcggccaccgagccggcagagacccaccgagcggcggcggagggagcagcgccggggcgcacgagggcaccatggcccagacgcccgccttcgacaagcccaaagtagaactgcatgtccacctagacggatccatcaagcctgaaaccatcttatactatggcaggaggagagggatcgccctcccagctaacacagcagaggggctgctgaacgtcattggcatggacaagccgctcacccttccagacttcctggccaagtttgactactacatgcctgctatcgcgggctgccgggaggctatcaaaaggatcgcctatgagtttgtagagatgaaggccaaagagggcgtggtgtatgtggaggtgcggtacagtccgcacctgctggccaactccaaagtggagccaatcccctggaaccaggctgaaggggacctcaccccagacgaggtggtggccctagtgggccagggcctgcaggagggggagcgagacttcggggtcaaggcccggtccatcctgtgctgcatgcgccaccagcccaactggtcccccaaggtggtggagctgtgtaagaagtaccagcagcagaccgtggtagccattgacctggctggagatgagaccatcccaggaagcagcctcttgcctggacatgtccaggcctaccaggaggctgtgaagagcggcattcaccgtactgtccacgccggggaggtgggctcggccgaagtagtaaaagaggctgtggacatactcaagacagagcggctgggacacggctaccacaccctggaagaccaggccctttataacaggctgcggcaggaaaacatgcacttcgagatctgcccctggtccagctacctcactggtgcctggaagccggacacggagcatgcagtcattcggctcaaaaatgaccaggctaactactcgctcaacacagatgacccgctcatcttcaagtccaccctggacactgattaccagatgaccaaacgggacatgggctttactgaagaggagtttaaaaggctgaacatcaatgcggccaaatctagtttcctcccagaagatgaaaagagggagcttctcgacctgctctataaagcctatgggatgccaccttcagcctctgcagggcagaacctctgaagacgccactcctccaagccttcaccctgtggagtcaccccaactctgtggggctgagcaacatttttacatttattccttccaagaagaccatgatctcaatagtcagttactgatgctcctgaaccctatgtgtccatttctgcacacacgtatacctcggcatggccgcgtcacttctctgattatgtgccctggccagggaccagcgcccttgcacatgggcatggttgaatctgaaaccctccttctgtggcaacttgtactgaaaatctggtgctcaataaagaagcccatggctggtggcatgcagcaaaaaaaaaaaaaaaaa

SEQ ID NO: 4 - protein, 363 aa, Homo sapiens
MAQTPAFDKPKVELHVHLDGSIKPETILYYGRRRGIALPANTAEGLLNVIGMDKPLTLPDFLAKFDYYMPAIAGCREAIKRIAYEFVEMKAKEGVVYVEVRYSPHLLANSKVEPIPWNQAEGDLTPDEVVALVGQGLQEGERDFGVKARSILCCMRHQPNWSPKVVELCKKYQQQTVVAIDLAGDETIPGSSLLPGHVQAYQEAVKSGIHRTVHAGEVGSAEVVKEAVDILKTERLGHGYHTLEDQALYNRLRQENMHFEICPWSSYLTGAWKPDTEHAVIRLKNDQANYSLNTDDPLIFKSTLDTDYQMTKRDMGFTEEEFKRLNINAAKSSFLPEDEKRELLDLLYKAYGMPPSASAGQNL

SEQ ID NO: 5 - DNA, 4510 bp, Homo sapiens
  source 1..4510: mol_type = other DNA
aaactcagatgcacacagcaagcatgcatgtaagccacacacttgccactctggctgcctcagcactgcagagcccctctccccggcctctagttggatccctggtgcacactggagggaaataaaacctaccccaacaggaagtgaaacagttggtgagcttttccggtgctctgcacagatgctggggcgctgagcaaacagccctcagtttctggagctgttccgagtcccgtggagtctccatctgagccctttcctagtccaggcatcccgatgttggtggatggcccatctgagcggccagccctgtgcttcttgctgttggctgtggcaatgtctttcttcggctcagctctatccatagatgaaacacgggcgcatctgttgttgaaagaaaagatgatgcggctgggggggcggctggtgctgaacaccaaggaggagctggccaatgagaggctcatgacgctcaaaatcgctgagatgaaggaggccatgaggaccctgatattcccacccagcatgcactttttccaggccaagcatctcattgagagaagtcaagtgtttaatattctaaggatgatgccaaaaggggctgccttgcacctccatgacattggcatcgtgactatggactggctggtgaggaatgtcacctacaggcctcactgccacatctgtttcaccccaagggggatcatgcagttcagatttgctcacccaactccccgtccatcagaaaaatgttccaagtggattctgctggaggattatcggaagcgggtgcagaacgtcactgagtttgatgacagcttgctgaggaatttcactctggtgacccagcacccggaggtgatttacacaaaccaaaatgttgtctggtcgaaatttgaaaccatcttcttcaccatctctggtctcatccattacgcaccagtgttcagagactatgtcttccggagcatgcaggagttctacgaggacaacgtgctctacatggagatcagagccaggctgctgccggtgtatgagctcagtggagagcaccatgacgaagagtggtcagtgaagacttaccaggaagtagctcagaagtttgtggaaactcaccctgagtttattggaatcaaaatcatttattcggatcacagatccaaagatgtggctgtcatcgcagaatccatccgaatggccatggggctccgaatcaagttccccacggtggtggcagggtttgacctggtggggcatgaggacactggccactccttgcatgactacaaggaagctctgatgatccccgccaaggatggcgttaagctgccttacttcttccacgccggagaaacagactggcagggtacttccatagacaggaacattctggatgctctgatgctgaacactaccagaatcggccatggatttgctttgagcaaacaccccgcagtcaggacttactcctggaaaaaggacatccccatagaagtctgtcccatctctaaccaggtgctgaaactggtgtctgacttgaggaaccaccctgtagccactctgatggccactgggcaccccatggtgatcagctctgatgacccagctatgtttggtgccaaaggcttgtcctatgatttctatgaggtcttcatgggcattggggggatgaaggctgacctgaggaccctcaaacagctggccatgaactctatcaagtacagtaccctgttggagagtgagaaaaatactttcatggaaatctggaagaagagatgggataagttcatagcagatgtggctacaaagtgaggagaagctagccagccctctacaagctgtcttcttgcacacgctgtcacttcctctcactcgttcttgaatcagctccatgtgcccatgaaatcaatggcctctgtatggagcgaccctgtgagaagcacttggctggctgagcaaattcatcctctggaaatattctctctcagccacagtgacattgaccctcttggttttctcctgtctctggccatttcttccagtttccctatttcagagtcttctcctctctctgatctctgtgctgtttcctcaggactcagtcctgggctctcttctattctggtctctttatttttttatttttgtattttttcgagatggagttttgctcttgttgcccaggctggagtacaatggtgcgatctcagctcagtgcaacctccgccacccgggttcaggcaattctcttgcatcagcctcgcgagtagttggaattataggcatgtgccaccacacccagctgatttttgcatttttagtagagacaggttttcaccatgttggcgaggctggtatccaactcttgacctcaggtgatccactcgccccttggctcccaaagtgctggaattacaggcattagccaccatgcctggcctattctggtctctttaactctctcctctttatttctcttctctctctgtacacttttcctgggtggtctcatccattcctttgctttttcataccatttatttgttaatgattcccacatttatttatgcacttggagagctcacaggaatctcagaaactgatgaggtacaattctgaaccctcagtctcttccctttaaacctttctttttctctactttaatttttctaaagagtgtcttgctatgttgcccaggctggtctccaactcaagtgatcctcctgccgcagtctcccgaagtgctgggattactgacatgagccaccacactcagccctttaaacctttccctggcctttcccatagctggtgaaggacacctccatccattccacgcagttgctcaaagcagaaattttcagtgcaagtcttgatgctgcgccgtcccccactccctacatcagaacgcatccctcatctggactccagcggtggcttcttgatgctgcgcggtcccccactccctacatcagaatgcatcccgcatccagactccagcggtggtgctctacctgcacgctgttgccaagtccaagctaccatactcctgcctgagctatgacaacagcctcctcactgatctcccctttcttccctttgcctcctccagctcatttttcacagtgtagaatgacattttgtttgtttgttttgttttgtttgagatggagtctcgctctgttgcccagggtggagtgcagcggtgcgatctcggctcactgcaacctccacctcccgggttcaagcggattctcgtgcctcagcctcctgagtagctgggattacaggcatgcaccaccatgcccggataatttttgtatttttagtagagatggggtttcactatgttggccaggctggtctcgaacacctgacctcgtggtccacccgcctcggcctcccaaagcactgggattacaggcgtgagccacccggcctggcctagaatgacttttaaaagatcaaattaaatcaggtcactcctttgcttacaacgcagtgcgtttagaggtacaccccatgtctccacagggcatacagcatccgatttaatctggatccattccggcgccttcctctcccagtcacccagagggccccaaccccggcggccctttcttcctcaaatgtcctcggctctataccgtgcctgggtcttttctctttctctctgcctggaagattccttctttccccttttgtcttgcccactcctgtttacccttcaagtttcaagttcatgtcactgtctcagagaggttttcctgtgctcgccctgtttctctcaggaagccttgctcttttccatcatgcctctaatcacagcttataatcggatatttatttctgtgtctacagtcttgccctgccagactgtatgccccatgtgggcaggcgctcatgattgtttctgattgtttcacgcatgctgctaacccagagcctgggcccaaagctagttagtactcaataaacaatgcattgaatgagtggctgtcactgtgtctgttcagccagctgccaaggcagaggggagtagagcagagccgccccaaataaaagacctcatgtgatcagagtccagctcctcacctggccttggaagaaggatgcaagaagccacctctgtcctcacccaactcaaggatggcagggaatcaaagaattcctcaaaagccccaggcgaatgtcctctcatccatcttccatgacctgtacctgagctctcagacagcagcaagccctctctccattcctagtttatcaccatttggttcctggaactgggaaaaaacagcaaccataagctgtgcctggatggaagaagacaacctctagggtcctgtctaactctgcaggtgattcagtgccttacccaatccctgaagatcttttaggtcccaactgcgcaggggacctccaacacaggcagacgcaaacagcctctggcctggacttgctgtgatctctgatttggcaaaatgaaccagcaatagtcttgcaaaaa

SEQ ID NO: 6 - protein, 511 aa, Homo sapiens
MLVDGPSERPALCFLLLAVAMSFFGSALSIDETRAHLLLKEKMMRLGGRLVLNTKEELANERLMTLKIAEMKEAMRTLIFPPSMHFFQAKHLIERSQVFNILRMMPKGAALHLHDIGIVTMDWLVRNVTYRPHCHICFTPRGIMQFRFAHPTPRPSEKCSKWILLEDYRKRVQNVTEFDDSLLRNFTLVTQHPEVIYTNQNVVWSKFETIFFTISGLIHYAPVFRDYVFRSMQEFYEDNVLYMEIRARLLPVYELSGEHHDEEWSVKTYQEVAQKFVETHPEFIGIKIIYSDHRSKDVAVIAESIRMAMGLRIKFPTVVAGFDLVGHEDTGHSLHDYKEALMIPAKDGVKLPYFFHAGETDWQGTSIDRNILDALMLNTTRIGHGFALSKHPAVRTYSWKKDIPIEVCPISNQVLKLVSDLRNHPVATLMATGHPMVISSDDPAMFGAKGLSYDFYEVFMGIGGMKADLRTLKQLAMNSIKYSTLLESEKNTFMEIWKKRWDKFIADVATK

SEQ ID NO: 7 - DNA, 6692 bp, Homo sapiens
  source 1..6692: mol_type = other DNA
gcccctcctcttggccaaactttccggaggggaaggctttccgaggaaacgaaagcgaaattgaaccggagccatcttgggcccggcgcgcagacccgcggagtttcccgtgccgacgccccggggccacttccagtgcggagtagcggaggcgtgggggcctcgaggggctggcgcggcccagcggtcgggccagggtcgtgccgccggcgggtcgggccgggcaatgcctcgcgggcgcaatgaatccgcggcaggggtattccctcagcggatactacacccatccatttcaaggctatgagcacagacagctcaggtaccagcagcctgggccaggatcttcccccagtagtttcctgcttaagcaaatagaatttctcaaggggcagctcccagaagcaccggtgattggaaagcagacaccgtcactgccaccttccctcccaggactccggccaaggtttccagtactacttgcctccagtaccagaggcaggcaagtggacatcaggggtgtccccaggggcgtgcatctcggaagtcaggggctccagagagggttccagcatccttcaccacgtggcaggagtctgccacagagaggtgttgattgcctttcctcacatttccaggaactgagtatctaccaagatcaggaacaaaggatcttaaagttcctggaagagcttggggaagggaaggccaccacagcacatgatctgtctgggaaacttgggactccgaagaaagaaatcaatcgagttttatactccctggcaaagaagggcaagctacagaaagaggcaggaacaccccctttgtggaaaatcgcggtctccactcaggcttggaaccagcacagcggagtggtaagaccagacggtcatagccaaggagccccaaactcagacccgagtttggaaccggaagacagaaactccacatctgtctcagaagatcttcttgagccttttattgcagtctcagctcaggcttggaaccagcacagcggagtggtaagaccagacagtcatagccaaggatccccaaactcagacccaggtttggaacctgaagacagcaactccacatctgccttggaagatcctcttgagtttttagacatggccgagatcaaggagaaaatctgcgactatctcttcaatgtgtctgactcctctgccctgaatttggctaaaaatattggccttaccaaggcccgagatataaatgctgtgctaattgacatggaaaggcagggggatgtctatagacaagggacaacccctcccatatggcatttgacagacaagaagcgagagaggatgcaaatcaagagaaatacgaacagtgttcctgaaaccgctccagctgcaatccctgagaccaaaagaaacgcagagttcctcacctgtaatatacccacatcaaatgcctcaaataacatggtaaccacagaaaaagtggagaatgggcaggaacctgtcataaagttagaaaacaggcaagaggccagaccagaaccagcaagactgaaaccacctgttcattacaatggcccctcaaaagcagggtatgttgactttgaaaatggccagtgggccacagatgacatcccagatgacttgaatagtatccgcgcagcaccaggtgagtttcgagccatcatggagatgccctccttctacagtcatggcttgccacggtgttcaccctacaagaaactgacagagtgccagctgaagaaccccatcagcgggctgttagaatatgcccagttcgctagtcaaacctgtgagttcaacatgatagagcagagtggaccaccccatgaacctcgatttaaattccaggttgtcatcaatggccgagagtttcccccagctgaagctggaagcaagaaagtggccaagcaggatgcagctatgaaagccatgacaattctgctagaggaagccaaagccaaggacagtggaaaatcagaagaatcatcccactattccacagagaaagaatcagagaagactgcagagtcccagacccccaccccttcagccacatccttcttttctgggaagagccccgtcaccacactgcttgagtgtatgcacaaattggggaactcctgcgaattccgtctcctgtccaaagaaggccctgcccatgaacccaagttccaatactgtgttgcagtgggagcccaaactttccccagtgtgagtgctcccagcaagaaagtggcaaagcagatggccgcagaggaagccatgaaggccctgcatggggaggcgaccaactccatggcttctgataaccagcctgaaggtatgatctcagagtcacttgataacttggaatccatgatgcccaacaaggtcaggaagattggcgagctcgtgagatacctgaacaccaaccctgtgggtggccttttggagtacgcccgctcccatggctttgctgctgaattcaagttggtcgaccagtccggacctcctcacgagcccaagttcgtttaccaagcaaaagttgggggtcgctggttcccagccgtctgcgcacacagcaagaagcaaggcaagcaggaagcagcagatgcggctctccgtgtcttgattggggagaacgagaaggcagaacgcatgggtttcacagaggtaaccccagtgacaggggccagtctcagaagaactatgctcctcctctcaaggtccccagaagcacagccaaagacactccctctcactggcagcaccttccatgaccagatagccatgctgagccaccggtgcttcaacactctgactaacagcttccagccctccttgctcggccgcaagattctggccgccatcattatgaaaaaagactctgaggacatgggtgtcgtcgtcagcttgggaacagggaatcgctgtgtgaaaggagattctctcagcctaaaaggagaaactgtcaatgactgccatgcagaaataatctcccggagaggcttcatcaggtttctctacagtgagttaatgaaatacaactcccagactgcgaaggatagtatatttgaacctgctaagggaggagaaaagctccaaataaaaaagactgtgtcattccatctgtatatcagcactgctccgtgtggagatggcgccctctttgacaagtcctgcagcgaccgtgctatggaaagcacagaatcccgccactaccctgtcttcgagaatcccaaacaaggaaagctccgcaccaaggtggagaacggagaaggcacaatccctgtggaatccagtgacattgtgcctacgtgggatggcattcggctcggggagagactccgtaccatgtcctgtagtgacaaaatcctacgctggaacgtgctgggcctgcaaggggcactgttgacccacttcctgcagcccatttatctcaaatctgtcacattgggttaccttttcagccaagggcatctgacccgtgctatttgctgtcgtgtgacaagagatgggagtgcatttgaggatggactacgacatccctttattgtcaaccaccccaaggttggcagagtcagcatatatgattccaaaaggcaatccgggaagactaaggagacaagcgtcaactggtgtctggctgatggctatgacctggagatcctggacggtaccagaggcactgtggatgggccacggaatgaattgtcccgggtctccaaaaagaacatttttcttctatttaagaagctctgctccttccgttaccgcagggatctactgagactctcctatggtgaggccaagaaagctgcccgtgactacgagacggccaagaactacttcaaaaaaggcctgaaggatatgggctatgggaactggattagcaaaccccaggaggaaaagaacttttatctctgcccagtatagtatgctccagtgacagatggattagggtgtgtcatactagggtgtgagagaggtaggtcgtagcattcctcatcacatggtcaggggatttttttttctcctttttttttctttttaagccataattggtgatactgaaaactttgggttcccatttatcctgctttctttgggattgctaggcaaggtctggccaggccccccttttttcccccaagtgaagaggcagaaacctaagaagttatcttttctttctacccaaagcatacatagtcactgagcacctgcggtccatttcctcttaaaagttttgttttgatttgtttccatttcctttccctttgtgtttgctacactgacctcttgcggtcttgattaggtttcagtcaactctggatcatgtcagggactgataatttcatttgtggattacgcagacccctctacttcccctctttcccttctgagattctttccttgtgatctgaatgtctccttttccccctcagagggcaaagaggtgaacataaaggatttggtgaaacatttgtaagggtaggagttgaaaactgcagttcccagtgccacggaagtgtgattggagcctgcagataatgcccagccatcctcccatcctgcactttagccagctgcagggcgggcaaggcaaggaaagctgcttccctggaagtgtatcactttctccggcagctgggaagtctagaaccagccagactgggttaagggagctgctcaagcaatagcagaggtttcacccggcaggatgacacagaccacttcccagggagcacgggcatgccttggaatattgccaagcttccagctgcctcttctcctaaagcattcctaggaatattttccccgccaatgctgggcgtacaccctagccaacgggacaaatcctagagggtataaaatcatctctgctcagataatcatgacttagcaagaataagggcaaaaaatcctgttggcttaacgtcactgttccacccggtgtaatatctctcatgacagtgacaccaagggaagttgactaagtcacatgtaaattaggagtgttttaaagaatgccatagatgttgattcttaactgctacagataacctgtaattgagcagatttaaaattcaggcatacttttccatttatccaagtgctttcatttttccagatggcttcagaagtaggctcgtgggcagggcgcagacctgatctttatagggttgacatagaaagcagtagttgtgggtgaaagggcaggttgtcttcaaactctgtgaggtagaatcctttgtctatacctccatgaacattgactcgtgtgttcagagcctttggcctctctgtggagtctggctctctggctcctgtgcattctttgaatagtcactcgtaaaaactgtcagtgcttgaaactgtttcctttactcatgttgaagggactttgttggcttttagagtgttggtcatgactccaagagcagagcagggaagagcccaagcatagacttggtgccgtggtgatggctgcagtccagttttgtgatgctgcttttacgtgtccctcgataacagtcagctagacacactcaggaggactactgaggctctgcgaccttcaggagctgagcctgcctctctcctttagatgacagaccttcatctgggaacgtgctgagccagcaccctcagatgatttccctccaaactgctgactaggtcatcctctgtctggtagagacattcacatctttgcttttattctatgctctctgtacttttgaccaaaaattgaccaaagtaagaaaatgcaagttctaaaaatagactaaggatgcctttgcagaacaccaaagcatcccaaggaactggtagggaagtggcgcctgtctcctggagtggaagaggcctgctccctggctctgggtctgctgggggcacagtaaatcagtcttggcacccacatccagggcagagaggtctgtggttctcagcatcagaaggcagcgcagcccctctcctcttcaggctacagggttgtcacctgctgagtcctcaggttgtttggcctctctggtccatcttgggcattaggttctccagcagagctctggccagctgcctcttctttaactgggaacacaggctctcacaagatcagaacccccactcacccccaagatcttatctagcaagcctgtagtattcagtttctgttgtaggaagagagcgaggcatccctgaattccacgcatctgctggaaacgagccgtgtcagatcgcacatccctgcgcccccatgcccctctgagtcacacaggacagaggaggcagagcttctgcccactgttatcttcactttctttgtccagtcttttgtttttaataagcagtgaccctccctactcttctttttaatgatttttgtagttgatttgtctgaactgtggctactgtgcattccttgaataatcacttgtaaaaattgtcagtgcttgaagctgtttcctttactcacattgaagggacttcgttggttttttggagtcttggttgtgactccaagagcagagtgaggaagacccccaagcatagactcgggtactgtgatgatggctgcagtccagttttatgattctgcttttatgtgtcccttgataacagtgacttaacaatatacattcctcataaataaaaaaaaaacaagaatctgaattcttagaaaaaaaaaaaaaaaaaaaaaaa

SEQ ID NO: 8 - protein, 1226 aa, Homo sapiens
MNPRQGYSLSGYYTHPFQGYEHRQLRYQQPGPGSSPSSFLLKQIEFLKGQLPEAPVIGKQTPSLPPSLPGLRPRFPVLLASSTRGRQVDIRGVPRGVHLGSQGLQRGFQHPSPRGRSLPQRGVDCLSSHFQELSIYQDQEQRILKFLEELGEGKATTAHDLSGKLGTPKKEINRVLYSLAKKGKLQKEAGTPPLWKIAVSTQAWNQHSGVVRPDGHSQGAPNSDPSLEPEDRNSTSVSEDLLEPFIAVSAQAWNQHSGVVRPDSHSQGSPNSDPGLEPEDSNSTSALEDPLEFLDMAEIKEKICDYLFNVSDSSALNLAKNIGLTKARDINAVLIDMERQGDVYRQGTTPPIWHLTDKKRERMQIKRNTNSVPETAPAAIPETKDNAEFLTCNIPTSNASNNMVTTEKVENGQEPVIKLENRQEARPEPARLKPPVHYNGPSKAGYVDFENGQWATDDIPDDLNSIRAAPGEFRAIMEMPSFYSHGLPRCSPYKKLTECQLKNPISGLLEYAQFASQTCEFNMIEQSGPPHEPRFKFQVVINGREFPPAEAGSKKVAKQDAAMKAMTILLEEAKAKDSGKSEESSHYSTEKESEKTAESQTPTPSATSFFSGKSPVTTLLECMHKLGNSCEFRLLSKEGPAHEPKFQYCVAVGAQTFPSVSAPSKKVAKQMAAEEAMKALHGEATNSMASDNQPEGMISESLDNLESMMPNKVRKIGELVRYLNTNPVGGLLEYARSHGFAAEFKLVDQSGPPHEPKFVYQAKVGGRWFPAVCAHSKKQGKQEAADAALRVLIGENEKAERMGFTEVTPVTGASLRRTMLLLSRSPEAQPKTLPLTGSTFHDQIAMLSHRCFNTLTNSFQPSLLGRKILAAIIMKKDSEDMGVVVSLGTGNRCVKGDSLSLKGETVNDCHAEIISRRGFIRFLYSELMKYNSQTAKDSIFEPAKGGEKLQIKKTVSFHLYISTAPCGDGALFDKSCSDRAMESTESRHYPVFENPKQGKLRTKVENGEGTIPVESSDIVPTWDGIRLGERLRTMSCSDKILRWNVLGLQGALLTHFLQPIYLKSVTLGYLFSQGHLTRAICCRVTRDGSAFEDGLRHPFIVNHPKVGRVSIYDSKRQSGKTKETSVNWCLADGYDLEILDGTRGTVDGPRNELSRVSKKNIFLLFKKLCSFRYRRDLLRLSYGEAKKAARDYETAKNYFKKGLKDMGYGNWISKPQEEKNFYLCPV

SEQ ID NO: 9 - DNA, 6924 bp, Homo sapiens
  source 1..6924: mol_type = other DNA
ctgaggcgctgaggcggccgtggcggcggcggcggcggcggcggcagcggcggccaagcggccaggttggcggccggggctccgggccgcgcgaggccacggccacgccgcgccgctgcgcacaaccaacgaggcagagcgccgcccggcgcgagactgcggccgaagcgtggggcgcgcgtgcggaggaccaggcgcggcgcggctgcggctgagagtggagcctttcaggctggcatggagagcttaaggggcaactgaaggagacacactggccaagcgcggagttctgcttacttcagtcctgctgagatactctctcagtccgctcgcaccgaaggaagctgccttgggatcagagcagacataaagctagaaaaatttcaagacagaaacagtctccgccagtcaagaaaccctcaaaagtattttgccatggatatagaagatgaagaaaacatgagttccagcagcactgatgtgaaggaaaaccgcaatctggacaacgtgtcccccaaggatggcagcacacctgggcctggcgagggctctcagctctccaatgggggtggtggtggccccggcagaaagcggcccctggaggagggcagcaatggccactccaagtaccgcctgaagaaaaggaggaaaacaccagggcccgtcctccccaagaacgccctgatgcagctgaatgagatcaagcctggtttgcagtacacactcctgtcccagactgggcccgtgcacgcgcctttgtttgtcatgtctgtggaggtgaatggccaggtttttgagggctctggtcccacaaagaaaaaggcaaaactccatgctgctgagaaggccttgaggtctttcgttcagtttcctaatgcctctgaggcccacctggccatggggaggaccctgtctgtcaacacggacttcacatctgaccaggccgacttccctgacacgctcttcaatggttttgaaactcctgacaaggcggagcctcccttttacgtgggctccaatggggatgactccttcagttccagcggggacctcagcttgtctgcttccccggtgcctgccagcctagcccagcctcctctccctgtcttaccaccattcccacccccgagtgggaagaatcccgtgatgatcttgaacgaactgcgcccaggactcaagtatgacttcctctccgagagcggggagagccatgccaagagcttcgtcatgtctgtggtcgtggatggtcagttctttgaaggctcggggagaaacaagaagcttgccaaggcccgggctgcgcagtctgccctggccgccatttttaacttgcacttggatcagacgccatctcgccagcctattcccagtgagggtcttcagctgcatttaccgcaggttttagctgacgctgtctcacgcctggtcctgggtaagtttggtgacctgaccgacaacttctcctcccctcacgctcgcagaaaagtgctggctggagtcgtcatgacaacaggcacagatgttaaagatgccaaggtgataagtgtttctacaggaacaaaatgtattaatggtgaatacatgagtgatcgtggccttgcattaaatgactgccatgcagaaataatatctcggagatccttgctcagatttctttatacacaacttgagctttacttaaataacaaagatgatcaaaaaagatccatctttcagaaatcagagcgaggggggtttaggctgaaggagaatgtccagtttcatctgtacatcagcacctctccctgtggagatgccagaatcttctcaccacatgagccaatcctggaagaaccagcagatagacacccaaatcgtaaagcaagaggacagctacggaccaaaatagagtctggtgaggggacgattccagtgcgctccaatgcgagcatccaaacgtgggacggggtgctgcaaggggagcggctgctcaccatgtcctgcagtgacaagattgcacgctggaacgtggtgggcatccagggatccctgctcagcattttcgtggagcccatttacttctcgagcatcatcctgggcagcctttaccacggggaccacctttccagggccatgtaccagcggatctccaacatagaggacctgccacctctctacaccctcaacaagcctttgctcagtggcatcagcaatgcagaagcacggcagccagggaaggcccccaacttcagtgtcaactggacggtaggcgactccgctattgaggtcatcaacgccacgactgggaaggatgagctgggccgcgcgtcccgcctgtgtaagcacgcgttgtactgtcgctggatgcgtgtgcacggcaaggttccctcccacttactacgctccaagattaccaagcccaacgtgtaccatgagtccaagctggcggcaaaggagtaccaggccgccaaggcgcgtctgttcacagccttcatcaaggcggggctgggggcctgggtggagaagcccaccgagcaggaccagttctcactcacgccctgacccgggcagacatgatggggggtgcagggggctgtgggcatccagcgtcatcctccagaacctcacatctgaactgggggcaggtgcataccttggggagggagtagggggacacgggggaccaccaggtgtccacggttgtccccagcatctcacatcagacctggggcaggtgcgcagtgtggggaggggatggggtgcgtcagggcccagcatcgccgcctggcatctctctgccgcagcatttccccttctgaaccgtccagtgactgctttcaatctcggtttacgtttagaaattgagttctactgagtagggcttccttaagtttaggaaaatagaaattactttgtgtgaaattcttgaataaataatttattcagagctaggaatgtggtttataaaataggaagtaattgtgtcaggtcacttttatgccacattattttaattgcaaaaaagcatctatatatggaggagggtgggaaaatagaggtaggaaatagtagcctaaaggaaatcgccacacgtctgtctaaacttaggtctcttttctccgtaggtacctccctgggtagttccacacactaggttgtaacagtctctccctgaggagcagactcccagcatggtgtagcgtggccctgtcatgcacatggggtcccgcagcagtgactgtgtgtcctgcagaggcgtgacccaggcccctgtagccctcagcctcctctagaagcttctgtactccttgtaggatcagatcatggaaaacttttctcagtttacttctaagtaatcacagataatacatggccagtaatcccaggctggccattcattcaggttttttaaaggatatttaacttttatggactagaaggaatcacgagggctactgcacaatacatggcctaagttccctctgttccttcctctgaatcgaatggatgtgggtgaccgcccgaaggccttcacaggatggaagtagaatgatttcagtagatactcattcttggaaaatgccatagttttaaattattgtttccagctttatcaaagacatgtttgaaaaataaaaagcatccaagtgagagctggtgagaccacgtgctgctggcgtagtgtaggccagacattgacagtcctgacgggagctcagggctgcccagcgcccagcgtgcacgggacggccccacgacagagggagtcagcccgggaggtcaggagcgcggcgggcgagggccctgtgtggaccacctccaccaagctcagagatttgcaccaggtgccttgttgcctccgctcaggatgaaagaggagctgagagaagtgctctgcctgccagtgcagtgcccagctccaaggctctagagggtgttcaggtgggtctcctggggccatggggagagattggtgcagaccttaccccacagcatacacctgccacagcgaaatccagggtgttggcacctgtgtgtccgtgatgagcctaggaaaccagagcaggggcagaggggcgtcatcctcccaccggacgctgggagctcagaccccaaaactgaaacaccgtggcttcggcggggggtgtgcctcctgatgtcaggagccccatccacgtgtgtccacacagatctcgtcgcagcacggcaggaaggggtgctgcttagggctcattgttggggacatgaccgggttcagcggctagaacatctgccccacagcagcctcctcctccaccgaagagggtagttgtctccctgaagcagtcacagcaggcgtctctgccgctccgtcaccacagtggggttttgttcaggcagatcgcgctggggttctgcacctgcagaaggagaggggtctgttgtcgctggctttcccccaagcaggctcttgcacactctagaaaaaacaccttgtaagtctgtgcatttttattgtcttgataaattgtatttttttctaatggggattgggagatggacttcgtttttaaaaatatgtggattttggttaccaagtttagtgttaatatattccatatacatacaaaactacccggtatgtctggcttttcccttctgtcaggtaatagctaaagtcagcatgattgctccctgtaccaccccaaataagtgagtgcctcaccttgtggggcctgagcagctaccttgagaccatgtgaggtggcacctttccggggtggactcgtgcggccttgaggacaggcacagggcaccctatcccaagccgtccaggcaggaggaaggcagccaaggcaactgggttctgggagccctgggtggggcagctgtggggaggaactgggttcggggagccctgggcggggcggctgttggggggaactgggttcggggtgccctgggcagggggctactggggggcggctgtgaggaggagttgggttcagggagccctgggcggggtggctgtcagggggaactgggttccgggagccctgggccggggcagggggcggctgtaggaaggaactggtttcggggagccctgggcggggcggctgtggggaggaaggtgacgtgcaggggaccagaggctctgcactgctcctaggacagctcatctgtaatcagaaaaaaaataaacaaaatacagaacgctgactcctccgtgagacagatcggggaccttagcactttaatccctcccttctgagcgctcggtgtgcacttttagactatagctgtttcattgacgtgtcactctccatccagtgtccttgatgtggcttttagagacttagcagaaaattcgacacaagcaggaacttgattttttaagaaaaaatattacattttgaggacattttgacaagtaggggaagagagggcttctgttgttttgttttgttttgttttgttaactaaacctgaagtattaattccacaaagacactgtccctcaggaccactcaggtacagctctgccagggacagagtcctgctagtgggaggtctcaggtggggcggtgtgttctgtgccatgaggcagcgacaggtccagatggatgtcgtcaccaccttcctcagctctcatcacctggtcgtacgccaggcccacctcttcccagcaagggacgccaaagaactgcagtttttattctgagtcttaatttaacttttcatcatcttttcctattttggagaattttttgtaattaaaagcaattattttaaaatgtgcaagccagtatctcacaaggcatggatttctgtggaatttatttttattcaaataaccatatttatctccaggctgtggaatcgccactttctttgtgaagacagtgtctctccttgtaatctcacacaggtacactgaggaggggacggctccgtcttcacattgtgcacagatctgaggatgggattagcgaagctgtggagactgcacatccggacctgcccatgtctcaaaacaaacacatgtacagtggctctttttccttctcaaacactttaccccagaagcaggtggtctgccccaggcataaagaaggaaaattggccatctttcccacctctaaattctgtaaaattatagacttgctcaaaagattcctttttatcatccccacgctgtgtaagtggaaagggcattgtgttccgtgtgtgtccagtttacagcgtctctgccccctagcgtgttttgtgacaatctccctgggtgaggagtgggtgcacccagccccgaggccagtggttgctcggggccttccgtgtgagttctagtgttcacttgatgccggggaatagaattagagaaaactctgacctgccgggttccagggactggtggaggtggatggcaggtccgactcgaccatgacttagttgtaagggtgtgtcggctttttcagtctcatgtgaaaatcctcctgtctctggcagcactgtctgcactttcttgtttactgtttgaagggacgagtaccaagccacaagaacacttcttttggccacagcataagctgatggtatgtaaggaaccgatgggccattaaacatgaactgaacggttaaaagcacagtctatggaacgctaatggagtcagcccctaaagctgtttgctttttcaggctttggattacatgcttttaatttgattttagaatctggacactttctatgaatgtaattcggctgagaaacatgttgctgagatgcaatcctcagtgttctctgtatgtaaatctgtgtatacaccacacgttacaactgcatgagcttcctctcgcacaagaccagctggaactgagcatgagacgctgtcaaatacagacaaaggatttgagatgttctcaataaaaagaaaatgtttcactactcaaaaaaaaaaaaaaaaa

SEQ ID NO: 10 - protein, 701 aa, Homo sapiens
MDIEDEENMSSSSTDVKENRNLDNVSPKDGSTPGPGEGSQLSNGGGGGPGRKRPLEEGSNGHSKYRLKKRRKTPGPVLPKNALMQLNEIKPGLQYTLLSQTGPVHAPLFVMSVEVNGQVFEGSGPTKKKAKLHAAEKALRSFVQFPNASEAHLAMGRTLSVNTDFTSDQADFPDTLFNGFETPDKAEPPFYVGSNGDDSFSSSGDLSLSASPVPASLAQPPLPVLPPFPPPSGKNPVMILNELRPGLKYDFLSESGESHAKSFVMSVVVDGQFFEGSGRNKKLAKARAAQSALAAIFNLHLDQTPSRQPIPSEGLQLHLPQVLADAVSRLVLGKFGDLTDNFSSPHARRKVLAGVVMTTGTDVKDAKVISVSTGTKCINGEYMSDRGLALNDCHAEIISRRSLLRFLYTQLELYLNNKDDQKRSIFQKSERGGFRLKENVQFHLYISTSPCGDARIFSPHEPILEEPADRHPNRKARGQLRTKIESGEGTIPVRSNASIQTWDGVLQGERLLTMSCSDKIARWNVVGIQGSLLSIFVEPIYFSSIILGSLYHGDHLSRAMYQRISNIEDLPPLYTLNKPLLSGISNAEARQPGKAPNFSVNWTVGDSAIEVINATTGKDELGRASRLCKHALYCRWMRVHGKVPSHLLRSKITKPNVYHESKLAAKEYQAAKARLFTAFIKAGLGAWVEKPTEQDQFSLTP

SEQ ID NO: 11 - DNA, 8426 bp, Homo sapiens
  source 1..8426: mol_type = other DNA
tgcgctgggagcccagagcaggaggcagagcccgcgggaagctcggagcactcaggacgccgcgcgcccttccccgccctccctgaccagggagcagctcgctccaggcgcccagccgaggcccccagcccagtgggagcaagtcatagagaacaattcgagagacagagagacggagcgcgctttcctgctcagtcctgaaaagtgagccgctcccgggtttgcaacctcaagcttcgcagcagcggcggcggcggctgccgggaaggaggcaggtgcaggtgcaggagggaggcggctctgggctccgcgcctgggtctcggccatggcctcggtcctggggagcggcagagggtctggagggctgagcagtcaactcaaatgcaagtccaagaggaggaggaggcggaggtccaagcggaaagataaagtaagcatattgtcaaccttcctcgctcctttcaagcacctgagtcctggcatcacaaacacggaggatgacgacaccctcagtaccagcagcgcggaggtgaaggagaaccgcaacgtgggcaacctggccgcgcggccaccgccctccggggaccgggcccggggcggcgcgcccggcgcgaagaggaagcggccgctggaggaggggaatgggggccacttgtgcaaactgcagctggtctggaagaagctgtcgtggtcggtggcgcccaagaacgcgctggtgcagctgcacgagctgaggccgggcctgcagtaccggacagtgtcgcagacgggcccggtgcatgccccggtcttcgcggtagcggtggaggtgaacgggctcacgttcgagggcacaggccccaccaagaagaaggccaagatgcgcgcggcggagctggcactcaggtccttcgtgcagttccccaacgcctgccaggcgcacctggccatgggcgggggcccgggccccggcacggacttcacctccgaccaggccgatttccccgacacgctcttccaggagttcgagcccccggcgccgcgccccggactcgcgggaggccgccccggggacgccgcgcttctgtccgcggcctacgggcgacggcggctgctgtgccgcgcgctggacctggtgggcccgacccccgccacccccgcggccccgggcgagcgcaaccccgtggtgctgctgaaccgcctgcgcgccgggctgcgctacgtgtgtctggcagaaccggccgagcggcgcgcgcggagcttcgtgatggccgtgagcgtggacggcaggacgttcgagggctcggggcgcagcaagaagctggcccggggtcaggccgcgcaggccgcactgcaggagctgttcgacatccagatgcccggccacgcgcccggcagggccaggaggacgccaatgccgcaggaattcgcagactccatatcccagctggtcacacagaagttccgcgaggtgacgacggacctcacgcccatgcacgcccgccataaagcgctggcaggaatcgtcatgaccaaaggcctggatgctcggcaggcgcaggtcgtggccctgtcctcggggaccaagtgcatcagcggcgagcacctcagtgaccaggggctggtggtgaatgactgccacgcggaggtcgtggcccggcgggcgttcctgcacttcctctacacgcagctggagctgcacctgagcaagcggcgcgaggactcagagcgatcgatattcgtgcggttaaaagaaggtggctaccggctgcgagagaacatcctcttccatctctacgtgagcacctccccctgtggagacgcaagactccactctccctacgagatcaccacagacctgcacagcagcaaacacctcgtcaggaagttccgcgggcacctgcgcaccaagatcgagtccggggaagggacggtccccgtgcgtggccccagcgcagtgcagacctgggacggcgtcctgctgggggagcagctgatcaccatgtcctgcacggacaagatcgccaggtggaacgtcctggggctgcagggcgcgctcctgtcccacttcgtggagcccgtgtacctgcagagcatcgtggtgggcagcctgcaccacacgggccacctcgcacgcgtcatgagccaccgcatggagggtgtcggccagctgcccgcctcctaccggcacaaccggcctctcctcagcggcgtgagtgacgccgaggcgcgccagccggggaagtcgccccccttcagcatgaactgggtcgtgggcagcgcggacctggagattatcaacgccaccactgggcggaggagctgtgggggcccatcccggctctgcaagcacgtgctgtctgcacggtgggcgcggctgtatggcaggctgagcacacggacacccagccctggagacacgccctccatgtactgtgaggccaagctgggggcgcacacctaccagtctgtgaaacagcagctgttcaaggcctttcagaaggctggcctgggcacctgggtgaggaaaccaccggagcagcagcagtttctactgactctctaggctgcgggctcctggctgctggagctgagcgggacgctggagggatgggaccgtgtctggggggcgacgtggcgggtcggccggttccctgcattcgttttactttggtgtcccagaaacacgcgagtgtgcaatgtttggacgagcaacaacacaaattcagaacgtgcctctttccagatcgctggccccagaaccctgtcccccacacccaggggcacatgcactgttgagttagcgccgactcttcctgtggagtctgagggaggggctccattcaggcaaaggggttttagctgcagccttggaaggaggcaccgacacgacaccaggcaggagtgagcctcaggccccgtccctgcaccccacccctgcgtgcgcctcttggtgatgctggggtctcactagcttgagggggcacatgaagataagccacaaatgaagagaaaagccatgcccaccccagccccagagaaaccaataagaatcctctattattttcactattcatttaggtttttatactccacctcctttcaaaaaagatttaagatgtacgacattaccgaacacctaaaatagaaccagagaaacgaaagccattcccacaaagtgaaggaacagtttccaaaacccctgcgaggcagagttaagacctgtgacgtggggagcctggaaacacccggcccacgcgtgctgcagatgtggggagcctggaaacacccggcccacgcgtgctgcagatgtggggagcctggaaacacccggcccacgcgtgctgcagatgtggggagtctggaaacacccggcccacgcgtgctgcagatgtggggagcccggaaacacctggcccatgcctgttcagtccgtcatggcgtttggtttatggtggccgatactaagagccactgaggtctgggttgtgtaagggttagctaccaccctataacctccctaccaggtaataaggagaccaagtctttgatcaaattctgattttttaactaaattttcattagaaagcaagcacaaaataaaatcaaaacaacctttaccttcagcttgagaggctaccagctctctattctttcagattctccaatcaacaccttgcaggacaagaccccaaaaaagttcagctgtcaaaaaatgtaagaggcaggattctgtgaccctcagagcaggcttccaacaccagagaccttcagctcacagtggagatgggacatgggggctttagcccacagcatcatccctgagagccaaggcgctgtattcatacggggtcaggctcttttccaagtatctttacgtaatccatgtacctcgagctgctgtcatctcacctccgagaactcatttctaacattcatcatgctagatagccagtctctcatttctccaacttaaaaaatccatctctgaaattcatgacagtgatggcagacggcaaagcatggtggtgtccacctctggggtgtgggcttcccggaaagaatcccagctgaccaccccagaaagtgcaagaataacattgtgctgtacatagtcaagtgctataaattacagttatcataatgagaaggcagaaatgaaactgagttgtagctatttgttacaagataataagaagtagagatattagtatggaaaaagaaacttttcctgggatttaaaatcaggccaaatttatcactcgagttcttttagaaaagaaaacaggtactggagtagacaacttcttccagataaagtttcaaaagatacgagaacattttttcaaataaaggactcacattaatcttttataaaaatcacaaaaataaatgaaatcttaattcagtgcaaaagctaagctcaaacgaccagtttctattaagttctggtaacttgacatcactaggatgaagcttagagaacttagagtcgagaggtgatgggtggctcagacattcgcagcagtcagaagtgagcgctcccctgaatgcgtgggagcacggccagctcctggctgaagtctcccacccaggcctcaaatcaccttcatctgagatgttggctagagaagcatataatgctttgtcccagtgggctccaaatcacgctgtttctgcagcgagggggcctgtgttgtaagcagatttcactgtcacagggaagacgcagggtctgattcacgggactcggaaagcgctcggccacccagtggccagcatcatgaagccacagctggggggttgggattcgttttgtctcaggcattgaccttccagaaacaactcagactttactgaaatatcaacatccacatctggagaaagccacacattaattccatgctacccattagtacaagtgtatggattcgtgtattaagtccacaggagactccatggaaggagagattaaagctcttcagagaaggccgatagcgacagaagagtttccaatgcagccagagtgaggttgaggtgccggagcctttccgtggagggatctgtcagaggggatgtgggctgcagaggggaccccacgtaaggaaaggagggtcccaggccctgggtagtgaaaggagaaggtagtcgaggcagtcaagggtgaccctacacgcaggaggcacgccacaccagatcacgccacacgcaggctgctcccaactggcagggcctgacccagccaggaagggccaggtcctaggattccacagtgccaggagtgcccgaaggggccaggtcccaggattcccacagccccaggtgtgcctggaagacccgggtcccaggattcccacagccccagctatgcttgggggggccgagtcccaggattcccacagccccaggtgtgcctggaagacccgggtcccaggattcccacagccccaagagttcccggaagagctgggtcccaggattcccacagccccagctatgcttgggggggccgggtcccaggattcccacagccccaggtgtgcctggaagacccaggtcccaggattcccacagccctggatgtgcctggaggggctgtgccacctctgcagagatggaagggctcgtgttgaagcctgaggatttcgccctcaggtccaggtcagaacatcccagcaccactttcaggccatgtctgccctgcagttttgaccactgaattggtcttaagagtggccacccattcccctccaagtgagggggcaagacacaagtaggggacgggtcactttctgtcaccctgaaaatacgctgggcacgacacaagtaggggatgggtcactttctgtcaccctgaaaatacgctgggcacgacacaaggggggctgatgaagtcaggcccgcccctccaagtccacctcgaaatcaccgtctggcttcctcgcacttcccaccacctgacaccgccaagccccttgcttcagattttctgtggtctgtattcatacccttgcttcagattttctgtagtctatattcatgcctgcttaggcccctacaaaagtcctcataaaaccagcaccacctttatctgcctccaaaacacacactgctttcccatcaggaagatcagcaatgtggtttccggcctggttcttagtcaaaaagttagtgtctggtggtttctggcctttttgcaggacggggcgcagggaacggctgcgcggccaggcagcctccgctgtccacctccagccccggttttgcagccgtgtgtgatatcaagggatagagttagggtttctgtgggttttgtttttgctgaggaaaggatgtgcaggtcgatagctattgtttcctttccattcctgtgattctctttacatctgtatatatctatcacaccatgctgactttgtgatgagtcctgttcctcagaaacatcaagctgggttccttttcaaagaaacacacagatcatatttctccatctcattttgttttcacagggagcttttcttatcaacaaagtcgcatgcttttttttttggatataatgtttatcacgtctggagagccataaacagtaaatacagaggatatcaatgcccagaatagttattttgatgaaatcctatttttagataataaatttcaagatgtgtctatagtgtattgtttaaaacaactgaagacatttgagacgtacataaagtagacatttttaatttagggaaacttatatgcccttttttaagaagccattctaataaaataatctgactaattggcccaaaatacaataagtatcactttctaacagagaccaaagggaagctgagaggctttccttttatgtactaccgttggatcgctgcagccgcctctcataacacccaacagaacccggtgaggctctgtgggcttcccgcggccctcagggaggctgcagagtcccctagtccatgtcagggagccgccatcccacatacctccaaagcctgtcctcgcctgcagtttttgcagagctcgcggttggaggtggaaatttagaagccctgtgttgcaggagaggcagtagcacccccaggcagctcttagcagggacagaccaccccccgcctcgctggtattttagggtcttttggtattttgccactgtgtggggctaggcgggtggctggaggacacggtgtaggccttgccgctgtctgggttcctcgccactgcaggagcagggctgtttctggaaacactgggctgctggtgacccgtcaaacttctcccacaaattctgaatccgagaaagtgaaggaaggatggtggggaagtgaggaggcaggagcagaggccacagggaccaaccagagatgcggtggagacagaggagcttccttctcaggctgtttctgggaagcctgagaggcggccagcaccaccctccgctcactctcccctcagcctcttcgcttcccttctcaaccctttctctcctcctcgcctcttccctttcttgctgtcttgttagtccctgtcccaaaactcctggctcctttgttctgctgccgtggcccccacccaggaggaggtctcaggcaccatccccccacgagggatccacacaacaggttcatgctggggctgggggagccccgctgggttcctgatgccttgtgcacagggagttgctgcagtcatttttggactctcctgaatgtgtccacatgttctgacctcccaccagaaggaacgctggtggccacatctctagagatctatttacttttttgagaccggcttatgagattggctaatttttgtatttttggtagagatggggtctcaccatgttgcccaggctagtctcgaactcctggcctcaagtgatctgcccacctcggcctcccagagtgctgggattacaggcatgagccaccgcgctggcctctagagattcacaccaaacaatatcactcctgcgggaaccgcagacagatcaaaccctggtgagaggaacactttatttcccaactcatcatcctaagccaaggttggagggatgagcattccctaaacaccaaggcgagatcacctggtcccagtgcctcttttcacacaggccctagatttctatctctctgcagtttatgcatcggtaaaaaaaaaaaaatctctcccagtgtgccccgttagttttcagcattttgtaagcaaaatgaacttaacacatagtaattctaattgaaggtatgtacataaaaagcatgatagaatggcaatattgtatcaatggatgtacatttgtaatatttgtaaaaaaaaatccaaaaccttaaaatatgaatttacatgttaatttgcctctaagttctataaattgcacttcagtgatatctaataagtgaatgtttctgttaagtaaataaaaatattcagtaaaattgtt

SEQ ID NO: 12 - protein, 739 aa, Homo sapiens
MASVLGSGRGSGGLSSQLKCKSKRRRRRRSKRKDKVSILSTFLAPFKHLSPGITNTEDDDTLSTSSAEVKENRNVGNLAARPPPSGDRARGGAPGAKRKRPLEEGNGGHLCKLQLVWKKLSWSVAPKNALVQLHELRPGLQYRTVSQTGPVHAPVFAVAVEVNGLTFEGTGPTKKKAKMRAAELALRSFVQFPNACQAHLAMGGGPGPGTDFTSDQADFPDTLFQEFEPPAPRPGLAGGRPGDAALLSAAYGRRRLLCRALDLVGPTPATPAAPGERNPVVLLNRLRAGLRYVCLAEPAERRARSFVMAVSVDGRTFEGSGRSKKLARGQAAQAALQELFDIQMPGHAPGRARRTPMPQEFADSISQLVTQKFREVTTDLTPMHARHKALAGIVMTKGLDARQAQVVALSSGTKCISGEHLSDQGLVVNDCHAEVVARRAFLHFLYTQLELHLSKRREDSERSIFVRLKEGGYRLRENILFHLYVSTSPCGDARLHSPYEITTDLHSSKHLVRKFRGHLRTKIESGEGTVPVRGPSAVQTWDGVLLGEQLITMSCTDKIARWNVLGLQGALLSHFVEPVYLQSIVVGSLHHTGHLARVMSHRMEGVGQLPASYRHNRPLLSGVSDAEARQPGKSPPFSMNWVVGSADLEIINATTGRRSCGGPSRLCKHVLSARWARLYGRLSTRTPSPGDTPSMYCEAKLGAHTYQSVKQQLFKAFQKAGLGTWVRKPPEQQQFLLTL

SEQ ID NO: 13 - DNA, 5015 bp, Homo sapiens
  source 1..5015: mol_type = other DNA
gagccgaaaggaaccggcgctttgtgacgtcacgtccgccttaccggcggattaagctcgctttcttctaggcgcgtttcctgaaggtcgatggccaggtggtcttcataaactatactgccttgtgatgcctccctagaaatgagaggtctcaataccagctcagaccatgtggaccgcggatgagattgctcagctatgctatgaacactatgggatcaggctgcccaagaaggggaagcctgagccaaaccatgagtggacattattggcagcggtggtgaagatacaatctccagctgacaaggcctgcgacacccctgataagccggtgcaagtgacaaaggaagttgtgtcaatgggaacaggaacaaaatgcataggacagtccaaaatgaggaagaacggagacatcctcaatgatagccatgctgaggtcatagccagaaggagtttccaaaggtaccttctccaccaactccagttggcagccaccctgaaagaggatagcatctttgtcccaggaactcaaaaaggagtgtggaaacttagacgagacctcatttttgtgtttttctccagccatacaccctgtggggatgcctccatcattccgatgcttgagtttgaagatcagccttgctgtcctgtcttcagaaattgggcccacaactcatcagtagaagccagtagtaacctggaagctcctggaaatgaaagaaaatgtgaagaccctgacagtcctgtaaccaaaaagatgaggcttgagcctgggactgcagccagggaggtcaccaacggagcagctcaccatcagagttttggcaagcagaaaagtggcccaatctcaccaggcatccacagctgtgatctcactgtagagggactggctactgtcaccagaatagcccctggtagtgccaaagtgatagacgtttatagaactggagccaagtgtgtacctggagaagctggagactccggaaagccgggtgctgcgtttcaccaggtggggctgctccgagtgaagccaggccgtggagacagaacacgctccatgtcctgtagtgacaagatggcccgatggaacgtcctcggatgccaaggggcactgttgatgcacttgctggaagagcccatctacctgtcagctgtggtcattgggaagtgcccatacagccaggaagccatgcagagagcactgattggaaggtgtcagaatgtgtctgctttaccaaaaggcttcggagttcaagaattaaaaatactgcagtcagatttactatttgaacagagccgcagtgcggtgcaggcaaaaagggctgatagcccaggtcgacttgttccttgtggggcagccatcagctggagtgcagttcctgagcagcctttggatgttactgccaatggctttccacagggaacaacaaagaaaacaattggaagccttcaggcaagatcccaaatcagcaaagtggaactcttcagatcattccagaagctgctaagcagaattgcaagggacaagtggccacactccctcagggtgcagaagctggatacctaccaggagtacaaggaggctgcgtcctcttaccaggaagcctggagcacactccggaagcaggtgtttggatcctggatcagaaacccaccggattatcaccagttcaagtgagaaggaaatgtttgctctggtagcaggacccttcatcctgaactgccttcctccacgcaggacaagtcttagcttttttggtagttaaatcagggaacattctttctgagctttgcatctctgttgtgtaatacagatggaatctggaacttagagcaagctatacttgattgaagtagctctctgctaaaatggcatctcactaatgacctcactatctgacttccattgtgttttattgcttctctaaaatatgaaatcagagtttggaacatgatgctttgaaagtactctgcagcagctctgttttatgggttcctacattgattcagcaaatatttattgagctgaaatgatgtttgacgctctgcaagttttacagctttgcctagcatagcgaatatatattttgtattcccagcctttctttcttcagggagccattcttttcccattcttcagtgatcctgctctgtgcctcagacttggctgaagctaaccttactccctggctctaggaatagcctgtgagccagccctggactgccctggacttgttctcagaggtatcaggaaaggctttctgttttgatctggagctgtaagggtcacataatcatgggactgccggtggccatcttttcctactacctagagaaagccagcaaagaatgaaggcaacaaaggaaacagccaagagaatcaaagatctagtaacactgtttagatgtctcaattatgtgagacagattcctgttgttattttgattaagcctgtttgtgttggttttctgacttacaattgaaagagttctgtccaaaaagccaaaaggtggccaggcttggtggctcaggcctgtaatcccagcactttgggaagcccaggcaggcagatcacttgaggtcaggagttcaagaccagcctggccaacctggtgaaaccctgtctctactaaaaatgcaaaaactagccgggcatggtggcgcacacctgtaatcctagctactcgggaggctgaggcaggagaattgcttgaacccaggaggcggaggtagtggtgagcaaagatcgtaccgctgcactccagcctgggtgatagagcaagaccctatctcaaaaaaaaaaaaaaaaaaaaaaaaagaagccaaaaggccctgcagcaaatcagcaatagacccagcagccaatcctgttttaacatcgaatgtttaacactcaacttcagaccttgaatgattgactgaaaatggaagctcccatgttggggttactcaaggtccttattggttcttcagcattttgtggctataaatatttttaaataacagttgtattgagatataattcacagaccataaaattcacccaactaaagtatacattttaaaagtttttaggccgggcacggtggcttacgcctgtaattccaacactttgggaggctgaggcaggcagatcacctgaggtcaggagttcgagaccagtctggccaacatgatgaaaccccgtctctgctaaaaatacaaaaattagccgggcatggtggtgcacgcctgtagtcccagctacttgggaggctgaggcaggagaatcacttgaacctgggaggcggaggttgcagtgagtcaagatcatgccattgcactccagcctgggtgacagagtgagatcctgtctcaaaaaaaaaaaaaaaaaatttttttagtatatccacagagttgtgcaaccatcaattttagaacattttcatcacaaattttgtgcctgtaatagtttcctagagctgtttcttaacgaagtaccacaagctgggtggcttaagacaacagaaatgtattcctggccgggtgcagtggctcacgcctgtaatcccaacactttaggaggccgaggtgggcagatcacgacgtcaggagatcaagaccatcctggccaacatggtgaagcccctttctactaaaaatacaaaaattagccaggtgtattggcgcacgcctatagtcccaactactcaggaggctgaggcagaagaatctctcaaacccaggaggtggaggttgcagtgagccaggatcgtgccactgcactctagcctgggtgacagagcaagactccatctcaaaaaataagaagaaaagactgtatcccctttcagttctggaggctagaagtccaaacaggttgttggtggggcttctgcttcctctaaaagctttagggcaggggcccccagccccagagccacagaccattacttgtctgtggcctgtcaggaacaaggtcacacagcaggagataagtaacaggtgagcacttgaagtttcatctgtatttatggctgccccccatcgctcacatttctgccgtagctcctcctcctgtcataccagcagtggcattagattctcataggagcgtgaatcctattgtgaactgtgcatgcaagggatctaggttgcacactccttatgagaatataatgcctgatgatctgtcagtgtcttccataacccccatatgggactgtctagttgcaggaaaacaagctcagggctcctactgattctaccttatggtgagttgtataattatttcatcatgtattacgatgtaataataatagaaatacagtgcacagtaaatgtaattcacttcagtcatcccgaaactatccccctatccccaccccggatctgtggaaaaattatcttccatgaaaccagtccctggtgccaaaaaggttggggactgctgctctaaggaagactttttcgtgccttggggactgctgctctgaagaagactctttcatgcttcctccttagcttctgatggctcctggcagtccttggcttgtggctgcatcacttcagtttctccctccgtcttcacatggccttctgtgtctgtgtctcgcatggccttcttccctgtgtctgtgtctttccatggcctacttataaggaccccagtaattggatttagggcctgccctaatccagtctgatttcatcttaactaactagatctgcaaagactctatttccaactaaagtcacattctgaggtttcaggtagacatgaagttttgggggacaccactcaacccattgcagaacccattagcagtcacttcccgttcctcaacgccccctaccccagggcaatcagtaatctactttctgtctgtttggatttgtctattctgaacatttcatataaatggaatcatagaatatgtggtcttttgtgactggcttctttcacttaacatgttttcaagatttatgttgtagcatgaatcagtactttattactatttatggaataaaaatc

SEQ ID NO: 14 - protein, 502 aa, Homo sapiens
MWTADEIAQLCYEHYGIRLPKKGKPEPNHEWTLLAAVVKIQSPADKACDTPDKPVQVTKEVVSMGTGTKCIGQSKMRKNGDILNDSHAEVIARRSFQRYLLHQLQLAATLKEDSIFVPGTQKGVWKLRRDLIFVFFSSHTPCGDASIIPMLEFEDQPCCPVFRNWAHNSSVEASSNLEAPGNERKCEDPDSPVTKKMRLEPGTAAREVTNGAAHHQSFGKQKSGPISPGIHSCDLTVEGLATVTRIAPGSAKVIDVYRTGAKCVPGEAGDSGKPGAAFHQVGLLRVKPGRGDRTRSMSCSDKMARWNVLGCQGALLMHLLEEPIYLSAVVIGKCPYSQEAMQRALIGRCQNVSALPKGFGVQELKILQSDLLFEQSRSAVQAKRADSPGRLVPCGAAISWSAVPEQPLDVTANGFPQGTTKKTIGSLQARSQISKVELFRSFQKLLSRIARDKWPHSLRVQKLDTYQEYKEAASSYQEAWSTLRKQVFGSWIRNPPDYHQFK

SEQ ID NO: 15 - DNA, 6270 bp, Homo sapiens
  source 1..6270: mol_type = other DNA
gcgctcctcctctgccgcgggctctgtagctgagtggtggctgggtatggaggcgaaggcggcacccaagccagctgcaagcggcgcgtgctcggtgtcggcagaggagaccgaaaagtggatggaggaggcgatgcacatggccaaagaagccctcgaaaatactgaagttcctgttggctgtcttatggtctacaacaatgaagttgtagggaaggggagaaatgaagttaaccaaaccaaaaatgctactcgacatgcagaaatggtggccatcgatcaggtcctcgattggtgtcgtcaaagtggcaagagtccctctgaagtatttgaacacactgtgttgtatgtcactgtggagccgtgcattatgtgtgcagctgctctccgcctgatgaaaatcccgctggttgtatatggctgtcagaatgaacgatttggtggttgtggctctgttctaaatattgcctctgctgacctaccaaacactgggagaccatttcagtgtatccctggatatcgggctgaggaagcagtggaaatgttaaagaccttctacaaacaagaaaatccaaatgcaccaaaatcgaaagttcggaaaaaggaatgtcagaaatcttgaacatgttctgatgaaagaaccaagtgacccaaagtgacctggacaagattcatagactgaaagctgttgacatcgttgaatcatatgtttatatattgtttttaatctgcaggaaaatggtgtctctcatcatttgctctgttaagggaacaaattagcactttttagaagtctgacaattgtaaacagttattagcttttccagaagctgattcccattttaagatgggggaaaattaaggtttgaggttttagaaattagcaagtagtgcatacccttctagccacaagtgcccagtccaggcaagtgctgacttcttagagaatgtgtggccagacccagggacctggagtgtgtttggactgcagtttgccaccctgagaacaccttctccaggactggcatttcagaatcagattcttcattttttgcagctacgatgttcttccagggcactgggggctgtgacttctctctaaattgtatataagttgtgtatatagagaccataattatatggtccttagaaaagactttgcttttataaagcatttagaaaaaatgcatacttttaaaacaagtgcttgagttgtcacttaaaaattatagcatattgctataataaaaccttatttatgtcttatttgaagatgaatagtcttaaaagataaagacataaatgggacaattgttattgagcaaaaaaccaaattatcccaccctcatggagcttatattctagcaaggggagatggatatgatagattacacagtttattggaggacaataagagttatggcaaaaagcaaaaggaacacagggtaaaggggataggtgccatttggtggtgagaatgctgactgaaaaatagaatgatcaatttaatctgaaacaaatggttatttcttttataatccatataataaatttaaaatctaaaatgtaaaattttgaacacaacactggaaagggtatccacagcaggaagtccccagttcacctccatgactacagggcagctttgcacagccctctgggcgcactgtgtgcctctgcccagaagggggcctcgccgttccaccagaagctcagctccaggccctggaggggctgctgctcctcagttgcatttcttcagtagattcatttccttgatgcaaagcatctgtatttgttggttctgtcatttgagcgatgtctctgacttgtttgttttgaattacattacaggctggaatgtaattgtggtgaaagtatttttatattgctgagagtagcagctaatcacagttacatgcttcagaggacttataattgcttggttttgtgtgtgtgtgtgtgtgtgtgtgtgtgtgtgtgtgtgtttaactgcatttgaaaagttttatggagaatatgcatgattttaaatctgtgataatgttacatgcaccttcaatttcatccactttaaaaattatcttctcattgaattttagtgcttctactagtttgttcctttttgcagttggtcgtaattcatttctggcttcttatgctttcctgcaagcagatttcattgcatttattgtgttcatatcattttcttggggattatttgtaggacaaccaacctggagttttgcctctctagagtaccacccagtaagtctggctgagcatcttatgtccagtaggttcttggtaaacatttgctaaatgaaattactgattgaaatttggggaaaagtgaataagaagactatctaggacaaaaagccaaagccgaaaatagtatatgagcattctagcccagagactgtcgctactaaaagaatgaaggaaataataaagtgatagacagggaaggatagaaaagacttaacaatatacatatgttccgtctttgctgttttggagaatgatggataagtagtgtttcctgattctgaagcatagctgaacaatttaattgtggtttaccatctttttggttccctcttcagtaattaacctatcgaaaatctgtcctaaatgtttggactggggcacagttccctccatcgctttgggagaaaatcattaatatggcatactgcagattggagggcaggaccactgagggtgtcatagacattagctctatggaattctgctagcaatttccaagtgacagtgaggaattatggatatatgttgaggtcattcagcttcctgagtaccacattccccagctacttagacacgggttaaaatattaagatgtcctagttcaacagcttgaattccattgattgatactgatagtgcctgtccaagacaccagctgaaagacttgttttgtgtacaaaatagttctgaaagtggtgagatacaaaaaggttttagaatcactgccctgttgagagaaattagggggaaatgattacatttagaagctgctagagttatccagtgtttgctggtctttgcaacaaactgtggagaatgggtggtatgtaatgctttggtaggcttcaatcactgataaaagatcatgttaaaatatctttgtgctttcttgttacttggcacaaccatctcttcctgtgttgtatttggagtatcatggagagaaaatagatggccaagagcttcagtgtaggcaagaactcttaatttttctttaaactttttactgggaaaagtatatatatataaaatacacacacacacacacacacacacacacacacacacacacacacaaacacaacacaccatggccctttaccccgaaatgcttcagtatagttattgacttaagtaaatttaacattgatatacttgaatctatcatttgtattacagttttgtcagctgacccaataatgtcctgtaaagaagttctcccactaccctataatcccaggtccagtctagggtccagcattacatttacttgtcttgaatccagctttttcttttttttttttttttttgagataggtctcactctgtcgtccagtggcatgatcacagctcactgcagcctcaacctggctcaagcaatccttcctcctcagcctcctgagtagctgggaccacagactcatgtcaccacacctaatttttttttttttttttttttgtagagacaaggtctcactatgttgcccaggctggtcttgaactcctaggctgaagcaatcctccttccttggcctcccaaagcactgggattatagacgtgagccactgcaccggtctgcctttagcttcttttagtctagaacattttcactggctttctttgtcttttatgacattgacatttttaaataatacagtcattttgcctcctttctgttttcttcttctttttttaaataatagaatggtccttgttttaaatttatttgatattttcttgtgattagattcaggtgctggttgatgttaagttcctcacaggatatcacatctggaggcacacaaaggccgtcacaccaaggtgatgtcaattttggtcatctggtcaaggtgttgtcctattccttcactatatagttaccttttttctctgttgcaatgaataagcagtctgtgggaagaggagctgttacattttaaacagaaaatgtatttgacactgatggaaaggagaggaggaaaattaatgacataaatttcaaagcaactattaaattatttgattgcattcttcctcttttactgtctgccaaaattgataaaaaaaatttttctaataagaatgttttaaatagtgatatcttaataagcatcaaaattaagcctgagaaataaattctttccttcctaatttcctcctcagcaaaagtaataattatataaatttcattatgcctgataagatagggttttggaaaatagacctaagatgtttctgatactgcagatgacctatggtgatccaatgggataaacactctaggtaggttgtcatttggtcataaaatatgagttatcttgggtttccatagagacatctagacttaaaatgttgtaagcactgctactttcaaaatgtcagtaaaaatagcaaaagccaaagctcttgaaaaaattacttaaatcttttttaaaagtagtatagcgccttgttaaaaatctgtggtgatgccaaagcttgtctttcccagtggtcctacgtgaactggccttatagccccagggaaaccagacaccaggaattggtttctctgccttttggcaaaggaataagactacattgacttcatctatgaagacaactgccaactatttcctttgtaaattgctaattttgtgtagtgaggaaaggagcgatgggcgacgtgatttttatggattagactggtgagttctgctgaaagtttgacatctttaggatcttacattttcttcaagttgagctaatgaaaacaggctcgtgactatttatcacctgatttctaagtggatattgggttgaacaccacatatccatgactattaaggaggcttcatggtgtagtttgacaaaggctctctccttgaccaaacttcagtcaggccctaagtcctctttttaaccaggcctccaccttggcccccattcttgatgggcctatacagcccagctttagcaagaatcctgctaagctagtttagagagaatcccacatccccaatatctatgaaatttctcatcccctacttttgatgtgtaagtccttggcctcccttcaacgagaagcctgttaagttcattttgcaagaactctactcttgatatctcctcttagtaatttcctaatcactgaccccctcactctgcccattagttataaacccccacatgttctggttgtattcagagctgagcctgatctcttcctcttgttgggatagttttaaaacctgcgatagttttaaaacctatcactgtagtcctgaattaagtcttccttaccttaacaagtgtcaaaataaatttttctttaacatgttgaagcatgaacttgagaatctagagcaggagtccacaaagtatggcccatgggccatatccagcccgctgccggtttcggtaccactcatgacttaaaaatgggtcttacaattctgagtgattgaaaaaaaatcaaaagaaggataatatttagtgacccatgaaccttatatggcaatcaaatttcagtgtccataaataaagttacattggatgacagccatgcccatttgtttctgtgttgtctgtggctgctcgtgtgctacaatggcagagttgagcagtggtgacaaaccatgcgactcacaaaggcctaaaatatttagcgtctggcccttcgagaaaatgttagctgcccctggtctagagtaggtaaaaggctgagattggaagctgcttgttcaaattctgtgattggaaccgaatgatgtggctcattgtacagctcatggtgaattgcttcagtaccatggttttgttttttccttttgaaaagttggtctataaatgtaaaggaaaaatctaagataccaaaatatgttttctggcttagaatgttttatttccttgtatacattttaagagagtggcaaggagaaaagataatgtatcattttatttgggtttagaataaataatacattttatttatgatcaaaaaaaaaaaaaaaaaa

SEQ ID NO: 16 - protein, 191 aa, Homo sapiens
MEAKAAPKPAASGACSVSAEETEKWMEEAMHMAKEALENTEVPVGCLMVYNNEVVGKGRNEVNQTKNATRHAEMVAIDQVLDWCRQSGKSPSEVFEHTVLYVTVEPCIMCAAALRLMKIPLVVYGCQNERFGGCGSVLNIASADLPNTGRPFQCIPGYRAEEAVEMLKTFYKQENPNAPKSKVRKKECQKS

SEQ ID NO: 17 - DNA, 1801 bp, Homo sapiens
  source 1..1801: mol_type = other DNA
catgtgagcgcagtgccggagcggggcggggcgaggcggcggcgcgcagtgcctactagcaattgtagtccttgccccaccttggcctggcgaagatcccgcccctggaactacgctcccgtcgtgctccgcggctccaggcggagacgaggcggggtgaggtgggttgggccgggccggttgctaagacttggcgaagcgctgcgctcgcgcccggatccctcaggggttagcaggacatgagggagtgtagctctgatgcggtgacctgggccggagccctcggactagcctcagctttggtggcagcacgccctgccttgtggagccacggcctcccgggacggactccccggctctcccccagccgcccgcagccgccggatgatcctctgctcccgtctctgtctcccacagtcggcctcgctgaggatggagcccgccccgggcctcgtggagcagcccaagtgcttggaggccgggagcccggagcctgagccggcgccgtggcaggccctccctgtcctgtccgagaagcagtcaggggacgtggagctggtgctggcctacgccgcgcccgtcctggacaagcgccagacctcacgcctcctgaaggaggtgtcggccctgcacccgctccccgcccagcctcacctcaagcgggtgcggcccagccgcgatgccggcagcccccacgccctggagatgctgctttgcctggctgggccggcctcgggcccgcgctcgctggctgagctcctgccacggccggctgtggacccccgcggcctggggcaacccttcctggtgcccgtgcccgcccggccgcctctgaccaggggccagttcgaggaggcccgggcccactggcccacgtccttccacgaggacaagcaggtgaccagcgccctggctgggcggctcttctccacgcaggagcgcgccgccatgcagagccacatggagcgggcggtgtgggcggcccggcgggcagcagcgcggggcttgcgggccgtgggggccgtggtagtggacccggcctcggaccgcgtgctggccaccggccacgactgcagctgcgcggacaaccccctcctgcacgccgtcatggtgtgcgtggacctcgtggcgcgcggccagggccgcggcacctacgacttcagacccttccccgcctgctccttcgccccggccgctgccccccaggccgtccgcgcaggcgccgtgcgtaaactggacgcagacgaggacggcctcccctacctgtgcactggctacgacctgtacgtgacccgcgagccctgcgccatgtgcgccatggccctggtgcacgcacgcatcctgcgcgtcttctacggtgcgccctcgcccgacggcgccctgggcacccgcttccgcatccacgcacggcccgacctcaaccaccgcttccaggtgttccgcggggtgctggaggagcagtgccgctggctggaccccgacacgtaggcgccgccctcctgcctccggacccttcccgctcccggccgtggggcgcccctcctggacttccgggcctcgatttcttccgcacaagcctgaccgtggatttcagggacacataccgcctccagcggggagcacgggtgctgccttccgtgcggatcgagctttcctggactcggtcattggggccaccccgtgccagcggtgcccttctgcggccgcccttgctgcgtttgtgtcccctctgtctcgcgggccgggaaactgctctgatgggaaaataaacagcccaaaaccaaaaaaaaaaaaaaaaaaa

SEQ ID NO: 18 - protein, 367 aa, Homo sapiens
MILCSRLCLPQSASLRMEPAPGLVEQPKCLEAGSPEPEPAPWQALPVLSEKQSGDVELVLAYAAPVLDKRQTSRLLKEVSALHPLPAQPHLKRVRPSRDAGSPHALEMLLCLAGPASGPRSLAELLPRPAVDPRGLGQPFLVPVPARPPLTRGQFEEARAHWPTSFHEDKQVTSALAGRLFSTQERAAMQSHMERAVWAARRAAARGLRAVGAVVVDPASDRVLATGHDCSCADNPLLHAVMVCVDLVARGQGRGTYDFRPFPACSFAPAAAPQAVRAGAVRKLDADEDGLPYLCTGYDLYVTREPCAMCAMALVHARILRVFYGAPSPDGALGTRFRIHARPDLNHRFQVFRGVLEEQCRWLDPDT

SEQ ID NO: 19 - DNA, 2815 bp, Homo sapiens
  source 1..2815: mol_type = other DNA
aatgcactgtcagactaagacagagaaccatcattaattgaagtgagatttttctggcctgagacttgcagggaggcaagaagacactctggacaccactatggacagcctcttgatgaaccggaggaagtttctttaccaattcaaaaatgtccgctgggctaagggtcggcgtgagacctacctgtgctacgtagtgaagaggcgtgacagtgctacatccttttcactggactttggttatcttcgcaataagaacggctgccacgtggaattgctcttcctccgctacatctcggactgggacctagaccctggccgctgctaccgcgtcacctggttcacctcctggagcccctgctacgactgtgcccgacatgtggccgactttctgcgagggaaccccaacctcagtctgaggatcttcaccgcgcgcctctacttctgtgaggaccgcaaggctgagcccgaggggctgcggcggctgcaccgcgccggggtgcaaatagccatcatgaccttcaaagattatttttactgctggaatacttttgtagaaaaccacgaaagaactttcaaagcctgggaagggctgcatgaaaattcagttcgtctctccagacagcttcggcgcatccttttgcccctgtatgaggttgatgacttacgagacgcatttcgtactttgggactttgatagcaacttccaggaatgtcacacacgatgaaatatctctgctgaagacagtggataaaaaacagtccttcaagtcttctctgtttttattcttcaactctcactttcttagagtttacagaaaaaatatttatatacgactctttaaaaagatctatgtcttgaaaatagagaaggaacacaggtctggccagggacgtgctgcaattggtgcagttttgaatgcaacattgtcccctactgggaataacagaactgcaggacctgggagcatcctaaagtgtcaacgtttttctatgacttttaggtaggatgagagcagaaggtagatcctaaaaagcatggtgagaggatcaaatgtttttatatcaacatcctttattatttgattcatttgagttaacagtggtgttagtgatagatttttctattcttttcccttgacgtttactttcaagtaacacaaactcttccatcaggccatgatctataggacctcctaatgagagtatctgggtgattgtgaccccaaaccatctctccaaagcattaatatccaatcatgcgctgtatgttttaatcagcagaagcatgtttttatgtttgtacaaaagaagattgttatgggtggggatggaggtatagaccatgcatggtcaccttcaagctactttaataaaggatcttaaaatgggcaggaggactgtgaacaagacaccctaataatgggttgatgtctgaagtagcaaatcttctggaaacgcaaactcttttaaggaagtccctaatttagaaacacccacaaacttcacatatcataattagcaaacaattggaaggaagttgcttgaatgttggggagaggaaaatctattggctctcgtgggtctcttcatctcagaaatgccaatcaggtcaaggtttgctacattttgtatgtgtgtgatgcttctcccaaaggtatattaactatataagagagttgtgacaaaacagaatgataaagctgcgaaccgtggcacacgctcatagttctagctgcttgggaggttgaggagggaggatggcttgaacacaggtgttcaaggccagcctgggcaacataacaagatcctgtctctcaaaaaaaaaaaaaaaaaaaagaaagagagagggccgggcgtggtggctcacgcctgtaatcccagcactttgggaggccgagccgggcggatcacctgtggtcaggagtttgagaccagcctggccaacatggcaaaaccccgtctgtactcaaaatgcaaaaattagccaggcgtggtagcaggcacctgtaatcccagctacttgggaggctgaggcaggagaatcgcttgaacccaggaggtggaggttgcagtaagctgagatcgtgccgttgcactccagcctgggcgacaagagcaagactctgtctcagaaaaaaaaaaaaaaaagagagagagagagaaagagaacaatatttgggagagaaggatggggaagcattgcaaggaaattgtgctttatccaacaaaatgtaaggagccaataagggatccctatttgtctcttttggtgtctatttgtccctaacaactgtctttgacagtgagaaaaatattcagaataaccatatccctgtgccgttattacctagcaacccttgcaatgaagatgagcagatccacaggaaaacttgaatgcacaactgtcttattttaatcttattgtacataagtttgtaaaagagttaaaaattgttacttcatgtattcatttatattttatattattttgcgtctaatgattttttattaacatgatttccttttctgatatattgaaatggagtctcaaagcttcataaatttataactttagaaatgattctaataacaacgtatgtaattgtaacattgcagtaatggtgctacgaagccatttctcttgatttttagtaaacttttatgacagcaaatttgcttctggctcactttcaatcagttaaataaatgataaataattttggaagctgtgaagataaaataccaaataaaataatataaaagtgatttatatgaagttaaaataaaaaatcagtatgatggaataaacttg

SEQ ID NO: 20 - protein, 198 aa, Homo sapiens [sequence omitted]

SEQ ID NO: 21 - DNA, 567 bp, Homo sapiens
  source 1..567: mol_type = other DNA
atggacagcctcttgatgaaccggaggaagtttctttaccaattcaaaaatgtccgctgggctaagggtcggcgtgagacctacctgtgctacgtagtgaagaggcgtgacagtgctacatccttttcactggactttggttatcttcgcaataagaacggctgccacgtggaattgctcttcctccgctacatctcggactgggacctagaccctggccgctgctaccgcgtcacctggttcacctcctggagcccctgctacgactgtgcccgacatgtggccgactttctgcgagggaaccccaacctcagtctgaggatcttcaccgcgcgcctctacttctgtgaggaccgcaaggctgagcccgaggggctgcggcggctgcaccgcgccggggtgcaaatagccatcatgaccttcaaagaaaaccacgaaagaactttcaaagcctgggaagggctgcatgaaaattcagttcgtctctccagacagcttcggcgcatccttttgcccctgtatgaggttgatgacttacgagacgcatttcgtactttgggactttga

SEQ ID NO: 22 - protein, 188 aa, Homo sapiens
MDSLLMNRRKFLYQFKNVRWAKGRRETYLCYVVKRRDSATSFSLDFGYLRNKNGCHVELLFLRYISDWDLDPGRCYRVTWFTSWSPCYDCARHVADFLRGNPNLSLRIFTARLYFCEDRKAEPEGLRRLHRAGVQIAIMTFKENHERTFKAWEGLHENSVRLSRQLRRILLPLYEVDDLRDAFRTLGL

SEQ ID NO: 23 - DNA, 894 bp, Homo sapiens
  source 1..894: mol_type = other DNA
atgactccagaggaggaagtccagagacagagcaccatgacttctgagaaaggtccttcaaccggtgaccccactctgaggagaagaatcgaaccctgggagtttgacgtcttctatgaccccagagaacttcgtaaagaggcctgtctgctctacgaaatcaagtggggcatgagccggaagatctggcgaagctcaggcaaaaacaccaccaatcacgtggaagttaattttataaaaaaatttacgtcagaaagagattttcacccatccatgagctgctccatcacctggttcttgtcctggagtccctgctgggaatgctcccaggctattagagagtttctgagtcggcaccctggtgtgactctagtgatctacgtagctcggcttttttggcacatggatcaacaaaatcggcaaggtctcagggaccttgttaacagtggagtaactattcagattatgagagcatcagagtattatcactgctggaggaattttgtcaactacccacctggggatgaagctcactggccacaatacccacctctgtggatgatgttgtacgcactggagctgcactgcataattctaagtcttccaccctgtttaaagatttcaagaagatggcaaaatcatcttacatttttcagacttcatcttcaaaactgccattaccaaacgattccgccacacatccttttagctacagggctgatacatccttctgtggcttggagatgaataggatgattccgtgtgtgtactgattcaagaacaagcaatgatgacccactaaagagtgaatgccatttagaatctagaaatgttcacaaggtaccccaaaactctgtagcttaaaccaacaataaatatgtattacctctggca

SEQ ID NO: 24 - protein, 236 aa, Homo sapiens
MTSEKGPSTGDPTLRRRIEPWEFDVFYDPRELRKEACLLYEIKWGMSRKIWRSSGKNTTNHVEVNFIKKFTSERDFHPSMSCSITWFLSWSPCWECSQAIREFLSRHPGVTLVIYVARLFWHMDQQNRQGLRDLVNSGVTIQIMRASEYYHCWRNFVNYPPGDEAHWPQYPPLWMMLYALELHCIILSLPPCLKISRRWQNHLTFFRLHLQNCHYQTIPPHILLATGLIHPSVAWR

SEQ ID NO: 25 - DNA, 872 bp, Homo sapiens
  source 1..872: mol_type = other DNA
tccagaggaggaagtccagagacagagcaccatgacttctgagaaaggagaagaatcgaaccctgggagtttgacgtcttctatgaccccagagaacttcgtaaagaggcctgtctgctctacgaaatcaagtggggcatgagccggaagatctggcgaagctcaggcaaaaacaccaccaatcacgtggaagttaattttataaaaaaatttacgtcagaaagagattttcacccatccatgagctgctccatcacctggttcttgtcctggagtccctgctgggaatgctcccaggctattagagagtttctgagtcggcaccctggtgtgactctagtgatctacgtagctcggcttttttggcacatggatcaacaaaatcggcaaggtctcagggaccttgttaacagtggagtaactattcagattatgagagcatcagagtattatcactgctggaggaattttgtcaactacccacctggggatgaagctcactggccacaatacccacctctgtggatgatgttgtacgcactggagctgcactgcataattctaagtcttccaccctgtttaaagatttcaagaagatggcaaaatcatcttacatttttcagacttcatcttcaaaactgccattaccaaacgattccgccacacatccttttagctacagggctgatacatccttctgtggcttggagatgaataggatgattccgtgtgtgtactgattcaagaacaagcaatgatgacccactaaagagtgaatgccatttagaatctagaaatgttcacaaggtaccccaaaactctgtagcttaaaccaacaataaatatgtattacctctggcaaaaaaaaaaaa

SEQ ID NO: 26 - protein, 191 aa, Homo sapiens
MSRKIWRSSGKNTTNHVEVNFIKKFTSERDFHPSMSCSITWFLSWSPCWECSQAIREFLSRHPGVTLVIYVARLFWHMDQQNRQGLRDLVNSGVTIQIMRASEYYHCWRNFVNYPPGDEAHWPQYPPLWMMLYALELHCIILSLPPCLKISRRWQNHLTFFRLHLQNCHYQTIPPHILLATGLIHPSVAWR

SEQ ID NO: 27 - DNA, 1643 bp, Homo sapiens
  source 1..1643: mol_type = other DNA
acgaagcctggcctgctgggtccttttcccgtcatccccagccagatttagctgctgacagctgcttgggactctgccgccagggcctggcccagacctgcctgcctctctcctctccctcagtgactcctgagccacagcccctccatggcccagaaggaagaggctgctgtggccactgaggctgcctcccagaatggggaggatctggagaacctggacgaccctgagaagctgaaagagctgattgagctgccgccctttgagattgtcacaggagaacggctgcctgccaacttctttaaattccagttccggaatgtggagtacagttccgggaggaacaagaccttcctctgctatgtggttgaagcacagggcaaggggggccaagtgcaggcatctcggggatacctagaggatgagcatgcggctgcccatgcagaggaagctttcttcaacaccatcctgccagccttcgacccagccctgcggtacaatgtcacctggtatgtgtcctccagcccctgtgcagcgtgtgctgaccgcattatcaaaacccttagcaagaccaagaacctgcgtctgctcattctggtgggtcgactcttcatgtgggaggagccggagatccaggctgctctgaagaagctgaaggaggctggctgtaaactgcgcatcatgaagccccaggacttcgaatatgtctggcagaattttgtggagcaagaagagggtgaatccaaggcctttcagccctgggaggacattcaggagaacttcctatactacgaggagaagttggcagacatcctgaagtagggcaactgggctttgcctcacgtattcctgctgccaccaagagacagcaatgacatgtacagccatctgggacatgcctgtcttcctaataccatttggagctggacaacatttgacaccaaccaatcatactggacaaggcccttagaggacttgaaatatacttctcatgctgtagtttatttaggctgtgactctctctctaatgctgctctcgggaaggacgaaagtgacctgcaaggagagaaatgcaaccatacatgggctccagtcaactatgggactgaaggtcctaattgctcacccaagggggctgcttaacacaaacagcctcagacccgaggtttagatttctgaaatatgcattttatgttaagttgggtatttttttaaaaaaagaaaaacagcaacattaataaaagaagtggtgtgtttttcccgtggccagatttttaagaaacttgaacatctggaagttgccacagacaagcgagctagtccagacacagggacactccaggctatgaaccaagccatggttaaaccaagacttgcctagacaatgcagaataactctcccatacagacttcttacgacaatcaagtgagggcctttcccttctctgaaggcaagtaatagggcagaaggtggaacaaaaagcatgagaaatctaaggtagccacatcccaaatgccagttaataagcaactagcttctttattagagaggagcgggtgaaaagtagtagtttaatgttcagatatacaatcatttaaaagtattcaatacatggtgtttgatggtaatggcaaaaaaaaaa

SEQ ID NO: 28 - protein, 224 aa, Homo sapiens
MAQKEEAAVATEAASQNGEDLENLDDPEKLKELIELPPFEIVTGERLPANFFKFQFRNVEYSSGRNKTFLCYVVEAQGKGGQVQASRGYLEDEHAAAHAEEAFFNTILPAFDPALRYNVTWYVSSSPCAACADRIIKTLSKTKNLRLLILVGRLFMWEEPEIQAALKKLKEAGCKLRIMKPQDFEYVWQNFVEQEEGESKAFQPWEDIQENFLYYEEKLADILK

SEQ ID NO: 29 - DNA, 1444 bp, Homo sapiens
  source 1..1444: mol_type = other DNA
ggagaaggggtggggcagggtatcgctgactcagcagcttccaggttgctctgatgatatattaaggctcctgaatcctaagagaatgttggtgaagatcttaacaccacgccttgagcaagtcgcaagagcgggaggacacagaccaggaaccgagaagggacaagcacatggaagccagcccagcatccgggcccagacacttgatggatccacacatattcacttccaactttaacaatggcattggaaggcataagacctacctgtgctacgaagtggagcgcctggacaatggcacctcggtcaagatggaccagcacaggggctttctacacaaccaggctaagaatcttctctgtggcttttacggccgccatgcggagctgcgcttcttggacctggttccttctttgcagttggacccggcccagatctacagggtcacttggttcatctcctggagcccctgcttctcctggggctgtgccggggaagtgcgtgcgttccttcaggagaacacacacgtgagactgcgtatcttcgctgcccgcatctatgattacgaccccctatataaggaggcactgcaaatgctgcgggatgctggggcccaagtctccatcatgacctacgatgaatttaagcactgctgggacacctttgtggaccaccagggatgtcccttccagccctgggatggactagatgagcacagccaagccctgagtgggaggctgcgggccattctccagaatcagggaaactgaaggatgggcctcagtctctaaggaaggcagagacctgggttgagcagcagaataaaagatcttcttccaagaaatgcaaacagaccgttcaccaccatctccagctgctcacagacgccagcaaagcagtatgctcccgatcaagtagatttttaaaaaatcagagtgggccgggcgcggtggctcacgcctgtaatcccagcactttggaggccaaggcgggtggatcacgaggtcaggagatcgagaccatcctggctaacacggtgaaaccctgtctctactaaaaatacaaaaaattagccaggcgtggtggcgggcgcctgtagtcccagctactctggaggctgaggcaggagagtagcgtgaacccgggaggcagagcttgcggtgagccgagattgcgctactgcactccagcctgggcgacagtaccagactccatctcaaaaaaaaaaaaaccagactgaattaattttaactgaaaatttctcttatgttccaagtacacaatagtaagattatgctcaatattctcagaataattttcaatgtattaatgaaatgaaatgataatttggcttcatatctagactaacacaaaattaagaatcttccataattgcttttgctcagtaactgtgtcatgaattgcaagagtttccacaaacact

SEQ ID NO: 30 - protein, 199 aa, Homo sapiens
MEASPASGPRHLMDPHIFTSNFNNGIGRHKTYLCYEVERLDNGTSVKMDQHRGFLHNQAKNLLCGFYGRHAELRFLDLVPSLQLDPAQIYRVTWFISWSPCFSWGCAGEVRAFLQENTHVRLRIFAARIYDYDPLYKEALQMLRDAGAQVSIMTYDEFKHCWDTFVDHQGCPFQPWDGLDEHSQALSGRLRAILQNQGN

SEQ ID NO: 31 - DNA, 1390 bp, Homo sapiens
  source 1..1390: mol_type = other DNA
ggagaaggggtggggcagggtatcgctgactcagcagcttccaggttgctctgatgatatattaaggctcctgaatcctaagagaatgttggtgaagatcttaacaccacgccttgagcaagtcgcaagagcgggaggacacagaccaggaaccgagaagggacaagcacatggaagccagcccagcatccgggcccaggcataagacctacctgtgctacgaagtggagcgcctggacaatggcacctcggtcaagatggaccagcacaggggctttctacacaaccaggctaagaatcttctctgtggcttttacggccgccatgcggagctgcgcttcttggacctggttccttctttgcagttggacccggcccagatctacagggtcacttggttcatctcctggagcccctgcttctcctggggctgtgccggggaagtgcgtgcgttccttcaggagaacacacacgtgagactgcgtatcttcgctgcccgcatctatgattacgaccccctatataaggaggcactgcaaatgctgcgggatgctggggcccaagtctccatcatgacctacgatgaatttaagcactgctgggacacctttgtggaccaccagggatgtcccttccagccctgggatggactagatgagcacagccaagccctgagtgggaggctgcgggccattctccagaatcagggaaactgaaggatgggcctcagtctctaaggaaggcagagacctgggttgagcagcagaataaaagatcttcttccaagaaatgcaaacagaccgttcaccaccatctccagctgctcacagacgccagcaaagcagtatgctcccgatcaagtagatttttaaaaaatcagagtgggccgggcgcggtggctcacgcctgtaatcccagcactttggaggccaaggcgggtggatcacgaggtcaggagatcgagaccatcctggctaacacggtgaaaccctgtctctactaaaaatacaaaaaattagccaggcgtggtggcgggcgcctgtagtcccagctactctggaggctgaggcaggagagtagcgtgaacccgggaggcagagcttgcggtgagccgagattgcgctactgcactccagcctgggcgacagtaccagactccatctcaaaaaaaaaaaaaccagactgaattaattttaactgaaaatttctcttatgttccaagtacacaatagtaagattatgctcaatattctcagaataattttcaatgtattaatgaaatgaaatgataatttggcttcatatctagactaacacaaaattaagaatcttccataattgcttttgctcagtaactgtgtcatgaattgcaagagtttccacaaacact

SEQ ID NO: 32 - protein, 181 aa, Homo sapiens
MEASPASGPRHKTYLCYEVERLDNGTSVKMDQHRGFLHNQAKNLLCGFYGRHAELRFLDLVPSLQLDPAQIYRVTWFISWSPCFSWGCAGEVRAFLQENTHVRLRIFAARIYDYDPLYKEALQMLRDAGAQVSIMTYDEFKHCWDTFVDHQGCPFQPWDGLDEHSQALSGRLRAILQNQGN

SEQ ID NO: 33 - DNA, 1560 bp, Homo sapiens
  source 1..1560: mol_type = other DNA
cacagagcttcaaaaaaagagcgggacagggacaagcgtatctaagaggctgaacatgaatccacagatcagaaatccgatggagcggatgtatcgagacacattctacgacaactttgaaaacgaacccatcctctatggtcggagctacacttggctgtgctatgaagtgaaaataaagaggggccgctcaaatctcctttgggacacaggggtctttcgaggccaggtgtatttcaagcctcagtaccacgcagaaatgtgcttcctctcttggttctgtggcaaccagctgcctgcttacaagtgtttccagatcacctggtttgtatcctggaccccctgcccggactgtgtggcgaagctggccgaattcctgtctgagcaccccaatgtcaccctgaccatctctgccgcccgcctctactactactgggaaagagattaccgaagggcgctctgcaggctgagtcaggcaggagcccgcgtgaagatcatggactatgaagaatttgcatactgctgggaaaactttgtgtacaatgaaggtcagcaattcatgccttggtacaaattcgatgaaaattatgcattcctgcaccgcacgctaaaggagattctcagatacctgatggatccagacacattcactttcaactttaataatgaccctttggtccttcgacggcgccagacctacttgtgctatgaggtggagcgcctggacaatggcacctgggtcctgatggaccagcacatgggctttctatgcaacgaggctaagaatcttctctgtggcttttacggccgccatgcggagctgcgcttcttggacctggttccttctttgcagttggacccggcccagatctacagggtcacttggttcatctcctggagcccctgcttctcctggggctgtgccggggaagtgcgtgcgttccttcaggagaacacacacgtgagactgcgcatcttcgctgcccgcatctatgattacgaccccctatataaggaggcgctgcaaatgctgcgggatgctggggcccaagtctccatcatgacctacgatgagtttgagtactgctgggacacctttgtgtaccgccagggatgtcccttccagccctgggatggactagaggagcacagccaagccctgagtgggaggctgcgggccattctccagaatcagggaaactgaaggatgggcctcagtctctaaggaaggcagagacctgggttgagcagcagaataaaagatcttcttccaagaaatgcaaacagaccgttcaccaccatctccagctgctcacagacaccagcaaagcaatgtgctcctgatcaagtagattttttaaaaatcagagtcaattaattttaattgaaaatttctcttatgttccaagtgtacaagagtaagattatgctcaatattcccagaatagttttcaatgtattaatgaagtgattaattggctccatatttagactaataaaacattaagaatcttccataattgtttccacaaacactaaaaaaaaaaaaaaaaaaaaaaa

SEQ ID NO: 34 - protein, 382 aa, Homo sapiens
MNPQIRNPMERMYRDTFYDNFENEPILYGRSYTWLCYEVKIKRGRSNLLWDTGVFRGQVYFKPQYHAEMCFLSWFCGNQLPAYKCFQITWFVSWTPCPDCVAKLAEFLSEHPNVTLTISAARLYYYWERDYRRALCRLSQAGARVKIMDYEEFAYCWENFVYNEGQQFMPWYKFDENYAFLHRTLKEILRYLMDPDTFTFNFNNDPLVLRRRQTYLCYEVERLDNGTWVLMDQHMGFLCNEAKNLLCGFYGRHAELRFLDLVPSLQLDPAQIYRVTWFISWSPCFSWGCAGEVRAFLQENTHVRLRIFAARIYDYDPLYKEALQMLRDAGAQVSIMTYDEFEYCWDTFVYRQGCPFQPWDGLEEHSQALSGRLRAILQNQGN

SEQ ID NO: 35 - DNA, 1485 bp, Homo sapiens
  source 1..1485: mol_type = other DNA
cacagagcttcaaaaaaagagcgggacagggacaagcgtatctaagaggctgaacatgaatccacagatcagaaatccgatggagcggatgtatcgagacacattctacgacaactttgaaaacgaacccatcctctatggtcggagctacacttggctgtgctatgaagtgaaaataaagaggggccgctcaaatctcctttgggacacaggggtctttcgaggccaggtgtatttcaagcctcagtaccacgcagaaatgtgcttcctctcttggttctgtggcaaccagctgcctgcttacaagtgtttccagatcacctggtttgtatcctggaccccctgcccggactgtgtggcgaagctggccgaattcctgtctgagcaccccaatgtcaccctgaccatctctgccgcccgcctctactactactgggaaagagattaccgaagggcgctctgcaggctgagtcaggcaggagcccgcgtgaagatcatggactatgaagaatttgcatactgctgggaaaactttgtgtacaatgaaggtcagcaattcatgccttggtacaaattcgatgaaaattatgcattcctgcaccgcacgctaaaggagattctcagatacctgatggatccagacacattcactttcaactttaataatgaccctttggtccttcgacggcgccagacctacttgtgctatgaggtggagcgcctggacaatggcacctgggtcctgatggaccagcacatgggctttctatgcaacgagttggacccggcccagatctacagggtcacttggttcatctcctggagcccctgcttctcctggggctgtgccggggaagtgcgtgcgttccttcaggagaacacacacgtgagactgcgcatcttcgctgcccgcatctatgattacgaccccctatataaggaggcgctgcaaatgctgcgggatgctggggcccaagtctccatcatgacctacgatgagtttgagtactgctgggacacctttgtgtaccgccagggatgtcccttccagccctgggatggactagaggagcacagccaagccctgagtgggaggctgcgggccattctccagaatcagggaaactgaaggatgggcctcagtctctaaggaaggcagagacctgggttgagcagcagaataaaagatcttcttccaagaaatgcaaacagaccgttcaccaccatctccagctgctcacagacaccagcaaagcaatgtgctcctgatcaagtagattttttaaaaatcagagtcaattaattttaattgaaaatttctcttatgttccaagtgtacaagagtaagattatgctcaatattcccagaatagttttcaatgtattaatgaagtgattaattggctccatatttagactaataaaacattaagaatcttccataattgtttccacaaacactaaaaaaaaaaaaaaaaaaaaaaa

SEQ ID NO: 36 - protein, 357 aa, Homo sapiens
MNPQIRNPMERMYRDTFYDNFENEPILYGRSYTWLCYEVKIKRGRSNLLWDTGVFRGQVYFKPQYHAEMCFLSWFCGNQLPAYKCFQITWFVSWTPCPDCVAKLAEFLSEHPNVTLTISAARLYYYWERDYRRALCRLSQAGARVKIMDYEEFAYCWENFVYNEGQQFMPWYKFDENYAFLHRTLKEILRYLMDPDTFTFNFNNDPLVLRRRQTYLCYEVERLDNGTWVLMDQHMGFLCNELDPAQIYRVTWFISWSPCFSWGCAGEVRAFLQENTHVRLRIFAARIYDYDPLYKEALQMLRDAGAQVSIMTYDEFEYCWDTFVYRQGCPFQPWDGLEEHSQALSGRLRAILQNQGN

SEQ ID NO: 37 - DNA, 1127 bp, Homo sapiens
  source 1..1127: mol_type = other DNA
ttaaagagggctgctcaactgcaaggacgctgtaagcaggaagagaagccacagcgcttcagaaaagagtgggacagggacaagcatatctaagaggctgaacatgaatccacagatcagaaacccgatgaaggcaatgtatccaggcacattctacttccaatttaaaaacctatgggaagccaacgatcggaacgaaacttggctgtgcttcaccgtggaaggtataaagcgccgctcagttgtctcctggaagacgggcgtcttccgaaaccaggtggattctgagacccattgtcatgcagaaaggtgcttcctctcttggttctgcgacgacatactgtctcctaacacaaagtaccaggtcacctggtacacatcttggagcccttgcccagactgtgcaggggaggtggccgagttcctggccaggcacagcaacgtgaatctcaccatcttcaccgcccgcctctactacttccagtatccatgttaccaggaggggctccgcagcctgagtcaggaaggggtcgctgtggagatcatggactatgaagattttaaatattgttgggaaaactttgtgtacaatgataatgagccattcaagccttggaagggattaaaaaccaactttcgacttctgaaaagaaggctacgggagagtctccagtgaggggtctccctgggcctcatggtctgtctcctctagcctcctgctcatgctgcacgggcctcccctccaccctggacccgctctgtttctgcctggtcatcctgagcccctcctggcctcagggccattccacagtgctcccctgcctcaccgcttcctcctcgctcttccagactcttcctgcagaggctcctttctgcctccatggctatccatccacccccacagaccccgttcctccagcctgcgtgcccctaacctggcttttcccatctccccagcataaccaaatcttactaaactcatcctaggctgggcatggtgactcacgcctgtaatcccccagcaatttgggaggcaaaggtgggagaatcgcgtgagcccaggagttccagaccaggctgggtcacatgacaaagccccatctctacaaaaaaaaaaaaaaaaaaaa

SEQ ID NO: 38 - protein, 190 aa, Homo sapiens
MNPQIRNPMKAMYPGTFYFQFKNLWEANDRNETWLCFTVEGIKRRSVVSWKTGVFRNQVDSETHCHAERCFLSWFCDDILSPNTKYQVTWYTSWSPCPDCAGEVAEFLARHSNVNLTIFTARLYYFQYPCYQEGLRSLSQEGVAVEIMDYEDFKYCWENFVYNDNEPFKPWKGLKTNFRLLKRRLRESLQ

SEQ ID NO: 39 - DNA, 2519 bp, Homo sapiens
  source 1..2519: mol_type = other DNA
tttttttttttttgagaggaagcctcgctctctcacccaggctggagtgcagtggcaggaacttggctcactgcaacctccgcttcccaggttcaagtgattctcctgcctcagcctccagagtagctgggattacaggcgcacgtcaccacacctggctaatttttgtatttttagtagagacggggtttctccatgttggtcaggctggtctcgaactcctgacctcgtgatccgcccgcctcggcctcccaaagtgctgggattacaggcgtgagccaccgtgcccggccgggaggtcactttaaggagggctgtccaactgcaaggagccgcaagcaggaagtgaaaccacagcacttcaaaaaaagagggagactgggacaagcgtatctaagaggctgaacatgaatccacagatcagaaatccgatggagcggatgtatcgagacacattctacgacaactttgaaaacgaacccatcctctatggtcggagctacacttggctgtgctatgaagtgaaaataaagaggggccgctcaaatctcctttgggacacaggggtctttcgaggcccggtactacccaaacgtcagtcgaatcacaggcaggaggtgtatttccggtttgagaaccacgcagaaatgtgcttcttatcttggttctgtggcaaccgactgcctgctaacaggcgcttccagatcacctggtttgtatcatggaacccctgcctgccctgtgtggtgaaggtgaccaaattcttggctgagcaccccaatgtcaccctgaccatctctgccgcccgcctctactactaccgggatagagattggcggtgggtgctcctcaggctgcataaggcaggggcccgtgtgaagatcatggactatgaagactttgcatactgctgggaaaactttgtgtgcaatgaaggtcagccattcatgccttggtacaaattcgatgacaattatgcatccctgcaccgcacgctaaaggagattctcagaaacccgatggaggcaatgtacccacacatattctacttccactttaaaaacctactgaaagcctgtggtcggaacgaaagctggctgtgcttcaccatggaagttacaaagcaccactcagctgtcttccggaagaggggcgtcttccgaaaccaggtggatcctgagacccattgtcatgcagaaaggtgcttcctctcttggttctgtgacgacatactgtctcctaacacaaactacgaggtcacctggtacacatcttggagcccttgcccagagtgtgcaggggaggtggccgagttcctggccaggcacagcaacgtgaatctcaccatcttcaccgcccgcctctgctacttctgggatacagattaccaggaggggctctgcagcctgagtcaggaaggggcctccgtgaagatcatgggctacaaagattttgtatcttgttggaaaaactttgtgtacagtgatgatgagccattcaagccttggaagggactacaaaccaactttcgacttctgaaaagaaggctacgggagattctccagtgaggggtctccctgggcctcatggtctgtctcttctagcctcctgctcatgctgcacgggcctcccctccatcctgcaccagctgtgcttttgcctggtcatcctgagcccctcctggcctcagggccattccatagtgcccccctgcctcaccacctcctccccgctctcccaggctcttcttgtagaggctctccatccacctccccagtcctgttcccccagcctgggtgcccctaacttgactcttcccatctccccagcataaccaaatcttttttttttttttttttttttgagacggagtttcactctgtcgcccagactagagtgcaatggctggatctcagctcactgcaaactctgcttactgggttcaagtgattctcctgtctcagcttctgagtagctgggattacagatgcctgccaccacgcccagctaatttttttttttttttttttttttttttgtatttttagtagtgactgggtttcaccatgttggccaggctggtcttgaactcctgacctcaggtgatctgcccatctctgtctcccaaagtgctgggattacgggcgtgagccacccggctcggccgcacaaccaaatcttattaaactcaatctagtctggccctggtgactcacgcctttcggaggcagaggtgggagaatcgcttgagcccagaagtttgagaccagcctgggccacacgacaaagccccatttctacaaaaaaaaataccaaaaaaaagccagatgtggtggcatgcacctgtagtttaagctacttgggaggatgaagtgggaggactgcttgagccggggaggtggaggctggagtaaactgagatcgcgccacagaactccagtttgagcaacagatcaagaccctgcctgaaaataaatcaataaatacactcaacctaaatggaaaaaaaaaaaaaaaaa

SEQ ID NO: 40 - protein, 386 aa, Homo sapiens
MNPQIRNPMERMYRDTFYDNFENEPILYGRSYTWLCYEVKIKRGRSNLLWDTGVFRGPVLPKRQSNHRQEVYFRFENHAEMCFLSWFCGNRLPANRRFQITWFVSWNPCLPCVVKVTKFLAEHPNVTLTISAARLYYYRDRDWRWVLLRLHKAGARVKIMDYEDFAYCWENFVCNEGQPFMPWYKFDDNYASLHRTLKEILRNPMEAMYPHIFYFHFKNLLKACGRNESWLCFTMEVTKHHSAVFRKRGVFRNQVDPETHCHAERCFLSWFCDDILSPNTNYEVTWYTSWSPCPECAGEVAEFLARHSNVNLTIFTARLCYFWDTDYQEGLCSLSQEGASVKIMGYKDFVSCWKNFVYSDDEPFKPWKGLQTNFRLLKRRLREILQ

SEQ ID NO: 41 - DNA, 4706 bp, Homo sapiens
  source 1..4706: mol_type = other DNA
ttccctttgcaattgccttgggtcctgccgcacagagcggcctgtctttatcagaggtccctctgccagggggagggccccagagaaaaccagaaagagggtgagagactgaggaagataaagcgtcccagggcctcctacaccagcgcctgagcaggaagggggaggggccatgactacgaggccctgggaggtcactttagggagggctgtcctgaaacctggagcctggagcagaaagtgaaaccctggtgctccagacaaagatcttagtcgggactagccggccaaggatgaagcctcacttcagaaacacagtggagcgaatgtatcgagacacattctcctacaacttttataatagacccatcctttctcgtcggaataccgtctggctgtgctacgaagtgaaaacaaagggtccctcaaggccccgtttggacgcaaagatctttcgaggccaggtgtattcccagcctgagcaccacgcagaaatgtgcttcctctcttggttctgtggcaaccagctgcctgcttacaagtgtttccagatcacctggtttgtatcctggaccccctgcccggactgtgtggcgaagctggccgaattcctggctgagcaccccaatgtcaccctgaccatctccgccgcccgcctctactactactgggaaagagattaccgaagggcgctctgcaggctgagtcaggcaggggcccgcgtgaagattatggacgatgaagaatttgcatactgctgggaaaactttgtgtacagtgaaggtcagccattcatgccttggtacaaattcgatgacaattatgcattcctgcaccgcacgctaaaggagattctcagaaacccgatggaggcaatgtatccacacatattctacttccactttaaaaacctacgcaaagcctatggtcggaacgaaagctggctgtgcttcaccatggaagttgtaaagcaccactcacctgtctcctggaagaggggcgtcttccgaaaccaggtggatcctgagacccattgtcatgcagaaaggtgcttcctctcttggttctgtgacgacatactgtctcctaacacaaactacgaggtcacctggtacacatcttggagcccttgcccagagtgtgcaggggaggtggccgagttcctggccaggcacagcaacgtgaatctcaccatcttcaccgcccgcctctactacttctgggatacagattaccaggaggggctccgcagcctgagtcaggaaggggcctccgtggagatcatgggctacaaagattttaaatattgttgggaaaactttgtgtacaatgatgatgagccattcaagccttggaaaggactaaaatacaactttctattcctggacagcaagctgcaggagattctcgagtgaggggtctccccgggcctcatggtctgtctcctctagcctcctgctcatgttgtgcaggcctcccctccatcctggaccagctgtgcttttgcctggtcatcctgagcccctcctggcctcagggccattccatagtgctcccctgcctcaccacctcctctccgctctcccaggctcttcctgcagaggcctctttctgcctccatggctatccatccacccaccaagaccctgttccctgagcctgcatgcccctaacctgccttttcccatctccccagcataacctaatattttttttttttttttgagacggaatttcgctctgtcacccagactggagtgcaatggcttgatcttggctcactgcaaactctgcctaccaggttcaagcgattctcctgcctccgcctcccgagtagctggaattacagacgcctgccaccacgcacagctaactttttttttttttgtatttttagtagtgactgggtttcaccatgttggccaggctggtcttgaactcctgacctcaggtgatccgcctatctcagcctcccaaagtgctgggattacaggcgtgagccactggcccggcggcacaaccaaatcttattaaactcaccctaggctggccgcggtgactcatgcctataatcccccagcaatttgggaggcagaggtgagagaatcgcttgagcccaggaattcgagaccagcctgggccacatgacaaagccccatctctacaaaaaaattacaaaaaaaaaaaaaacaggtgtggtggcatgcacctgtagttgaagctacttggaaggatgaagtgggaggattgcttgagccggggaggtggaggctgcagtgaactgagatcacgtcactgaactccagtctgagcaacagatcgagaccctgcctgaaaataaatcaataaataaactcaaccgaaatgggtatgaaagttgaaatgggtatgtaagttgaaaaccagaagttttgagaaacatcctttgttaactttcatcctacaaattgggtcattcatgtcctacgcagctaaaacagagcccaggagccagggaggaaaagcagtcaggccacacaccattgctcccaaaatggacttctctgcaagcctgactcctgaaactgtgcattgtaccctgaaaccagctttatccatagcttctgcaataaatggctgtaagtcttggactccttgctataatcgcagctattcagcaatggaacctcccagttcccaacccttcctagtgcccatgggctttcccataggacaagagaacatttctccttttctttttttttttctttgaaatggagtctcgccctgtcacccaggctggagtgcaatggtgcggtctcggctcactgcaacctctgcctcccttgttcaagtgattctcctgtctcagcctcccgagtagctgggattacaggcgtccaccaccaaaccaggctaatttttgtatttttcataaaaacgggtttcatcatgtttcccaggctggtcttatttttattttattttttgagatggagtcttgctctgttgcccaggctggggtgcagtggtgcaatctgggttcactgcagcctctgccgcctgagttcaagctattttcctacctaagcctcccaagtagctgggattacatgcgcgtgccaccacgcctagctaatttttgtgtttttagtagagacggggtttcaacatcttgaccaggctggtcttgaactcctgacctcgtgatccacccgtctcggcctcccaaagtgctgggattacaggcgtgagccacctggccaggcttaggctggtcttaaactcctgacctcaagtgatccaacctccttggcctcccaaattgctgggattgctggtgtgagccacagcgcctagcccatttctccttttaataggacctgttgctgtctctgttctcccaacatggtgaacaccacccggactgcgtgtatgtcccaaattacaattctttctttgcaaatgaaatgtgaaatttagaggcccttctccacactttaaatttgacttgacattttctaggcagatataagttattagagaatgagattctctataaaaatgatcccttcatgctgtggcctccacagaagatgccctgggccaggtgcccacatgaataatgcgggccacaggcaggcatttattttctcacagatatggaggctacaagtccaaggtggaggggtcggcggggttgtttgctctgaggccgctcctcctggatggcagggatcccttctggctgtgtcctctgtggcctttcctctatgaacctgtactgtacctctggggtctctctgcttccaaatatcttttttttttttttcagacagttttgctcttgttttctaggctggagtgcaatggcacaatctcagctcactgcaacctctgccttccgagttcaagcgattctcgtgcctcagcctcctgagtagctgggactacaggcgtgtgccaccacgcctggctaattttgtagttttagtagagacggggtttctccatgttgctcaggctggtcttgaactcatgagctcaggcgatccactctcctcagcctcccaaagtgctgggattacagatataagccaccatacacaacttttttttttttttgagatggagtttcactctgttgcccaggctggagtgctaaatagcagaatcactgctcactgcaacctctgcctgctgggttcaagcaattctcccacctcagcctcctgagtagctgggattacagatgcccagaaccaatctctgctaatttttctattttttagtagagatggggtttcactgaggaaggagaccacctctctcattgtctcctatttcagaaggaagcaaaaagttagaaagatgcagaagtaagatcaatggccagactgtttggcgctgctacctgggcctggtagttaaagatcaactcctgacctgaccgcttgttttatctaaagattccagacattgtatgaggaagcattgtgaaactttctggtctgttctgctagcccccaccactgatgcatgtagccccccagtcacgtagcccacgcttgcacaatctatcacgaccctttcacgtggaccccttagaattgtaagcccttaaaagggccagggacttcttcagggagctccaatcttcagatgcaagtctgtcaacgctcccagctgattaaagcctcttccttcctaaaaaaaaaaaaaaaaaa

SEQ ID NO: 42 - protein, 373 aa, Homo sapiens
MKPHFRNTVERMYRDTFSYNFYNRPILSRRNTVWLCYEVKTKGPSRPRLDAKIFRGQVYSQPEHHAEMCFLSWFCGNQLPAYKCFQITWFVSWTPCPDCVAKLAEFLAEHPNVTLTISAARLYYYWERDYRRALCRLSQAGARVKIMDDEEFAYCWENFVYSEGQPFMPWYKFDDNYAFLHRTLKEILRNPMEAMYPHIFYFHFKNLRKAYGRNESWLCFTMEVVKHHSPVSWKRGVFRNQVDPETHCHAERCFLSWFCDDILSPNTNYEVTWYTSWSPCPECAGEVAEFLARHSNVNLTIFTARLYYFWDTDYQEGLRSLSQEGASVEIMGYKDFKYCWENFVYNDDEPFKPWKGLKYNFLFLDSKLQEILE

SEQ ID NO: 43 - DNA, 822 bp, Homo sapiens
  source 1..822: mol_type = other DNA
ttccctttgcaattgccttgggtcctgccgcacagagcggcctgtctttatcagaggtccctctgccagggggagggccccagagaaaaccagaaagagggtgagagactgaggaagataaagcgtcccagggcctcctacaccagcgcctgagcaggaagggggaggggccatgactacgaggccctgggaggtcactttagggagggctgtcctgaaacctggagcctggagcagaaagtgaaaccctggtgctccagacaaagatcttagtcgggactagccggccaaggatgaagcctcacttcagaaacacagtggagcgaatgtatcgagacacattctcctacaacttttataatagacccatcctttctcgtcggaataccgtctggctgtgctacgaagtgaaaacaaagggtccctcaaggccccgtttggacgcaaagatctttcgaggccaggtgcccaggtctttcatcagagccccatttcaagtgctcagtagcccctttggccagtgcgccccaccacatgggacagcgcaggtccagtggcctccccagctgaccgcaggcagggaacaaggcagaccctagagggccaggccacagcaggggctgaggatgcctggtgaatggatgcctgggagaatggatgccagaattcacgcatgaggctctgaacagggctgggaaaacttccaaacgaagggaagctcatgtcttggtgcactttgtgatgatgcttcaacagcaggactgagatggggacatttacaataaacagaaatgtatgggctcgaaaaaaaaaaaaaaaaaa

SEQ ID NO: 44 - protein, 101 aa, Homo sapiens
MKPHFRNTVERMYRDTFSYNFYNRPILSRRNTVWLCYEVKTKGPSRPRLDAKIFRGQVPRSFIRAPFQVLSSPFGQCAPPHGTAQVQWPPQLTAGREQGRP

SEQ ID NO: 45 - DNA, 1848 bp, Homo sapiens
  source 1..1848: mol_type = other DNA
gtgctctgctggctcagcctggtgtggacccacctcccgggcgctggctgcaatgactttctctttccctttgcaattgccttgggtcctgccgcacagagcggcctgtctttatcagaggtccctctgccagggggagggccccagagaaaaccagaaagagggtgagagactgaggaagataaagcgtcccagggcctcctacaccagcgcctgagcaggaagcgggaggggccatgactacgaggccctgggaggtcactttagggagggctgtcctaaaaccagaagcttggagcagaaagtgaaaccctggtgctccagacaaagatcttagtcgggactagccggccaaggatgaagcctcacttcagaaacacagtggagcgaatgtatcgagacacattctcctacaacttttataatagacccatcctttctcgtcggaataccgtctggctgtgctacgaagtgaaaacaaagggtccctcaaggccccctttggacgcaaagatctttcgaggccaggtgtattccgaacttaagtaccacccagagatgagattcttccactggttcagcaagtggaggaagctgcatcgtgaccaggagtatgaggtcacctggtacatatcctggagcccctgcacaaagtgtacaagggatatggccacgttcctggccgaggacccgaaggttaccctgaccatctttgttgcccgcctctactacttctgggacccagattaccaggaggcgcttcgcagcctgtgtcagaaaagagacggtccgcgtgccaccatgaagatcatgaattatgacgaatttcagcactgttggagcaagttcgtgtacagccaaagagagctatttgagccttggaataatctgcctaaatattatatattactgcacatcatgctgggggagattctcagacactcgatggatccacccacattcactttcaactttaacaatgaaccttgggtcagaggacggcatgagacttacctgtgttatgaggtggagcgcatgcacaatgacacctgggtcctgctgaaccagcgcaggggctttctatgcaaccaggctccacataaacacggtttccttgaaggccgccatgcagagctgtgcttcctggacgtgattcccttttggaagctggacctggaccaggactacagggttacctgcttcacctcctggagcccctgcttcagctgtgcccaggaaatggctaaattcatttcaaaaaacaaacacgtgagcctgtgcatcttcactgcccgcatctatgatgatcaaggaagatgtcaggaggggctgcgcaccctggccgaggctggggccaaaatttcaataatgacatacagtgaatttaagcactgctgggacacctttgtggaccaccagggatgtcccttccagccctgggatggactagatgagcacagccaagacctgagtgggaggctgcgggccattctccagaatcaggaaaactgaaggatgggcctcagtctctaaggaaggcagagacctgggttgagcctcagaataaaagatcttcttccaagaaatgcaaacaggctgttcaccaccatctccagctgatcacagacaccagcaaagcaatgcactcctgaccaagtagattcttttaaaaattagagtgcattactttgaatcaaaaatttatttatatttcaagaataaagtactaagattgtgctcaatacacagaaaagtttcaaacctactaatccagcgacaatttgaatcggttttgtaggtagaggaataaaatgaaatactaaatctttctgtaaaaaaaaaaaaaaa

SEQ ID NO: 46 - protein, 384 aa, Homo sapiens
MKPHFRNTVERMYRDTFSYNFYNRPILSRRNTVWLCYEVKTKGPSRPPLDAKIFRGQVYSELKYHPEMRFFHWFSKWRKLHRDQEYEVTWYISWSPCTKCTRDMATFLAEDPKVTLTIFVARLYYFWDPDYQEALRSLCQKRDGPRATMKIMNYDEFQHCWSKFVYSQRELFEPWNNLPKYYILLHIMLGEILRHSMDPPTFTFNFNNEPWVRGRHETYLCYEVERMHNDTWVLLNQRRGFLCNQAPHKHGFLEGRHAELCFLDVIPFWKLDLDQDYRVTCFTSWSPCFSCAQEMAKFISKNKHVSLCIFTARIYDDQGRCQEGLRTLAEAGAKISIMTYSEFKHCWDTFVDHQGCPFQPWDGLDEHSQDLSGRLRAILQNQEN

SEQ ID NO: 47 - DNA, 1164 bp, Homo sapiens
  source 1..1164: mol_type = other DNA
tgacttttgggagagctgaccttttgtgacttttgggagagctgccaaaagtgaaacttagtgcctcagacaagcaggggcaagtctgctaaggaagctgtggccagaagcacagatcagaaacacgatggctctgttaacagccgaaacattccgcttacagtttaacaacaagcgccgcctcagaaggccttactacccgaggaaggccctcttgtgttaccagctgacgccgcagaatggctccacgcccacgagaggctactttgaaaacaagaaaaagtgccatgcagaaatttgctttattaacgagatcaagtccatgggactggacgaaacgcagtgctaccaagtcacctgttacctcacgtggagcccctgctcctcctgtgcctgggagctggttgacttcatcaaggctcacgaccatctgaacctgggcatcttcgcctcccgcctgtactaccactggtgcaagccccagcagaaggggctgcggcttctgtgtggatcccaggtcccggtggaggtcatgggcttcccagagtttgctgactgctgggaaaactttgtggaccacgagaaaccgctttccttcaacccctataagatgttagaggagctagataaaaacagtcgagccataaagcgacggcttgagaggataaagattccaggggtacgtgcgcagggtcgttacatggatatattgtgtgatgctgaggtctgagtcacccaatctactggaacatagcacccaatagcagtcctgaagtgtggatgttttagagaatgacttaagaagtttgcagcttggacccgtatcccactcattatcaagaagcaactcaagatgactttccctggggcatgtcagttgcctcatagcctgctggtcctgtaagcaagcactaagctccacagtgccagttccttgccccaacctggccccatccaagtacagaagaccttcctttcctcctttttccatattgctttctgttctaagtgggtgaataattttataattgaaaaaataaagataaagtctgtaaatccagccgggtacggtggctcacgcctgtaatcctagcactttgggaggctgagatgctcggccaataaatttctattgtttatgaaaaaaaaaaaaaaaaaaaaaaa

SEQ ID NO: 48 - protein, 200 aa, Homo sapiens
MALLTAETFRLQFNNKRRLRRPYYPRKALLCYQLTPQNGSTPTRGYFENKKKCHAEICFINEIKSMGLDETQCYQVTCYLTWSPCSSCAWELVDFIKAHDHLNLGIFASRLYYHWCKPQQKGLRLLCGSQVPVEVMGFPEFADCWENFVDHEKPLSFNPYKMLEELDKNSRAIKRRLERIKIPGVRAQGRYMDILCDAEV

SEQ ID NO: 49 - DNA, 2786 bp, Homo sapiens
  source 1..2786: mol_type = other DNA
agtgttcttagaggtagccacacctcagcaagggctttctaacttggaacagcaaagttcaaatgaacgaatagacacaaactgtgaaagagtcctgaacccattacaaggtgaatctttacagcacaattgccttctgaagctacttgaaacagggcatgggacttaaaggagaaaaaaaaggaaatggaccatctcttctaacacctggaatgtgatccaagcctggagacaaattgatggtttgcagctagaagacagtaaatctagcaatggagcccatatatgaggagtacctagcaaatcatggaacaatagtaaaaccatattactggctaagcttctctctcgattgctctaattgtccttaccatattcgaacaggtgaagaagcaagagtttccctcacagaattttgtcagatttttggattcccttatgggacaacatttcctcaaacaaaacacctcacattttatgaactaaaaacttcttctggtagcctggtgcaaaagggccatgctagcagttgcactgggaattatatccatccagaatcaatgctctttgaaatgaatggttatcttgactcagccatatacaataatgacagcatcaggcatatcattctgtattccaacaactccccttgtaatgaagctaaccactgctgcatcagcaaaatgtataatttcctgattacgtatccaggcatcactcttagtatttatttttctcagctctatcatactgagatggactttcctgcctcagcatggaaccgcgaagctctccggagcctggccagcttatggccgcgggttgttttgagtccaataagtggtgggatctggcattctgttctccacagctttataagtggtgtctcaggatcacatgtttttcagcccattttaactgggagagcactggctgacaggcacaacgcatatgaaatcaatgccataacaggcgtaaaaccttacttcactgatgttcttctccagacaaaaaggaatccaaacacaaaagctcaggaggctttagagagctaccccttaaacaatgcctttcctggacagttttttcaaatgccgagtggacaactccaacccaacctacctccagacctcagggctcctgttgtttttgtgctagtgcctctcagggacttaccaccaatgcatatgggccaaaacccaaataaacccaggaatatcgtaaggcacttaaatatgcctcaaatgtcattccaggaaaccaaggaccttggaaggcttcccactggaaggtcagtggagatagtggaaatcacagaacagtttgcaagtagcaaggaggcagatgaaaagaagaagaagaaagggaagaaataaaatctacattcctacagcatgaaaccaattaccaagggacttattagataggcaataaaaatctggaaactttctctctcagggctgagccaagatggagataaactgatgcactaaaagcaaaccttgaattatttaccatatcaactattataaagttacactattttaaacataatccaaaatgtttccataagcaacttgatctctctcctttattttgaaagtggcagtatcaaaatggaaccattagcagcttgctaatattttgatgcagaaaaggaatctcattattctattttactcacatcatctttccactacaaaagcccatggacaaaatagctacctaaatttgtttcggtaaaaaatttactgtaaaattgaaaaacagcctgaagctactgacattaagtgctgtttgcaggagcaaaccagtgattttctattaattgttaatcatggacagaagattttaggcagctcatttttttctaaagtgattcaaaatagttttgtgtgggaaaaagctcttctgctgattacatatagctataaagattaaaacctagccacctgctcagtttgatcttccccagctcagacctctcccttctgagcagatgatagaggtaagatgatacatgagaacctccaatggctcctgtcatcacatactgtataaaatccaagttcctcttactggctttcaaggatcctcctttaacttcctgttgccttgtctcatcagcaagatcataagtacctacaggtcaagcactgtctctcccttctctttagcttttcccttaggatctagcacattacccagcaaaatgtgagtagcaaggctgaaatgacatctcaataacttcaccaatgattgtaactcagcatcccttctccatcccagctgaaagcctgcttcaccatcctgcaagagatgttttttctttttgttagcatccattcccctttctaatgcagctcccaatgcataatgtgtgttttacttccctctttctgactccatcctcgtcaccagtgaatatttgtaccttctcttaaaatcttcaaaacacacttctaggaagtgctccttgattaatcctctaagcacttaaatgaaagcactttgcttatgcttatttcacatttgctgccacttgcctctttgaaatatgtaaagatacattacaagaaaaatgtacattttctcgtaacttgctattaatttttttctttatatatatatataaagtaatcctaagctaataaaagagtttgccatgacattaaaacatttaatactccataagggaggaggagaaagaaaaacatattttaggaatagcaattagtattgtggttaaaataaacatgaataaaaattttaaaacaaaaaaaaaa

SEQ ID NO: 50 - protein, 367 aa, Homo sapiens
MEPIYEEYLANHGTIVKPYYWLSFSLDCSNCPYHIRTGEEARVSLTEFCQIFGFPYGTTFPQTKHLTFYELKTSSGSLVQKGHASSCTGNYIHPESMLFEMNGYLDSAIYNNDSIRHIILYSNNSPCNEANHCCISKMYNFLITYPGITLSIYFSQLYHTEMDFPASAWNREALRSLASLWPRVVLSPISGGIWHSVLHSFISGVSGSHVFQPILTGRALADRHNAYEINAITGVKPYFTDVLLQTKRNPNTKAQEALESYPLNNAFPGQFFQMPSGQLQPNLPPDLRAPVVFVLVPLRDLPPMHMGQNPNKPRNIVRHLNMPQMSFQETKDLGRLPTGRSVEIVEITEQFASSKEADEKKKKKGKK

SEQ ID NO: 51 - DNA, 1271 bp, Arabidopsis thaliana
  source 1..1271: mol_type = other DNA
gcgtaatgtagtttcctacgttgttgtatctataaatagtttgtttgtccagcatccatttcataattcctcatttcccggatctctcccatctaaaaataacccgacccatttacgcgacccaaaccggatcaacccgcaatggataagccaagcttcgtaatccaatccaaagaagcagaatccgccgcgaaacaactcggcgtttccgtcattcagctcctcccgtcgctagtcaaaccagcacaatcctacgctcgaactccgatttcgaaattcaacgtcgcagtcgtcggactcggatcatcaggtcggatcttcttaggcgtcaatgtcgaattcccaaatctccctctccaccactcaatccacgccgaacagttcctcgtcaccaatctcacactcaacggtgaacgtcatctcaatttcttcgccgtctccgccgcaccatgtggccattgccgtcaattcctccaagaaattcgcgacgcacctgaaatcaaaatccttatcaccgatccaaacaactccgccgattccgattccgccgccgattcagacggattcttacgtctcggaagcttcttgccacacagattcggtcccgacgatcttctcgggaaagatcatcctcttcttctcgaatctcacgataaccatctcaaaatctcagatctggattcgatttgtaacggaaacaccgattcatccgccgatttgaaacaaacggctttagcggcggcgaatagatcgtacgcgccgtatagtttatgtccatcgggagtttcgctggtggattgtgacgggaaagtgtacagaggttggtatatggaatcggcggcgtataatcctagtatgggaccagtacaggcggcgttggttgattatgtggctaatggtggtggaggaggatacgagaggatcgtcggagcggttctggtggagaaagaagatgcggtggtgaggcaagagcacacggcgaggttgttattagagactatatcgccgaaatgcgaattcaaagtgtttcattgctatgaagcttagagaatcatttaattatccgaaaaataaaggaattttaaaaaaaaaggaataaaaagaaaatgagactttttaattcttaaaagtgttgaaattgacggaaaaaaaaaaagtgttgaaattgatgtgatggataacacatagagaataagaaaagtttatatttttaaatttgttaaaaatgtttattgtattaaaaaattaaatttaatacaattattatgttt

SEQ ID NO: 52 - protein, 301 aa, Arabidopsis thaliana
MDKPSFVIQSKEAESAAKQLGVSVIQLLPSLVKPAQSYARTPISKFNVAVVGLGSSGRIFLGVNVEFPNLPLHHSIHAEQFLVTNLTLNGERHLNFFAVSAAPCGHCRQFLQEIRDAPEIKILITDPNNSADSDSAADSDGFLRLGSFLPHRFGPDDLLGKDHPLLLESHDNHLKISDLDSICNGNTDSSADLKQTALAAANRSYAPYSLCPSGVSLVDCDGKVYRGWYMESAAYNPSMGPVQAALVDYVANGGGGGYERIVGAVLVEKEDAVVRQEHTARLLLETISPKCEFKVFHCYEA

SEQ ID NO: 53 - DNA, 2433 bp, synthetic construct
  source 1..2433: mol_type = other DNA
atgcagagggtgaacatgatcatggctgagagccctggcctgatcaccatctgcctgctgggctacctgctgtctgctgaatgtacaggtttgtttccttttttataatacattgagtatgcttgccttttagatatagaaatatctgattctgtcttcttcactaaattttgattacatgatttgacagcaatattgaagagtctaacagccagcacccaggttggtaagtactggttctttgttagctaggttttcttcttcttcacttttaaaactaaatagatggacaatgcttatgatgcaataaggtttaataaacactgttcagttcagtatttggtcatgtaattcctgttaaaaaacagtcatctccttggtttaaaaaaattaaaagtgggaaaacaaagaaatagcagaatatagtgaaaaaaaataaccacagtatttttgtttggacttaccactttgaaatcaaattagaggaaggcatacagggaagaaattatcccatttggacaaacagcatgttctcacagtaagcacttatcacacttacttgtcaacttttagaatcaaatctagtagctgacagtaccaggatcaggggtgccaaccctaagcacccccagaaagctgactggccctgtggttcccactccagacatgatgtcagctgtgaaatccacctccctggaccataattaggcttctgttcttcaggagacatttgttcaaagtcatttgggcaaccatattctgaaaacagcccagccagggtgatggatcactttgcaaagatcctcaatgagctattttcaagtgatgacaaagtgtgaagttaagggctcatttgagaactttctttttcatccaaagtaaattcaaatatgattagaaatctgaccttttattactggaattctcttgactaaaagtaaaattgaattttaattcctaaatctccatgtgtatacagtactgtgggaacatcacagattttggctccatgccctaaagagaaattggctttcagattatttggattaaaaacaaagactttcttaagagatgtaaaattttcatgatgttttcttttttgctaaaactaaagaattattcttttacatttcagtttttcttgatcatgaaaatgccaacaaaattctgaatagaccaaagaggtataactctggcaagcttgaagagtttgtacaggggaatctggagagagagtgtatggaagagaagtgcagctttgaggaagccagagaagtgtttgaaaatacagagagaacaactgaattttggaagcagtatgtggatggtgatcaatgtgagagcaatccctgcttgaatggggggagctgtaaagatgacatcaacagctatgaatgttagtgtccctttggatttgaggggaaaaactgtgagcttgatgtgacctgtaatatcaagaatggcaggtgtgagcaattttgcaagaattctgctgataacaaagtggtctgtagctgcactgagggatataggctggctgaaaaccagaagagctgtgaacctgcagtgccttttccctgtgggagagtgtctgtgagccaaaccagcaagctgactagggctgaagcagtctttcctgatgtagattatgtgaatagcactgaggctgagacaatccttgacaatatcactcagagcacacagagcttcaatgacttcaccagggtggtaggaggggaggatgccaagcctgggcagttcccctggcaggtagtgctcaatggaaaagtggatgccttttgtggaggttcaattgtaaatgagaagtggattgtgactgcagcccactgtgtggaaactggagtcaagattactgtggtggctggagagcacaatattgaggaaactgagcacactgagcagaagaggaatgtgatcaggattatcccccaccacaactacaatgctgctatcaacaagtacaaccatgacattgccctcctggaactggatgaacccctggtcttgaacagctatgtgacacccatctgtattgctgataaagagtacaccaacatcttcttgaaatttgggtctggatatgtgtctggctggggcagggtgttccataaaggcaggtctgccctggtattgcagtatttgagggtgcctctggtggatagagcaacctgcttgaggagcaccaagtttacaatctacaacaatatgttctgtgcagggttccatgaaggtggtagagacagctgccagggagattctgggggtccccatgtgactgaggtggagggaaccagcttcctgactgggattatcagctggggtgaggagtgtgctatgaagggaaagtatgggatctacacaaaagtatccagatatgtgaactggattaaggagaaaaccaagctgacttga

SEQ ID NO: 54 - RNA, 659 nt, synthetic construct
  source 1..659: mol_type = other RNA
gagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgccatcagtcgccggtcccaagcccggataaaatgggagggggcgggaaaccgcctaaccatgccgactgatggcagaaaaaaaaaattatcagcagaattcttgcaaaattgctcacacctgccattcttgatattacaggtcacatcaagctcacagtttttcccctcaaatccaaagggacaccaacattcatagctgttgatgtcatctttacagctccccccattcaagcagggattgctctcacattgatcaccatccacatactgcttccaaaattcagtaaaaaaaaaactgccatcagtcggcgtggactgtagaacactgccaatgccggtcccaagcccggataaaagtggagggtacagtccacgcttttttcgtactgagtcgcccagtctcagata

SEQ ID NO: 55 - RNA, 659 nt, synthetic construct
  source 1..659: mol_type = other RNA
gagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgccatcagtcgccggtcccaagcccggataaaatgggagggggcgggaaaccgcctaaccatgccgactgatggcagaaaaaaaaaattatcagcagaattcttgcaaaattgcgtgaacctgccattcttgaaataacaggtcacatcaagtgagcagtttttcccctcaaatccaaagggacaccaacatatgaagctgttgatgtcataaagacagctccccccattcttgcagggattgctctcacattgatcaccatccacatactgcttccaaaattcagtaaaaaaaaaactgccatcagtcggcgtggactgtagaacactgccaatgccggtcccaagcccggataaaagtggagggtacagtccacgcttttttcgtactgagtcgcccagtctcagata

(19) United States
(12) Patent Application Publication
(10) Pub. No.: US 2026/0124230 A1
(43) Pub. Date: May 7, 2026

(54) GENE EDITING FOR CONTROLLED EXPRESSION OF EPISOMAL GENES

(71) Applicant: Spark Therapeutics, Inc., Philadephia, PA (US)

(72) Inventors: Sean Armour, Holland, PA (US); Ali Nahvi, Needham, MA (US); Federico Mingozzi, Philadelphia, PA (US); Daniel Cohen, Philadelphia, PA (US); Yahui Lan, Philadelphia, PA (US)

(21) Appl. No.: 19/119,442

(22) PCT Filed: Oct. 13, 2023
(86) PCT No.: PCT/US2023/076855

§ 371 Date: Apr. 9, 2025

Related U.S. Application Data
(60) Provisional application No. 63/379,512, filed on Oct. 14, 2022.

(51) Int. Cl. A61K 31/7105; A61K 38/46; A61K 38/50; A61K 48/00; C12N 15/86

(52) U.S. Cl. CPC A61K 31/7105; A61K 38/465; A61K 38/50; A61K 48/0033; C12N 15/86; C12Y 305/04004; C12Y 305/04005; C12N 2750/14143

(57) ABSTRACT

Disclosed herein are methods for regulating expression of a gene located on an episomal vector in a subject in need thereof. In particular, methods include administering to the subject one or more gene editing agents that modify a region of the gene to thereby regulate the expression of the gene. Also disclosed are methods of administering to the subject a base editor system that effects a base alteration in a region of the gene or a region of an mRNA transcript of the gene to thereby regulate the expression of the gene.

### CROSS REFERENCE TO RELATED APPLICATION

This application claims priority to U.S. Provisional Patent Application 63/379,512, filed Oct. 14, 2022, the disclosure of which is incorporated herein by reference.

### REFERENCE TO SEQUENCE LISTING SUBMITTED ELECTRONICALLY

This application contains a sequence listing, which is submitted electronically. The content of the electronic sequence listing (065830-15WO1 Sequence Listing.xml; size: 130 KB; and date of creation: Nov. 22, 2023) is herein incorporated by reference in its entirety.

### FIELD OF THE INVENTION

The invention relates to the field of gene therapy. In particular, it relates to controlling gene expression from an episomal vector by utilizing gene editing agents, such as base editing systems.

### BACKGROUND OF THE INVENTION

Gene therapy in its current design is an irreversible process. It cannot be stopped in case of unwanted side effects, nor can expression levels of therapeutics be adjusted to individual patient's needs. Adeno-associated viral (AAV) vector-mediated gene therapy holds great potential for future medical applications. However, to facilitate safer and broader applicability and to enable patient-centric care, therapeutic protein expression should be controllable. For example, it has been shown in certain diseases that gene therapy in which genes become overexpressed may be toxic (Payne, Mol Ther Methods Clin Dev. 2022 March 4:25:1-2.; Palmieri et al., Front Neurosci. 2023 May 25:17:1172805). Conversely, in other diseases, such as Huntingtin's Disease, too much gene repression may be toxic (Jung et al., Hum Mol Genet. 2021 Apr. 26; 30(3-4):135-148; Wang et al., Proc Natl Acad Sci USA. 2016 Mar. 22; 113(12):3359-64; Murthy et al., PLoS Genet. 2019 March; 15(3): e1007765). In other cases, AAV gene therapy has been shown to have a current risk/benefit profile that is low. For example, in diseases with existing treatments, or diseases where patient outcomes are better or have less serious consequences (Evan et al., Curr Opin Rheumatol. 2023 Jan. 1; 35(1):37-43; Ishikawa et al., Circ Res. 2018 Aug. 17; 123(5):601-613). Thus, a method of regulating gene therapy in such diseases is needed.

Base editing is a genome editing method that directly generates precise point mutations in genomic DNA or in cellular RNA without directly generating double-strand breaks (DSBs), requiring a DNA donor template or relying on cellular homology directed repair. Since base editors do not normally create DSBs, they minimize the formation of DSB-associated byproducts (Komor, A. C. et al, (2017) Improved base excision repair inhibition and bacteriophage Mu Gam protein yields C:G-to-T:A base editors with higher efficiency and product purity, Sci Adv 3, and Rees, H. A. et al., (2017) Improving the DNA specificity and applicability of base editing through protein engineering and protein delivery, Nat. Commun. 8, 15790). Base editors (BEs) are typically fusions of a Cas (“CRISPR-associated”) domain and a nucleobase modification domain. Two major types of base editors have been developed and widely used. The first type includes cytosine base editors (CBEs), which were first reported in 2016 (Komor et al., 2016; (2016) Targeted AID-mediated mutagenesis (TAM) enables efficient genomic diversification in mammalian cells. Nat. Methods, 13, 1029-1035; Nishida et al., (2016) Targeted nucleotide editing using hybrid prokaryotic and vertebrate adaptive immune systems. Science, 353, aaf8729). The second type includes adenine base editors (ABEs) that were first described in 2017 (Gaudelli et al., (2017) Programmable base editing of A*T to G*C in genomic DNA without DNA cleavage. Nature, 551, 464-471). Both CBEs and ABEs were based on the CRISPR-Cas9 system, utilizing cytidine deaminases and adenine deaminases to confer C-to-T and A-to-G base transition changes in the editing windows respectively. CBEs can convert four codons (CGA, CAG, GAG, GAA and TGG) into stop codons (TGA, TAG, TAA) (Kuscu et al., (2017) CRISPR-STOP: gene silencing through base-editing-induced nonsense mutations. Nat. Methods, 14, 710-712; Molla and Yang, (2019) CRISPR/Cas-mediated base editing: technical considerations and practical applications. Trends Biotechnol. 37, 1121-1142). Hence, CBEs can be used for knocking out protein-coding genes by introducing premature stop codons.

The present disclosure provides methods of regulating expression of a gene located on an episomal vector in a subject. In particular, the present disclosure provides a method of regulating expression of a gene located on an episomal vector utilizing a base editor system.

### BRIEF SUMMARY OF THE INVENTION

The disclosure provides methods of regulating expression of a gene located on an episomal vector in a subject in need thereof, comprising administering to the subject one or more gene editing agents that modify a region of the gene to thereby regulate the expression of the gene, wherein the region of the gene comprises one or more of a promoter, an enhancer, a silencer, or an insulator, a premature termination codon convertible to an amino acid codon via the modification of the region, or an amino acid codon convertible to a premature termination codon via the modification of the region.

In certain embodiments, the one or more gene editing agents comprise a guide RNA that is complementary to the region of the gene and a Cas protein or a derivative of the Cas protein.

In certain embodiments, the Cas protein is Cas9, such as Staphylococcus aureus Cas9 (SaCas9), Streptococcus thermophilus 1 Cas9 (St1Cas9), a modified Streptococcus pyogenes Cas9 (SpCas9); a CpF1; a CasX; a CasY; a C2c1; a C2c2; a C2c3; or a variant thereof.

In certain embodiments, wherein the one or more gene editing agents further comprise a donor nucleic acid having at least one nucleotide change relative to the region of the gene and capable of integrating into the region of the gene to modify the region.

In certain embodiments, the one or more gene editing agents are encoded by one or more nucleic acid molecules administered to the subject, preferably the one or more gene editing agents are encoded by a RNA molecule, particularly an mRNA molecule, administered to the subject.

Also provided is a method of regulating expression of a gene located on an episomal vector in a subject in need thereof, comprising administering to the subject a base editor system that effects a base alteration in a region of the gene or a region of an mRNA transcript of the gene to thereby regulate the expression of the gene.

In certain embodiments, the region of the gene comprises one or more of a promoter, an enhancer, a silencer or an insulator, or the region of the gene or the region of the mRNA transcript comprises a premature termination codon convertible to an amino acid codon via the base alteration, or an amino acid codon convertible to a premature termination codon via the base alteration.

In certain embodiments, the base editor system comprises: (a) a ribonucleic acid complementary to the region of the gene; and (b) a base editor comprising a polynucleotide programmable DNA binding domain and an adenosine deaminase domain or a cytidine deaminase domain, wherein the polynucleotide programmable DNA binding domain in conjunction with the ribonucleic acid binds to the region of the gene to effect the base alteration.

In certain embodiments, the polynucleotide programmable DNA binding domain comprises a nuclease inactive variant of a Cas protein or a nickase variant of a Cas protein.

In certain embodiments, the base editor further comprises a uracil binding protein, such as a uracil glycosylase inhibitor (UGI) domain that inhibits a uracil-DNA glycosylase.

In certain embodiments, (i) the cytidine deaminase domain is selected from the group consisting of the apolipoprotein B mRNA-editing enzyme, catalytic polypeptide-like (APOBEC) family of deaminases, such as APOBEC1, APOBEC2, APOBEC3A, APOBEC3B, APOBEC3C, APOBEC3D/E, APOBEC3F, APOBEC3G, APOBEC3H, or APOBEC4; activation-induced cytidine deaminase (AID), such as activation induced cytidine deaminase (AICDA); cytosine deaminase 1 (CDA1) or CDA2; or cytosine deaminase acting on tRNA (CDAT), and (ii) the adenosine deaminase is selected from the group consisting of adenosine deaminase 1 (ADA1) and ADA2.

In certain embodiments, the base editor system comprises: (a) a ribonucleic acid complementary to the region of the mRNA transcript; and (b) a base editor comprising a polynucleotide programmable RNA binding domain and an adenosine deaminase domain or a cytidine deaminase domain, wherein the polynucleotide programmable RNA binding domain in conjunction with the ribonucleic acid binds to the region of the mRNA transcript to effect the base alteration.

In certain embodiments, the polynucleotide programmable RNA binding domain comprises a nuclease inactive variant of Cas13 or a nickase variant of Cas13. In particular embodiments, the Cas13 is Cas13a and Cas13b.

In certain embodiments, i) the cytidine deaminase domain is selected from the group consisting of the apolipoprotein B mRNA-editing enzyme, catalytic polypeptide-like (APOBEC) family of deaminases, such as APOBEC1, APOBEC2, APOBEC3A, APOBEC3B, APOBEC3C, APOBEC3D/E, APOBEC3F, APOBEC3G, APOBEC3H, or APOBEC4; activation-induced cytidine deaminase (AID), such as activation induced cytidine deaminase (AICDA); cytosine deaminase 1 (CDA1) or CDA2; or cytosine deaminase acting on tRNA (CDAT), and (ii) the adenosine deaminase is selected from the group consisting of adenosine deaminase acting on RNA 1 (ADAR1), ADAR2, ADAR3; adenosine deaminase acting on tRNA 1 (ADAT1), ADAT2, ADAT3; and naturally occurring or engineered tRNA-specific adenosine deaminase (TadA).

In certain embodiments, the ribonucleic acid is a guide RNA.

In certain embodiments, the base editor system or a component thereof is encoded by one or more nucleic acid molecules administered to the subject, preferably the ribonucleic acid and the base editor are encoded by one or more RNA molecules, such as one or more mRNA molecules, administered to the subject.

In certain embodiments, the one or more nucleic acid molecules, such as the one or more mRNA molecules, are administered to the subject with a lipid nanoparticle (LNP), a peptide cage, or a polymer nanoparticle.

In certain embodiments, the base alteration results in a conversion of the amino acid codon to the premature stop codon, preferably upstream of a splice junction, to thereby down-regulate the expression of the gene.

In certain embodiments, wherein the base alteration results in the conversion of a CGA, CAG, or TGG codon to a premature TGA, TAG, or TAA stop codon, respectively, and the base editor comprises the cytidine deaminase domain, preferably, the CAG codon is located in proximity to the 5′-end of the gene.

In certain embodiments, the base alteration results in a conversion of a premature stop codon to an amino acid codon to thereby up-regulate the expression of the gene.

In certain embodiments, wherein the base alteration results in the conversion of a premature UAG, UAA, or UGA stop codon to a CAG, CAA, or CGA, respectively, and the base editor comprises the adenosine deaminase domain, preferably, the premature UAG stop codon is located in proximity to the 5′-end of the gene.

In certain embodiments, further comprising administering to the subject the episomal vector comprising the gene. In certain embodiments, the episomal vector is a nonviral vector, such as a plasmid, or a viral vector, such as an adeno-associated viral (AAV) vector, a lentivirus vector, or an adenovirus vector. In certain embodiments, wherein the episomal vector is an AAV vector.

In certain embodiments, the subject is a human, such as a human subject suffering from Hereditary angioedema, Pompe disease, hemophilia A, hemophilia B, Fabry disease, Huntington disease, Parkinson's disease, Alzheimer's disease, a synucleinopathy, epilepsy, neuropathic pain, wet macular degeneration, Usher 1F, Usher 1B, glaucoma, Leber congenital amaurosis, and Stargardt disease.

Also provided is a method of regulating expression of a gene located on an episomal vector in a subject in need thereof, comprising administering to the subject an editing agent that effects an alteration in a region of an mRNA transcript of the gene to thereby regulate the expression of the gene.

In certain embodiments, the editing agent effects a base alteration in the region of the mRNA transcript of the gene.

In certain embodiments, the alteration in a region of an mRNA transcript of the gene alters the stability of the mRNA transcript, the initiation or level of the translation of the mRNA transcript, the stability and/or activity of the translated protein.

In certain embodiments, the region of the mRNA transcript comprises a premature termination codon convertible to an amino acid codon via the base alteration, or an amino acid codon convertible to a premature termination codon via the base alteration.

In certain embodiments, the base alteration (a) is within a microRNA targeting site or a toe-hold switch site, or (b) induces a ribosomal frameshift or alters a codon encoding an amino acid residue critical to function and/or structure of an encoded protein.

In certain embodiments, the editing agent comprises a targeting ribonucleic acid complementary to the region of the mRNA transcript.

In certain embodiments, the targeting ribonucleic acid is linear.

In certain embodiments, the targeting ribonucleic acid is circular.

In certain embodiments, the targeting ribonucleic acid effects the base alteration via binding to an endogenous adenosine deaminase domain.

In certain embodiments, the adenosine deaminase is selected from the group consisting of adenosine deaminase acting on RNA 1 (ADAR1), ADAR2, and ADAR3.

In certain embodiments, the editing agent further comprises: a base editor comprising a polynucleotide programmable RNA binding domain and an adenosine deaminase domain or a cytidine deaminase domain, or a nucleic acid encoding the based editor, wherein the polynucleotide programmable RNA binding domain in conjunction with the targeting ribonucleic acid effects the base alteration.

In certain embodiments, the polynucleotide programmable RNA binding domain comprises a nuclease inactive variant of Cas13 or a nickase variant of Cas13.

In certain embodiments, the Cas13 is Cas13a or Cas13b.

In certain embodiments, (i) the cytidine deaminase domain is selected from the group consisting of the apolipoprotein B mRNA-editing enzyme, catalytic polypeptide-like (APOBEC) family of deaminases, such as APOBEC1, APOBEC2, APOBEC3A, APOBEC3B, APOBEC3C, APOBEC3D/E, APOBEC3F, APOBEC3G, APOBEC3H, or APOBEC4; activation-induced cytidine deaminase (AID), such as activation induced cytidine deaminase (AICDA); cytosine deaminase 1 (CDA1) or CDA2; or cytosine deaminase acting on tRNA (CDAT), and (ii) the adenosine deaminase is selected from the group consisting of adenosine deaminase acting on RNA 1 (ADAR1), ADAR2, ADAR3; adenosine deaminase acting on tRNA 1 (ADAT1), ADAT2, ADAT3; and naturally occurring or engineered tRNA-specific adenosine deaminase (TadA).

In certain embodiments, the targeting ribonucleic acid is a guide RNA or a trigger RNA.

In certain embodiments, the base editor or the targeting ribonucleic acid is encoded by one or more nucleic acid molecules administered to the subject, preferably the base editor is encoded by one or more RNA molecules, such as one or more mRNA molecules, administered to the subject.

In certain embodiments, the targeting ribonucleic acid and/or the one or more nucleic acid molecules, such as the one or more mRNA molecules, are administered to the subject with a lipid nanoparticle (LNP), a peptide cage, or a polymer nanoparticle.

In certain embodiments, the base alteration results in a conversion of an amino acid codon to a premature stop codon, preferably upstream of a splice junction, to thereby down-regulate the expression of the gene.

In certain embodiments, the base alteration results in the conversion of a CGA, CAG, or TGG codon to a premature TGA, TAG, or TAA stop codon, respectively, and the base editor comprises the cytidine deaminase domain, preferably, the CAG codon is located in proximity to the 5′-end of the gene.

In certain embodiments, wherein the base alteration results in a conversion of a premature stop codon to an amino acid codon to thereby up-regulate the expression of the gene.

In certain embodiments, the base alteration results in the conversion of a premature UAG, UAA, or UGA stop codon to a CAG, CAA, or CGA, respectively, and the base editor comprises the adenosine deaminase domain, preferably, the premature UAG stop codon is located in proximity to the 5′-end of the gene.

In certain embodiments, varying amounts of the editing agent, such as varying amounts of the targeting ribonucleic acid are administered to the subject to obtain varying expression levels of the gene.

In certain embodiments, the method further comprises administering to the subject the episomal vector comprising the gene.

In certain embodiments, the episomal vector is a nonviral vector, such as a plasmid, or a viral vector, such as an adeno-associated virus (AAV) vector or an adenovirus vector.

In certain embodiments, the episomal vector is an AAV vector.

In certain embodiments, the subject is a human, such as a human subject suffering from a disease selected from the group consisting of Hereditary angioedema, Pompe disease, hemophilia A, hemophilia B, Fabry disease, Huntington disease, Parkinson's disease, Alzheimer's disease, a synucleinopathy, epilepsy, neuropathic pain, wet macular degeneration, Usher 1F, Usher 1B, glaucoma, Leber congenital amaurosis, and Stargardt disease.

### BRIEF DESCRIPTION OF THE DRAWINGS

FIG. 1A depicts ADAR-based mRNA editing of a STOP codon in a target mRNA to a functional amino acid codon. A single nucleotide change introduces a STOP codon at tyrosine 118 position (W118) of FIX40, and a targeting ribonucleic acid (“trigger RNA”) targets ADAR to edit the TAG (STOP) back to TGG (tyrosine).

FIG. 1B shows FIX40 protein levels measured by WES™ automated capillary-based immunoassay analysis (ProteinSimple, Bio-Techne). Trigger RNA (CadRNA or CadRNAis) was added at two different concentrations (250 or 100 ng). cadRNA is a perfect match to the target mRNA while cadRNAis has mismatches at interspersed loops surrounding the edited position.

FIG. 1C shows FIX40 protein levels in the same samples from FIG. 1B, measured by ELISA.

FIG. 2 shows total mRNA levels for all constructs obtained by quantitative polymerase chain reaction (qPCR) with a primer probe set specific to the FIX sequence.

### DETAILED DESCRIPTION OF THE INVENTION

Various publications, articles and patents are cited or described in the background and throughout the specification; each of these references is herein incorporated by reference in its entirety. Discussion of documents, acts, materials, devices, articles or the like which has been included in the present specification is for the purpose of providing context for the invention. Such discussion is not an admission that any or all of these matters form part of the prior art with respect to any inventions disclosed or claimed.

Unless defined otherwise, all technical and scientific terms used herein have the same meaning commonly understood to one of ordinary skill in the art to which this invention pertains. Otherwise, certain terms cited herein have the meanings as set in the specification.

It must be noted that as used herein and in the appended claims, the singular forms “a,” “an,” and “the” include plural reference unless the context clearly dictates otherwise.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integer or step. When used herein the term “comprising” can be substituted with the term “containing” or “including” or sometimes when used herein with the term “having”.

When used herein “consisting of” excludes any element, step, or ingredient not specified in the claim element, where such element, step or ingredient is related to the claimed invention. When used herein, “consisting essentially of” does not exclude materials or steps that do not materially affect the basic and novel characteristics of the claim. Any of the aforementioned terms of “comprising”, “containing”, “including”, and “having”, whenever used herein in the context of an aspect or embodiment of the invention can be replaced with the term “consisting of” or “consisting essentially of” to vary scopes of the disclosure.

As used herein, the conjunctive term “and/or” between multiple recited elements is understood as encompassing both individual and combined options. For instance, where two elements are conjoined by “and/or”, a first option refers to the applicability of the first element without the second. A second option refers to the applicability of the second element without the first. A third option refers to the applicability of the first and second elements together. Any one of these options is understood to fall within the meaning, and therefore satisfy the requirement of the term “and/or” as used herein. Concurrent applicability of more than one of the options is also understood to fall within the meaning, and therefore satisfy the requirement of the term “and/or.”

All of the features disclosed herein can be combined in any combination. Each feature disclosed in the specification can be replaced by an alternative feature serving a same, equivalent, or similar purpose.

The term “about” as used herein refers to a value within 10% of the underlying parameter (i.e., plus or minus 10%). For example, “about 1:10” means 1.1:10.1 or 0.9:9.9, and about 5 hours means 4.5 hours or 5.5 hours, etc. The term “about” at the beginning of a string of values modifies each of the values by 10%.

All numerical values or numerical ranges include integers within such ranges and fractions of the values or the integers within ranges unless the context clearly indicates otherwise. Thus, to illustrate, reference to reduction of 95% or more includes 95%, 96%, 97%, 98%, 99%, 100% etc., as well as 95.1%, 95.2%, 95.3%, 95.4%, 95.5%, etc., 96.1%, 96.2%, 96.3%, 96.4%, 96.5%, etc., and so forth. Thus, to also illustrate, reference to a numerical range, such as “1-4” includes 2, 3, as well as 1.1, 1.2, 1.3, 1.4, etc., and so forth. For example, “1 to 4 weeks” includes 7, 8, 9, 10, 11, 12, 13, 14, 15, 16, 17, 18, 19, 20, 21, 22, 23, 24, 25, 26, 27, or 28 days.

Further, reference to a numerical range, such as “0.01 to 10” includes 0.011, 0.012, 0.013, etc., as well as 9.5, 9.6, 9.7, 9.8, 9.9, etc., and so forth. For example, a dosage of about “0.01 mg/kg to about 10 mg/kg” body weight of a subject includes 0.011 mg/kg, 0.012 mg/kg, 0.013 mg/kg, 0.014 mg/kg, 0.015 mg/kg etc., as well as 9.5 mg/kg, 9.6 mg/kg, 9.7 mg/kg, 9.8 mg/kg, 9.9 mg/kg etc., and so forth.

Reference to an integer with more (greater) or less than includes any number greater or less than the reference number, respectively. Thus, for example, reference to more than 2 includes 3, 4, 5, 6, 7, 8, 9, 10, 11, 12, 13, 14, 15, etc., and so forth. For example, administration of a non-viral vector and/or immune cell modulator “two or more” times includes 3, 4, 5, 6, 7, 8, 9, 10, 11, 12, 13, 14, 15, or more times.

Further, reference to a numerical range, such as “1 to 90” includes 1.1, 1.2, 1.3, 1.4, 1.5, etc., as well as 81, 82, 83, 84, 85, etc., and so forth. For example, “between about 1 minute to about 90 days” includes 1.1 minutes, 1.2 minutes, 1.3 minutes, 1.4 minutes, 1.5 minutes, etc., as well as one day, 2 days, 3 days, 4 days, 5 days . . . 81 days, 82 days, 83 days, 84 days, 85 days, etc., and so forth.

In an attempt to help the reader of the application, the description has been separated into various paragraphs or sections, or is directed to certain embodiments of the invention. These separations should not be considered as disconnecting the substance of a paragraph or section or embodiments from the substance of another paragraph or section or embodiments. To the contrary, one skilled in the art will understand that the description has broad application and encompasses all the combinations of the various sections, paragraphs and sentences that can be contemplated. The discussion of any embodiment is meant only to be exemplary and is not intended to suggest that the scope of the disclosure, including the claims, is limited to these examples.

Provided herein are methods of regulating expression of a gene located on an episomal vector in a subject in need thereof, comprising administering to the subject one or more gene editing agents that modify a region of the gene to thereby regulate the expression of the gene. Provided herein are also methods of regulating expression of a gene located on an episomal vector in a subject in need thereof, comprising administering to the subject an editing agent that effects an alteration in a region of an mRNA transcript of the gene to thereby regulate the expression of the gene.

The methods of the present invention regulate the expression of a transgene provided by a gene therapy. The regulation of the expression of the gene therapy transgene can be effected in a wide variety of ways including, without limitation, alteration of the level, potency, activity, tertiary structure or folding of the protein product or regulatory RNA product of the gene therapy transgene.

For example, coding mutations may target catalytic serine, lysine, arginine, or histidine residues in the active site of enzymes, modification of lysine acetylation or ubiquitination sites (lysine to glutamate or alanine substitutions), substitution of serine or threonine phosphorylation sites (threonine or serine to alanine), asparagine-linked (“N-linked”) glycosylation sites (asparagine to aspartate), or modification of lipid binding domains by replacement of histidine, lysine, or arginine residues critical for binding lipid head groups in phosphoinositides (mutation to non-polar or negatively charged residues).

In certain embodiments, the alteration in a region of an mRNA transcript of the gene alters the stability of the mRNA transcript, the initiation or level of the translation of the mRNA transcript, the stability and/or activity of the translated protein.

The term “vector” or “expression vector” as used herein, refers to a vector, and in particular, an episomal vector. A vector is generally a plasmid that is used to introduce and express a specific gene into a target cell. The expression vector allows production of large amounts of stable mRNA. Once the expression vector is inside the cell, the protein that is encoded by the gene is produced by the cellular transcription and translation machinery. The plasmid is engineered such that it contains a highly active promoter which causes the production of large amounts of mRNA. An “episomal vector” is capable of self-replicating autonomously within the host cells. In certain embodiments, the episomal vector is a nonviral vector, such as a plasmid, or a viral vector, such as an adeno-associated viral (AAV) vector, a lentivirus vector, or an adenovirus vector.

In certain embodiments, the region of the gene comprises one or more of a promoter, an enhancer, a silencer or an insulator, or the region of the gene or the region of the mRNA transcript comprises a premature termination codon convertible to an amino acid codon via the base alteration, or an amino acid codon convertible to a premature termination codon via the base alteration.

The term “alteration” as used herein refers to a change in a polynucleotide or polypeptide sequence or a change in expression levels, such as a 10% change, a 25% change, a 40% change, a 50% change, or greater.

The term “gene,” as used herein, refers to a set of segments of nucleic acid that contains the information necessary to produce a functional RNA product in a controlled manner through a transcription process. This RNA can then be used directly (such as tRNA, rRNA, snRNAs and other non-coding RNAs (e.g., the SRP RNAs), anti-sense RNA, or micro-RNA) or to direct the synthesis of proteins. When the phrase “a gene encoding a protein” or “a protein is encoded by a gene” is used, it means that the gene is transcribed into an mRNA which then is translated into a protein, including post- and peri-translation which occur in the mammalian cells.

The terms “nucleic acid” and “polynucleotide” are used interchangeably herein to refer to all forms of nucleic acid, oligonucleotides, including deoxyribonucleic acid (DNA) and ribonucleic acid (RNA). In discussing nucleic acids, a sequence or structure of a particular polynucleotide can be described herein according to the convention of providing the sequence in the 5′ to 3′ direction.

In certain embodiments, nucleic acids include genomic DNA, cDNA, antisense DNA/RNA, plasmid DNA, linear DNA, (poly- and oligo-nucleotide), chromosomal DNA, spliced or unspliced mRNA, rRNA, tRNA inhibitory DNA or RNA (RNAi, e.g., small or short hairpin (sh)RNA, microRNA (miRNA), small or short interfering (si)RNA, trans-splicing RNA, or antisense RNA), locked nucleic acid analogue (LNA), oligonucleotide DNA (ODN) single and double stranded, immunostimulating sequence (ISS), riboswitches and ribozymes.

The term “mRNA” or sometimes refer by “mRNA transcripts” as used herein, include, but not limited to pre-mRNA transcript(s), transcript processing intermediates, mature mRNA(s) ready for translation and transcripts of the gene or genes, or nucleic acids derived from the mRNA transcript(s).

The term “nucleobase,” “nitrogenous base,” or “base,” used interchangeably herein, refers to a nitrogen-containing biological compound that forms a nucleoside, which in turn is a component of a nucleotide. The ability of nucleobases to form base pairs and to stack one upon another leads directly to long-chain helical structures such as ribonucleic acid (RNA) and deoxyribonucleic acid (DNA). Five nucleobases—adenine (A), cytosine (C), guanine (G), thymine (T), and uracil (U)— are called primary or canonical. Adenine and guanine are derived from purine, and cytosine, uracil, and thymine are derived from pyrimidine. DNA and RNA can also contain other (non-primary) bases that are modified. Non-limiting exemplary modified nucleobases can include hypoxanthine, xanthine, 7-methylguanine, 5,6-dihydrouracil, 5-methylcytosine (m5C), and 5-hydromethylcytosine. Hypoxanthine and xanthine can be created through mutagen presence, both of them through deamination(replacement of the amine group with a carbonyl group). Hypoxanthine can be modified from adenine. Xanthine can be modified from guanine. Uracil can result from deamination of cytosine. A “nucleoside” consists of a nucleobase and a five-carbon sugar (either ribose or deoxyribose). Examples of a nucleoside include adenosine, guanosine, uridine, cytidine, 5-methyluridine (m5U), deoxyadenosine, deoxyguanosine, thymidine, deoxyuridine, and deoxycytidine. Examples of a nucleoside with a modified nucleobase includes inosine (I), xanthosine (X), 7-methylguanosine (m7G), dihydrouridine (D), 5-methylcytidine (m5C), and pseudouridine (Y). A “nucleotide” consists of a nucleobase, a five-carbon sugar (either ribose or deoxyribose), and at least one phosphate group.

The terms “identity,” “homology,” and grammatical variations thereof, mean that two or more referenced entities are the same, when they are “aligned” sequences. Thus, by way of example, when two nucleic acids are identical, they have the same sequence, at least within the referenced region or portion. The identity can be over a defined area (region or domain) of the sequence.

An “area” or “region” of identity refers to a portion of two or more referenced entities that are the same. Thus, where two protein or nucleic acid sequences are identical over one or more sequence areas or regions they share identity within that region. An “aligned” sequence refers to multiple protein (amino acid) or nucleic acid sequences, often containing corrections for missing or additional bases or amino acids (gaps) as compared to a reference sequence.

The identity can extend over the entire length or a portion of the sequence. In certain embodiments, the length of the sequence sharing the percent identity is 2, 3, 4, 5 or more contiguous amino acids or nucleic acids, e.g., 6, 7, 8, 9, 10, 11, 12, 13, 14, 15, 16, 17, 18, 19, 20, etc. contiguous nucleic acids or amino acids. In certain embodiments, the length of the sequence sharing identity is 21 or more contiguous amino acids or nucleic acids, e.g., 21, 22, 23, 24, 25, 26, 27, 28, 29, 30, 31, 32, 33, 34, 35, 36, 37, 38, 39, 40, etc. contiguous amino acids or nucleic acids. In further embodiments, the length of the sequence sharing identity is 41 or more contiguous amino acids or nucleic acids, e.g., 42, 43, 44, 45, 45, 47, 48, 49, 50, etc., contiguous amino acids or nucleic acids. In yet further embodiments, the length of the sequence sharing identity is 50 or more contiguous amino acids or nucleic acids, e.g., 50-55, 55-60, 60-65, 65-70, 70-75, 75-80, 80-85, 85-90, 90-95, 95-100, 100-150, 150-200, 200-250, 250-300, 300-500, 500-1,000, etc. contiguous amino acids or nucleic acids.

As used herein, the term “promoter” refers to a sequence capable of controlling the expression of a coding sequence or functional RNA. In general, nucleic acid molecules are located 3′ of a promoter sequence. In certain embodiments, a promoter sequence consists of proximal and more distal upstream elements and can comprise an enhancer element.

An “enhancer” as used herein can refer to a sequence that is located adjacent to the heterologous nucleic acid. Enhancer elements are typically located upstream of a promoter element but also function and can be located downstream of or within a sequence. Hence, an enhancer element can be located 10-50 base pairs, 50-100 base pairs, 100-200 base pairs, or 200-300 base pairs, or more base pairs upstream or downstream of a heterologous nucleic acid sequence. Enhancer elements typically increase expression of an operably linked nucleic acid afforded by a promoter element.

As used herein, the term “silencer” refers to a sequence-specific element that induces a negative effect on the transcription of a gene.

As used herein, the term “insulator” or “insulating sequence” refer to a type of cis-regulatory element known as a long-range regulatory element. Insulating sequences are segments of DNA that block interactions or interference of neighboring gene sequences. For example, insulators can reduce the transcriptional read through from a promoter of a neighboring gene or spurious promoters in adjacent nucleotide sequences. Or they block the interaction of an enhancer on one side of the insulating sequence with a promoter of a neighboring gene on the other side of the insulating sequence. The defining characteristic of an insulating sequence within the meaning of the present invention is its ability to insulate or protect a defined transcription unit which is operably linked to a regulatory element from the influence of an upstream or downstream interfering genetic element. For this purpose, the insulating sequence is placed between the (potential) interfering genetic sequence and the regulatory sequence of the transcription unit to be insulated.

As used herein, the term “stop codon” (also referred to as termination codon) is a nucleotide triplet within messenger RNA that signals termination of translation, as opposed to most codons in messenger RNA that correspond to the addition of an amino acid residue to a growing polypeptide chain. Thus, the term “premature termination codon” or “premature stop codon” refers to the occurrence of a stop codon instead of a codon corresponding to an amino acid residue. The premature stop codon may be located anywhere upstream to the normal stop codon which is regularly located at the end of the coding nucleic acid sequence of a particular gene. The premature termination codon may be any one of the known stop codons, including TAG (transcribed as UAG), TAA (transcribed as UAA) and TGA (transcribed as UGA).

### Gene Editing

In certain embodiments, a method of the invention comprises administering to the subject one or more gene editing agents that modify a region of the gene to thereby regulate the expression of the gene. Gene editing agents include agents that can target the genome of a cell to modify expression of a gene. The term “gene editing agent” as used herein encompasses gene editing agents that cleave the targeted DNA to induce mutation (e.g., via homologous directed repair or non-homologous end-joining).

The term “mutation,” as used herein, refers to a substitution of a residue within a sequence, e.g., a nucleic acid or amino acid sequence, with another residue, or a deletion or insertion of one or more residues within a sequence. Mutations are typically described herein by identifying the original residue followed by the position of the residue within the sequence and by the identity of the newly substituted residue. Various methods for making the amino acid substitutions (mutations) provided herein are well known in the art, and are provided by, for example, Green and Sambrook, Molecular Cloning: A Laboratory Manual (4th ed., Cold Spring Harbor Laboratory Press, Cold Spring Harbor, N.Y. (2012)).

In certain embodiments, the nucleic acid binding protein is a (modified) transcription activator-like effector nuclease (TALEN) system. Transcription activator-like effectors (TALEs) can be engineered to bind practically any desired DNA sequence. Exemplary methods of genome editing using the TALEN system can be found for example in Cermak T. Doyle E L. Christian M. Wang L. Zhang Y. Schmidt C, et al. Efficient design and assembly of custom TALEN and other TAL effector-based constructs for DNA targeting. Nucleic Acids Res. 2011; 39:e82; Zhang F. Cong L. Lodato S. Kosuri S. Church G M. Arlotta P Efficient construction of sequence-specific TAL effectors for modulating mammalian transcription. Nat Biotechnol. 2011; 29: 149-153 and U.S. Pat. Nos. 8,450,471, 8,440,431 and 8,440,432, all of which are specifically incorporated by reference. By means of further guidance, and without limitation, naturally occurring TALEs or “wild type TALEs” are nucleic acid binding proteins secreted by numerous species of proteobacteria. TALE polypeptides contain a nucleic acid binding domain composed of tandem repeats of highly conserved monomer polypeptides that are predominantly 33, 34 or 35 amino acids in length and that differ from each other mainly in amino acid positions 12 and 13. In advantageous embodiments the nucleic acid is DNA. As used herein, the term “polypeptide monomers”, or “TALE monomers” will be used to refer to the highly conserved repetitive polypeptide sequences within the TALE nucleic acid binding domain and the term “repeat variable di-residues” or “RVD” will be used to refer to the highly variable amino acids at positions 12 and 13 of the polypeptide monomers. As provided throughout the disclosure, the amino acid residues of the RVD are depicted using the IUPAC single letter code for amino acids. A general representation of a TALE monomer which is comprised within the DNA binding domain is X₁₋₁₁-(X₁₂X₁₃)-X₁₄₋₃₃ or 34 or 35, where the subscript indicates the amino acid position and X represents any amino acid. X₁₂X₁₃ indicate the RVDs. In some polypeptide monomers, the variable amino acid at position 13 is missing or absent and in such polypeptide monomers, the RVD consists of a single amino acid. In such cases the RVD may be alternatively represented as X*, where X represents X12 and (*) indicates that X13 is absent. The DNA binding domain comprises several repeats of TALE monomers and this may be represented as (X₁₋₁₁-(X₁₂X₁₃)-X₁₄₋₃₃ or ₃₄ or ₃₅)z, where in an advantageous embodiment, z is at least 5 to 40. In a further advantageous embodiment, z is at least 10 to 26. The TALE monomers have a nucleotide binding affinity that is determined by the identity of the amino acids in its RVD. For example, polypeptide monomers with an RVD of NI preferentially bind to adenine (A), polypeptide monomers with an RVD of NG preferentially bind to thymine (T), polypeptide monomers with an RVD of HD preferentially bind to cytosine (C) and polypeptide monomers with an RVD of NN preferentially bind to both adenine (A) and guanine (G). In yet another embodiment of the invention, polypeptide monomers with an RVD of IG preferentially bind to T. Thus, the number and order of the polypeptide monomer repeats in the nucleic acid binding domain of a TALE determines its nucleic acid target specificity. In still further embodiments of the invention, polypeptide monomers with an RVD of NS recognize all four base pairs and may bind to A, T, G or C. The structure and function of TALEs is further described in, for example, Moscou et al., Science 326: 1501 (2009); Boch et al., Science 326: 1509-1512 (2009); and Zhang et al., Nature Biotechnology 29: 149-153 (2011), each of which is incorporated by reference in its entirety. In certain embodiments, targeting is effected by a polynucleic acid binding TALEN fragment. In certain embodiments, the targeting domain comprises or consists of a catalytically inactive TALEN or nucleic acid binding fragment thereof.

In certain embodiments, the targeting domain comprises or consists of a (modified) zinc-finger nuclease (ZFN) system. The ZFN system uses artificial restriction enzymes generated by fusing a zinc finger DNA-binding domain to a DNA-cleavage domain that can be engineered to target desired DNA sequences. Exemplary methods of genome editing using ZFNs can be found for example in U.S. Pat. Nos. 6,534,261, 6,607,882, 6,746,838, 6,794, 136, 6,824,978, 6,866,997, 6,933, 113, 6,979,539, 7,013,219, 7,030,215, 7,220,719, 7,241,573, 7,241,574, 7,585,849, 7,595,376, 6,903, 185, and 6,479,626, all of which are specifically incorporated by reference. By means of further guidance, and without limitation, artificial zinc-finger (ZF) technology involves arrays of ZF modules to target new DNA-binding sites in the genome. Each finger module in a ZF array targets three DNA bases. A customized array of individual zinc finger domains is assembled into a ZF protein (ZFP). ZFPs can comprise a functional domain. The first synthetic zinc finger nucleases (ZFNs) were developed by fusing a ZF protein to the catalytic domain of the Type IIS restriction enzyme Fokl. (Kim, Y. G. et al., 1994, Chimeric restriction endonuclease, Proc. Natl. Acad. Sci. U.S.A. 91, 883-887; Kim, Y. G. et al., 1996, Hybrid restriction enzymes: zinc finger fusions to Fok I cleavage domain. Proc. Natl. Acad. Sci. U.S.A. 93, 1156-1160). Increased cleavage specificity can be attained with decreased off target activity by use of paired ZFN heterodimers, each targeting different nucleotide sequences separated by a short spacer. (Doyon, Y. et al., 2011, Enhancing zinc-finger-nuclease activity with improved obligate heterodimeric architectures. Nat. Methods 8, 74-79). ZFPs can also be designed as transcription activators and repressors and have been used to target many genes in a wide variety of organisms. In certain embodiments, the targeting domain comprises or consists of a nucleic acid binding zinc finger nuclease or a nucleic acid binding fragment thereof. In certain embodiments, the nucleic acid binding (fragment of) a zinc finger nuclease is catalytically inactive.

In certain embodiments, the targeting domain comprises a (modified) meganuclease, which are endodeoxyribonucleases characterized by a large recognition site (double-stranded DNA sequences of 12 to 40 base pairs). Exemplary method for using meganucleases can be found in U.S. Pat. Nos: 8,163,514; 8,133,697; 8,021,867; 8,119,361; 8,119,381; 8,124,369; and 8,129,134, which are specifically incorporated by reference. In certain embodiments, targeting is effected by a polynucleic acid binding meganuclease fragment. In certain embodiments, targeting is effected by a polynucleic acid binding catalytically inactive meganuclease (fragment). Accordingly in particular embodiments, the targeting domain comprises or consists of a nucleic acid binding meganuclease or a nucleic acid binding fragment thereof.

In certain embodiments, the targeting domain comprises a (modified) CRISPR/Cas complex or system. General information on CRISPR/Cas Systems, components thereof, and delivery of such components, including methods, materials, delivery vehicles, vectors, particles, and making and using thereof, including as to amounts and formulations, as well as CRISPR/Cas-expressing eukaryotic cells, CRISPR/Cas expressing eukaryotes, such as a mouse, is described herein elsewhere. In certain embodiments, targeting is effected by an oligonucleic acid binding CRISPR protein fragment and/or a gRNA. In certain embodiments, targeting is effected by a nucleic acid binding catalytically inactive CRISPR protein (fragment). Accordingly in particular embodiments, the targeting domain comprises oligonucleic acid binding CRISPR protein or an oligonucleic acid binding fragment of a CRISPR protein and/or a gRNA.

In certain embodiments, the one or more gene editing agents comprise a guide RNA that is complementary to the region of the gene and a Cas protein or a derivative of the Cas protein.

The terms “guide RNA” or “gRNA” refers to a polynucleotide which can be specific for a target sequence and can form a complex with a polynucleotide programmable nucleotide binding domain protein (e.g., Cas9 or Cas13). In an embodiment, the guide polynucleotide is a guide RNA (gRNA). gRNAs can exist as a complex of two or more RNAs, or as a single RNA molecule. gRNAs that exist as a single RNA molecule may be referred to as single-guide RNAs (sgRNAs), though “gRNA” is used interchangeably to refer to guide RNAs that exist as either single molecules or as a complex of two or more molecules. Typically, gRNAs that exist as single RNA species comprise two domains: (1) a domain that shares homology to a target nucleic acid (e.g., and directs binding of a Cas9 complex to the target); and (2) a domain that binds a Cas9 protein. In some embodiments, domain (2) corresponds to a sequence known as a tracrRNA and comprises a stem-loop structure. For example, in some embodiments, domain (2) is identical or homologous to a tracrRNA as provided in Jinek et al., Science 337:816-821(2012), the entire contents of which is incorporated herein by reference.

As used herein, the term “Cas” generally refers to a (modified) effector protein of the CRISPR/Cas system or complex. Non-limiting examples of Cas enzymes include Cas1, Cas1B, Cas2, Cas3, Cas4, Cas5, Cas5d, Cas5t, Cas5h, Cas5a, Cas6, Cas7, Cas8, Cas8a, Cas8b, Cas8c, Cas9 (also known as Csn1 or Csx12), Cas10, Cas10d, Cas12a/Cpf1, Cas12b/C2c1, Cas12c/C2c3, Cas12d/CasY, Cas12e/CasX, Cas12g, Cas12h, Cas12i, Cas13a/C2c2, Cas13b, Cas13c, Cas13d, Csy1, Csy2, Csy3, Csy4, Cse1, Cse2, Cse3, Cse4, Cse5e, Csc1, Csc2, Csa5, Csn1, Csn2, Csm1, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx1S, Csx11, Csf1, Csf2, CsO, Csf4, Csd1, Csd2, Cst1, Cst2, Csh1, Csh2, Csa1, Csa2, Csa3, Csa4, Csa5, Type II Cas effector proteins, Type V Cas effector proteins, Type VI Cas effector proteins, CARF, DinG, homologues thereof, or modified or engineered versions thereof. Other nucleic acid programmable DNA binding proteins are also within the scope of this disclosure, although they may not be specifically listed in this disclosure. See, e.g., Makarova et al.

The term “Cas” may be used herein interchangeably with the terms “CRISPR” protein, “CRISPR/Cas protein”, “CRISPR effector”, “CRISPR/Cas effector”, “CRISPR enzyme”, “CRISPR/Cas enzyme” and the like, unless otherwise apparent, such as by specific and exclusive reference to Cas9. It is to be understood that the term “CRISPR protein” may be used interchangeably with “CRISPR enzyme”, irrespective of whether the CRISPR protein has altered, such as increased or decreased (or no) enzymatic activity, compared to the wild type CRISPR protein. Likewise, as used herein, in certain embodiments, where appropriate and which will be apparent to the skilled person, the term “nuclease” may refer to a modified nuclease wherein catalytic activity has been altered, such as having increased or decreased nuclease activity, or no nuclease activity at all, as well as nickase activity, as well as otherwise modified nuclease as defined herein elsewhere, unless otherwise apparent, such as by specific and exclusive reference to unmodified nuclease.

In some embodiments, the Cas protein is Cas9, such as Staphylococcus aureus Cas9 (SaCas9), Streptococcus thermophilus 1 Cas9 (St1Cas9), a modified Streptococcus pyogenes Cas9 (SpCas9); a CpF1; a CasX; a CasY; a C2c1; a C2c2; a C2c3; or a variant thereof, or Cas13a, Cas13b, Cas13c, or Cas13d. In some embodiments, the Cas protein is a DNA-targeting CRISPR effector protein. In some embodiments, the Cas protein is a Type-II CRISPR effector protein such as Cas9. In some embodiments, the CRISPR effector protein is a Type-V CRISPR effector protein such as Cpf1 or C2cl. In some embodiments, the Cas protein is a RNA-targeting CRISPR effector protein. In some embodiments, the CRISPR effector protein is a Type-VI CRISPR effector protein such as Cas13a, Cas13b, Cas13c, or Cas13d.

In certain embodiments, the one or more gene editing agents further comprise a donor nucleic acid having at least one nucleotide change relative to the region of the gene and capable of integrating into the region of the gene to modify the region.

In certain embodiments, the one or more gene editing agents are encoded by one or more nucleic acid molecules administered to the subject, preferably the one or more gene editing agents are encoded by an RNA molecule, particularly an mRNA molecule, administered to the subject.

As used herein the term “donor DNA” or “donor nucleic acid” refers to nucleic acid that is designed to be introduced into a locus by homologous recombination. Donor nucleic acid will have at least one region of sequence homology to the locus. In many instances, donor nucleic acid will have two regions of sequence homology to the locus. These regions of homology may be at one of both termini or may be internal to the donor nucleic acid. In many instances, and “insert” region with nucleic acid that one desires to be introduced into nucleic acid molecules present in a cell will be located between two regions of homology.

### Base Editing

Also provided are methods of regulating expression of a gene located on an episomal vector in a subject in need thereof, comprising administering to the subject a base editor system that effects a base alteration in a region of the gene or a region of an mRNA transcript of the gene to thereby regulate the expression of the gene.

Also provided is a method of regulating expression of a gene located on an episomal vector in a subject in need thereof, comprising administering to the subject an editing agent that effects an alteration in a region of an mRNA transcript of the gene to thereby regulate the expression of the gene.

In certain embodiments, the editing agent effects a base alteration in the region of the mRNA transcript of the gene.

The term “base editor system” refers to a system for editing a nucleobase of a target nucleotide sequence. In certain embodiments, the base editor system comprises: a ribonucleic acid complementary to the region of the gene; and a base editor comprising a polynucleotide programmable DNA binding domain and an adenosine deaminase domain or a cytidine deaminase domain, wherein the polynucleotide programmable DNA binding domain in conjunction with the ribonucleic acid binds to the region of the gene to effect the base alteration.

The term “base editor (BE),” or “nucleobase editor (NBE)” refers to an agent comprising a polypeptide that is capable of making a modification to a base (e.g., A, T, C, G, or U) within a nucleic acid sequence (e.g., DNA or RNA). In some embodiments, the base editor is capable of deaminating a base within a nucleic acid. In some embodiments, the base editor is capable of deaminating a base within a DNA molecule. By “base editing activity” is meant acting to chemically alter a base within a polynucleotide. In one embodiment, a first base is converted to a second base. In one embodiment, the base editing activity is cytidine deaminase activity, e.g., converting target C*G to T*A. In another embodiment, the base editing activity is adenosine deaminase activity, e.g., converting A*T to G*C.

The term “nucleic acid programmable DNA binding protein” or “napDNAbp” may be used interchangeably with “polynucleotide programmable nucleotide binding domain” to refer to a protein that associates with a nucleic acid (e.g., DNA or RNA), such as a guide nucleic acid or guide polynucleotide (e.g., gRNA), that guides the napDNAbp to a specific nucleic acid sequence. Non-limiting examples of a polynucleotide programmable nucleotide binding domain which can be incorporated into a base editor include a CRISPR protein-derived domain, a restriction nuclease, a meganuclease, TAL nuclease (TALEN), and a zinc finger nuclease (ZFN). In some embodiments, the polynucleotide programmable nucleotide binding domain is a polynucleotide programmable DNA binding domain. In some embodiments, the polynucleotide programmable nucleotide binding domain is a polynucleotide programmable RNA binding domain. In some embodiments, the polynucleotide programmable nucleotide binding domain is a Cas9 protein. A Cas9 protein can associate with a guide RNA that guides the Cas9 protein to a specific DNA sequence that is complementary to the guide RNA.

In some embodiments, the polynucleotide programmable DNA binding domain comprises a nuclease inactive variant of a Cas protein or a nickase variant of a Cas protein. In certain embodiments, the Cas protein is Cas9, such as Staphylococcus aureus Cas9 (SaCas9), Streptococcus thermophilus 1 Cas9 (St1Cas9), a modified Streptococcus pyogenes Cas9 (SpCas9); a CpF1; a CasX; a CasY; a C2c1; a C2c2; a C2c3; or a variant thereof.

In certain embodiments, the base editor further comprises a base repair inhibitor. In some embodiments, the base repair inhibitor is uracil glycosylase inhibitor (UGI). UGI refers to a protein that is capable of inhibiting a uracil-DNA glycosylase base-excision repair enzyme. In some embodiments, a UGI domain comprises a wild-type UGI or a fragment of a wild-type UGI. In some embodiments, the UGI proteins provided herein include fragments of UGI and proteins homologous to a UGI or a UGI fragment. In some embodiments, the base repair inhibitor is an inhibitor of inosine base excision repair. In some embodiments, the base repair inhibitor is a “catalytically inactive inosine specific nuclease” or “dead inosine specific nuclease. Without wishing to be bound by any particular theory, catalytically inactive inosine glycosylases (e.g., alkyl adenine glycosylase (AAG)) can bind inosine, but cannot create an abasic site or remove the inosine, thereby sterically blocking the newly formed inosine moiety from DNA damage/repair mechanisms. In some embodiments, the catalytically inactive inosine specific nuclease can be capable of binding an inosine in a nucleic acid but does not cleave the nucleic acid. Non-limiting exemplary catalytically inactive inosine specific nucleases include catalytically inactive alkyl adenosine glycosylase (AAG nuclease), for example, from a human, and catalytically inactive endonuclease V (EndoV nuclease), for example, from E. coli. In some embodiments, the catalytically inactive AAG nuclease comprises an E125Q mutation or a corresponding mutation in another AAG nuclease. In some embodiments, base editor further comprises a uracil binding protein, such as a uracil glycosylase inhibitor (UGI) domain that inhibits a uracil-DNA glycosylase.

As used herein, the term “deaminase” or “deaminase domain” or “deaminase moiety” refers to a protein or enzyme that catalyzes a deamination reaction. In some embodiments, the deaminase is an adenosine deaminase, which catalyzes the hydrolytic deamination of adenine or adenosine (e.g., an engineered adenosine deaminase that deaminates adenosine in DNA). In some embodiments, the deaminase or deaminase domain is a cytidine deaminase, catalyzing the hydrolytic deamination of cytidine or deoxycytidine to uridine or deoxyuridine, respectively. In some embodiments, the deaminase or deaminase domain is a cytidine deaminase domain, catalyzing the hydrolytic deamination of cytosine to uracil. In some embodiments, the deaminase or deaminase domain is a naturally-occurring deaminase from an organism, such as a human, chimpanzee, gorilla, monkey, cow, dog, rat, or mouse. In some embodiments, the deaminase or deaminase domain is a variant of a naturally-occurring deaminase from an organism that does not occur in nature. For example, in some embodiments, the deaminase or deaminase domain is at least 50%, at least 55%, at least 60%, at least 65%, at least 70%, at least 75% at least 80%, at least 85%, at least 90%, at least 95%, at least 96%, at least 97%, at least 98%, at least 99%, or at least 99.5% identical to a naturally-occurring deaminase from an organism. The term deaminase also embraces any genetically engineered deaminase that may comprise genetic modifications (e.g., one or more mutations) that results in a variant deaminase having an amino acid sequence comprising one or more changes relative to a wildtype counterpart deaminase. Examples of deaminases are given herein, and the term is not meant to be limiting.

The term “adenosine deaminase” refers to a polypeptide or fragment thereof capable of catalyzing the hydrolytic deamination of adenine or adenosine. In some embodiments, the deaminase or deaminase domain is an adenosine deaminase catalyzing the hydrolytic deamination of adenosine to inosine or deoxy adenosine to deoxyinosine. A base editor comprising an adenosine deaminase can act on any polynucleotide, including DNA, RNA and DNA-RNA hybrids (Zheng et al. Nucleic Acids Res. 2017, 45(6):3369-3377). A base editor comprising an adenosine deaminase domain can be capable of deaminating an A nucleobase of a DNA polynucleotide. In an embodiment, an adenosine deaminase domain of a base editor comprises all or a portion of adenosine deaminase acting on DNA (e.g., an adenosine deaminase 1 (ADA1) or ADA2). In certain embodiments, a base editor comprising an adenosine deaminase can deaminate a target A of a polynucleotide comprising RNA. In an embodiment, an adenosine deaminase incorporated into a base editor comprises all or a portion of adenosine deaminase acting on RNA (ADAR, e.g., ADAR1 or ADAR2). In another embodiment, an adenosine deaminase incorporated into a base editor comprises all or a portion of adenosine deaminase acting on tRNA (ADAT, e.g., ADAT1 ADAT2, or ADAT3, or naturally occurring or engineered tRNA-specific adenosine deaminase (TadA)). In particular embodiments, the TadA is any one of the TadA described in PCT/US2017/045381, which is incorporated herein by reference in its entirety. The following table provides exemplary sequences; other sequences can also be used.

TABLE 1

| Deaminase | Nucleic Acid | Amino Acid |
|---|---|---|
| ADA (ADA1) | NM_000022.3 variant 1 (SEQ ID NO: 4) | NP_000013.2 isoform 1 (SEQ ID NO: 5) |
| ADA2 | NM_001282225.1 (SEQ ID NO: 6) | NP_001269154.1 (SEQ ID NO: 7) |
| ADAR | NM_001111.4 (SEQ ID NO: 8 | NP_001102.2 (SEQ ID NO: 9) |
| ADAR2 | NM_001112.3 variant 1 (SEQ ID NO: 10) | NP_001103.1 isoform 1 (SEQ ID NO: 11) |
| ADAR3 | NM_018702.3 (SEQ ID NO: 12) | NP_061172.1 (SEQ ID NO: 13) |
| ADAT1 | NM_012091.4 variant 1 (SEQ ID NO: 14) | NP_036223.2 isoform 1 (SEQ ID NO: 15) |
| ADAT2 | NM_182503.2 variant 1 (SEQ ID NO: 16 | NP_872309.2 isoform 1 (SEQ ID NO: 17) |
| ADAT3 | NM_138422.3 variant 1 (SEQ ID NO: 18) | NP_612431.2 isoform 1 (SEQ ID NO: 19) |

The term “cytidine deaminase” refers to a polypeptide or fragment thereof capable of catalyzing a deamination reaction that converts an amino group to a carbonyl group. In one embodiment, the cytidine deaminase converts cytosine to uracil or 5-methylcytosine to thymine. In certain embodiments, the cytidine deaminase is selected from the group consisting of the apolipoprotein B mRNA-editing enzyme, catalytic polypeptide-like (APOBEC) family of deaminases, such as APOBEC1, APOBEC2, APOBEC3A, APOBEC3B, APOBEC3C, APOBEC3D/E, APOBEC3F, APOBEC3G, APOBEC3H, or APOBEC4; activation-induced cytidine deaminase (AID), such as activation induced cytidine deaminase (AICDA); cytosine deaminase 1 (CDA1) or CDA2; or cytosine deaminase acting on tRNA (CDAT). The following table provides exemplary sequences; other sequences can also be used.

TABLE 2

| Deaminase | Nucleic Acid | Amino Acid |
|---|---|---|
| hAID/AICDA | NM_020661.3 isoform 1 | NP_065712.1 variant 1 |
| | (SEQ ID NO: 20) | (SEQ ID NO: 21) |
| | NM_020661.3 isoform 2 | NP_065712.1 variant 2 |
| | (SEQ ID NO: 22) | (SEQ ID NO: 23) |
| APOBEC1 | NM_001644.4 isoform a | NP_001635.2 variant 1 |
| | (SEQ ID NO: 24) | (SEQ ID NO: 25) |
| | NM_005889.3 isoform b | NP_005880.2 variant 3 |
| | (SEQ ID NO: 26) | (SEQ ID NO: 27) |
| APOBEC2 | NM_006789.3 (SEQ ID NO: 28) | NP_006780.1 (SEQ ID NO: 29) |
| APOBEC3A | NM_145699.3 isoform a | NP_663745.1 variant 1 |
| | (SEQ ID NO: 30) | (SEQ ID NO: 31) |
| | NM_001270406.1 isoform b | NP_001257335.1 variant 2 |
| | (SEQ ID NO: 32) | (SEQ ID NO: 33) |
| APOBEC3B | NM_004900.4 isoform a | NP_004891.4 variant 1 |
| | (SEQ ID NO: 34) | (SEQ ID NO: 35) |
| | NM_001270411.1 isoform b | NP_001257340.1 variant 2 |
| | (SEQ ID NO: 36) | (SEQ ID NO: 37) |
| APOBEC3C | NM_014508.2 (SEQ ID NO: 38) | NP_055323.2 (SEQ ID NO: 39) |
| APOBEC3D/E | NM_152426.3 (SEQ ID NO: 40) | NP_689639.2 (SEQ ID NO: 41) |
| APOBEC3F | NM_145298.5 isoform a | NP_660341.2 variant 1 |
| | (SEQ ID NO: 42) | (SEQ ID NO: 43) |
| | NM_001006666.1 isoform b | NP_001006667.1 variant 2 |
| | (SEQ ID NO: 44) | (SEQ ID NO: 45) |
| APOBEC3G | NM_021822.3 (isoform a) | NP_068594.1 (variant 1) |
| | (SEQ ID NO: 46) | (SEQ ID NO: 47) |
| APOBEC3H | NM_001166003.2 (SEQ ID NO: 48) | NP_001159475.2 (variant SV-200) |
| | | (SEQ ID NO: 49) |
| APOBEC4 | NM_203454.2 (SEQ ID NO: 50) | NP_982279.1 (SEQ ID NO: 51) |
| CDA1* | NM_127515.4 (SEQ ID NO: 52) | NP_179547.1 (SEQ ID NO: 53) |

*from Saccharomyces cerevisiae S288C

In some embodiments, the base editor is a Cas9 fused to a deaminase (e.g., an adenosine deaminase or cytidine deaminase). In some embodiments, the base editor is a nuclease-inactive Cas9 (dCas9) fused to a deaminase (e.g., an adenosine deaminase or cytidine deaminase).

The term “linker”, as used herein, can refer to a covalent linker (e.g., covalent bond), a non-covalent linker, a chemical group, or a molecule linking two molecules or moieties, e.g., two components of a protein complex or a ribonucleocomplex, or two domains of a fusion protein, such as, for example, a polynucleotide programmable DNA binding domain (e.g., dCas9) and a deaminase domain ((e.g., an adenosine deaminase, a cytidine deaminase, or an adenosine deaminase and a cytidine deaminase; see PCT/US2019/044935, PCT/US2020/016288, each of which is incorporated herein by reference for its entirety). A linker can join different components of, or different portions of components of, a base editor system. For example, in some embodiments, a linker can join a guide polynucleotide binding domain of a polynucleotide programmable nucleotide binding domain and a catalytic domain of a deaminase. In some embodiments, a linker can join a CRISPR polypeptide and a deaminase. In some embodiments, a linker can join a Cas9 and a deaminase. In some embodiments, a linker can join a dCas9 and a deaminase. In some embodiments, a linker can join a nCas9 and a deaminase. In some embodiments, a linker can join a guide polynucleotide and a deaminase. In some embodiments, a linker can join a deaminating component and a polynucleotide programmable nucleotide binding component of a base editor system. In some embodiments, a linker can join a RNA-binding portion of a deaminating component and a polynucleotide programmable nucleotide binding component of a base editor system. In some embodiments, a linker can join a RNA-binding portion of a deaminating component and a RNA-binding portion of a polynucleotide programmable nucleotide binding component of a base editor system. A linker can be positioned between, or flanked by, two groups, molecules, or other moieties and connected to each one via a covalent bond or non-covalent interaction, thus connecting the two. In some embodiments, the linker can be an organic molecule, group, polymer, or chemical moiety. In some embodiments, the linker can be a polynucleotide. In some embodiments, the linker can be a DNA linker. In some embodiments, the linker can be a RNA linker.

A “targeting ribonucleic acid” or “a targeting RNA” is a ribonucleic acid complementary to a region of the targeted mRNA.

In certain embodiments, the editing agent comprises a targeting ribonucleic acid complementary to a region of the targeted mRNA transcript.

In certain embodiments, the targeting ribonucleic acid is a guide RNA. In certain embodiments, the targeting ribonucleic acid is a trigger RNA.

In certain embodiments, the trigger RNA is an adRNA or a cadRNA.

The term “adRNA,” as used herein, refers to ADAR-recruiting guide. adRNAs comprise a programmable antisense region that is complementary to the target RNA sequence with a mismatched cytidine opposite the target adenosine. Additionally, they comprise zero, one or two ADAR-recruiting domains engineered from the naturally occurring ADAR substrate GluR2 pre-mRNA, see, e.g., Katrekar et al., Nat Methods. 2019 March; 16(3): 239-242, the content of which is incorporated by reference in its entirety.

The term “cadRNA,” as used herein, refers to a circular ADAR-recruiting RNA. Like adRNAs, cadRNAs contain recruiting domains that are derived from native RNA sites known to be heavily edited by ADARs which recruit endogenous ADARs to target sites, see, e.g., Katrekar et al., Nat Biotechnol. 2022 June; 40(6):938-945), the content of which is incorporated by reference in its entirety.

In certain embodiments, the trigger RNA comprises two domains: (1) a domain that shares homology to a target nucleic acid (e.g., and directs binding of a deaminase to the target; and (2) a domain that binds a deaminase enzyme. In certain embodiments, the targeting ribonucleic acid is circular. In certain embodiments, the targeting ribonucleic acid is linear.

In certain embodiments, the targeting ribonucleic acid effects the base alteration via binding to an endogenous deaminase domain (e.g., an adenosine deaminase or cytidine deaminase).

In certain embodiments, the base editor system comprises a targeting ribonucleic acid complementary to the region of the mRNA transcript; and a base editor comprising a programmable RNA binding domain and an adenosine deaminase domain or a cytidine deaminase domain, wherein the polynucleotide programmable RNA binding domain in conjunction with the ribonucleic acid binds to the region of the mRNA transcript to effect the base alteration.

In certain embodiments, the polynucleotide programmable RNA binding domain comprises a nuclease inactive variant of Cas13 or a nickase variant of Cas13. Nickase variants of Cas13 are known in the art. For example, those described in WO2019/005884 the contents of which are incorporated herein in their entirety.

In certain embodiments, the cytidine deaminase domain is selected from the group consisting of the apolipoprotein B mRNA-editing enzyme, catalytic polypeptide-like (APOBEC) family of deaminases, such as APOBEC1, APOBEC2, APOBEC3A, APOBEC3B, APOBEC3C, APOBEC3D/E, APOBEC3F, APOBEC3G, APOBEC3H, or APOBEC4; activation-induced cytidine deaminase (AID), such as activation induced cytidine deaminase (AICDA); cytosine deaminase 1 (CDA1) or CDA2; or cytosine deaminase acting on tRNA (CDAT).

In certain embodiments, the adenosine deaminase is selected from the group consisting of adenosine deaminase acting on RNA 1 (ADAR1), ADAR2, ADAR3; adenosine deaminase acting on tRNA 1 (ADAT1), ADAT2, ADAT3; and naturally occurring or engineered tRNA-specific adenosine deaminase (TadA).

Exemplary base editors that may be utilized to achieve the methods of the invention can include, for example, those described in the following references and/or patent publications, each of which is incorporated by reference in its entirety: (a) WO2015/089406 and its equivalents in the US or around the world; (b) WO2017/070632 and its equivalents in the US or around the world; (c) WO2017/070633 and its equivalent in the US or around the world; (d) WO2018/027078 and its equivalents in the US or around the world; (e) WO2018/071868 and its equivalents in the US or around the world; (f) WO2017/048390 and its equivalents in the US or around the world; (f) WO2018/119359 and its equivalents in the US or around the world; (g) WO2018/119354 and its equivalents in the US or around the world; (h) WO2018/031683 and its equivalents in the US or around the world; (i) WO2018/176009 and its equivalents in the US or around the world; (j) WO2018/021878 and its equivalents in the US and around the world; (k) WO2019/060746 and its equivalents in the US and around the world; (1) WO2020/160517 and its equivalents in the US and around the world; (m) WO2020/168132 and its equivalents in the US and around the world; (n) WO2020/028823 and its equivalents in the US and around the world; (o) WO2019/226953 and its equivalents in the US and around the world; (p) WO2019/005884 and its equivalents in the US and around the world; (q) Komor, A. C., Kim, Y. B., Packer, M. S., Zuris, J. A. & Liu, D. R. Programmable editing of a target base in genomic DNA without double-stranded DNA cleavage. Nature 533, 420-(2016); (r) Gaudelli, N. M. et al. Programmable base editing of A.T to G.C in genomic DNA without DNA cleavage. Nature 551, 464-(2017); (s) any of the references listed in this specification which reports or describes a base editor known in the art.

The term “target site” refers to a sequence within a nucleic acid molecule that is deaminated by a deaminase or a fusion protein comprising a deaminase

Some aspects of the disclosure are based on the recognition that any of the base editors provided herein are capable of efficiently generating an intended mutation in a nucleic acid (e.g., a nucleic acid located on an episomal vector) without generating a significant number of unintended mutations. In some embodiments, an intended mutation is a mutation that is generated by a specific base editor bound to a gRNA, specifically designed to alter an intended mutation. In some embodiments, the intended mutation is a mutation that generates a stop codon, for example, a premature stop codon within the coding region of a gene. In some embodiments, the intended mutation is a mutation that eliminates a stop codon. In some embodiments, the intended mutation is a mutation that alters the splicing of a gene. In some embodiments, the intended mutation is a mutation that alters the regulatory sequence of a gene (e.g., a gene promotor or gene repressor).

In certain embodiments, the base alteration results in a conversion of the amino acid codon to the premature stop codon, preferably upstream of a splice junction, to thereby down-regulate the expression of the gene. A “splice junction” as used herein includes the region in a mature mRNA transcript or the encoded polypeptide where the 3′ end of a first exon joins with the 5′ end of a second exon. The size of the region may vary, and may include 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100 or more (including all integers in between) nucleotide or amino acid residues on either side of the exact residues where the 3′ end of one exon joins with the 5′ end of another exon. An “exon” refers to a nucleic acid sequence that is represented in the mature form of an RNA molecule after either portion of a precursor RNA (introns) have been removed by cis-splicing or two or more precursor RNA molecules have been ligated by trans-splicing.

In certain embodiments, the base alteration results in the conversion of a CGA, CAG, or TGG codon to a premature TGA, TAG, or TAA stop codon, respectively, and the base editor comprises the cytidine deaminase domain, preferably, the CAG codon is located in proximity to the 5′-end of the gene.

In certain embodiments, the base alteration results in a conversion of a premature stop codon to an amino acid codon to thereby up-regulate the expression of the gene.

In certain embodiments, the base alteration results in the conversion of a premature UAG, UAA, or UGA stop codon to a CAG, CAA, or CGA, respectively and the base editor comprises the adenosine deaminase domain, preferably, the premature UAG stop codon is located in proximity to the 5′-end of the gene.

### Vectors

In certain embodiments, the method of the invention further comprises administering to the subject the episomal vector comprising the gene.

In certain embodiments, the episomal vector is a nonviral vector, including but not limited to a plasmid.

In certain embodiments, the episomal vector is a viral vector. Examples of viral vectors include, but are not limited to, an adeno-associated viral (AAV) vector, a lentivirus vector, and an adenovirus vector.

A vector nucleic acid sequence generally contains at least an origin of replication for propagation in a cell and optionally additional elements, such as a heterologous polynucleotide sequence, expression control element (e.g., a promoter, enhancer), intron, an inverted terminal repeat (ITR), selectable marker (e.g., antibiotic resistance), polyadenylation signal.

The term “expression cassette”, as used herein, refers to a nucleic acid construct comprising nucleic acid elements sufficient for the expression of a polynucleotide molecule. Typically, an expression cassette comprises a polynucleotide molecule operably linked to a promoter sequence.

An “expression control element” refers to nucleic acid sequence(s) that influence expression of an operably linked nucleic acid. Expression control elements as set forth herein include promoters and enhancers. Vector sequences, including AAV vectors and non-viral vectors, can include one or more “expression control elements.” Typically, such elements are included to facilitate proper heterologous polynucleotide transcription and as appropriate translation (e.g., a promoter, enhancer, splicing signal for introns, maintenance of the correct reading frame of the gene to permit in-frame translation of mRNA and, stop codons etc.).

Such elements typically act in cis, referred to as a “cis acting” element, but can also act in trans.

Expression control can be affected at the level of transcription, translation, splicing, message stability, etc. Typically, an expression control element that modulates transcription is juxtaposed near the 5′ end (i.e., “upstream”) of a transcribed nucleic acid. Expression control elements can also be located at the 3′ end (i.e., “downstream”) of the transcribed sequence or within the transcript (e.g., in an intron). Expression control elements can be located adjacent to or at a distance away from the transcribed sequence (e.g., 1-10, 10-25, 25-50, 50-100, 100-500, or more nucleotides from the polynucleotide), even at considerable distances. Nevertheless, owing to the length limitations of AAV vectors, expression control elements in AAV vectors will typically be within 1 to 1000 nucleotides from the transcription start site of the heterologous nucleic acid.

Functionally, expression of an operably linked nucleic acid is at least in part controllable by the element (e.g., promoter) such that the element modulates transcription of the nucleic acid and, as appropriate, translation of the transcript. A specific example of an expression control element is a promoter, which is usually located 5′ of the transcribed nucleic acid sequence. A promoter typically increases an amount expressed from operably linked nucleic acid as compared to an amount expressed when no promoter exists.

The term “operably linked” means that the regulatory sequences necessary for expression of a nucleic acid sequence are placed in the appropriate positions relative to the sequence so as to mediate expression of the nucleic acid sequence. This same definition is sometimes applied to the arrangement of nucleic acid sequences and transcription control elements (e.g., promoters, enhancers, and termination elements) in an expression vector, e.g., rAAV vector or non-viral vector. Encoding sequences can be operably linked to regulatory sequences in sense or antisense orientation. In certain embodiments, the promoter is a heterologous promoter.

The term “heterologous promoter”, as used herein, refers to a promoter that is not found to be operably linked to a given encoding sequence in nature. In certain embodiments, an expression cassette can comprise additional elements, for example, an intron, an enhancer, a polyadenylation site, a woodchuck response element (WRE), and/or other elements known to affect expression levels of the encoding sequence.

As used herein, the term “promoter” refers to a nucleotide sequence capable of controlling the expression of a coding sequence or functional RNA. In general, nucleic acid molecules of the instant invention are located 3′ of a promoter sequence. In certain embodiments, a promoter sequence consists of proximal and more distal upstream elements and can comprise an enhancer element.

An “enhancer” as used herein can refer to a sequence that is located adjacent to the heterologous nucleic acid. Enhancer elements are typically located upstream of a promoter element but also function and can be located downstream of or within a sequence. Hence, an enhancer element can be located 10-50 base pairs, 50-100 base pairs, 100-200 base pairs, or 200-300 base pairs, or more base pairs upstream or downstream of a heterologous nucleic acid sequence. Enhancer elements typically increase expression of an operably linked nucleic acid afforded by a promoter element.

An expression construct can comprise regulatory elements which serve to drive expression in a particular cell or tissue type. Expression control elements (e.g., promoters) include those active in a particular tissue or cell type, referred to herein as a “tissue-specific expression control element/promoter.” Tissue-specific expression control elements are typically active in specific cell or tissue (e.g., liver). Expression control elements are typically active in particular cells, tissues or organs because they are recognized by transcriptional activator proteins, or other regulators of transcription, that are unique to a specific cell, tissue or organ type. Such regulatory elements are known to those of skill in the art (see, e.g., Green, M. and Sambrook, J. (2012) Molecular Cloning: A Laboratory Manual. 4th Edition, Vol. II, Cold Spring Harbor Laboratory Press, New York; and Ausubel et al. (2010) Current protocols in molecular biology, John Wiley & Sons, New York).

The incorporation of tissue specific regulatory elements in the expression constructs provides for at least partial tissue tropism for the expression of a heterologous nucleic acid encoding a protein or inhibitory RNA. Examples of promoters that are active in liver are the transthyretin (TTR) gene promoter; human alpha 1-antitrypsin (hAAT) promoter; the apolipoprotein A-I promoter; albumin, Miyatake, et al., J. Virol., 71:5124-32 (1997); hepatitis B virus core promoter, Sandig, et al., Gene Ther. 3: 1002-9 (1996); alpha-fetoprotein (AFP), Arbuthnot, et al., Hum. Gene. Ther., 7:1503-14 (1996); human Factor IX promoter; thyroxin binding globulin (TBG) promoter; TTR minimal enhancer/promoter; alpha-antitrypsin promoter; LSP (845 nt) (requires intronless scAAV); and LSP1 promoter, among others. An example of an enhancer active in liver is apolipoprotein E (apoE) HCR-1 and HCR-2 (Allan et al., J. Biol. Chem., 272:29113-19 (1997)).

Expression control elements also include ubiquitous or promiscuous promoters/enhancers which are capable of driving expression of a polynucleotide in many different cell types. Such elements include, but are not limited to the cytomegalovirus (CMV) immediate early promoter/enhancer sequences, the Rous sarcoma virus (RSV) promoter/enhancer sequences and the other viral promoters/enhancers active in a variety of mammalian cell types, or synthetic elements that are not present in nature (see, e.g., Boshart et al., Cell, 41:521-530 (1985)), the SV40 promoter, the dihydrofolate reductase promoter, the cytoplasmic b-actin promoter and the phosphoglycerate kinase (PGK) promoter.

Expression control elements also can confer expression in a manner that is regulatable, that is, a signal or stimuli increases or decreases expression of the operably linked heterologous polynucleotide. A regulatable element that increases expression of the operably linked polynucleotide in response to a signal or stimuli is also referred to as an “inducible element” (i.e., is induced by a signal). Particular examples include, but are not limited to, a hormone (e.g., steroid) inducible promoter. Typically, the amount of increase or decrease conferred by such elements is proportional to the amount of signal or stimuli present; the greater the amount of signal or stimuli, the greater the increase or decrease in expression. Particular non-limiting examples include zinc-inducible sheep metallothionine (MT) promoter; the steroid hormone-inducible mouse mammary tumor virus (MMTV) promoter; the T7 polymerase promoter system (WO 98/10088); the tetracycline-repressible system (Gossen, et al., Proc. Natl. Acad. Sci. USA, 89:5547-5551 (1992)); the tetracycline-inducible system (Gossen, et al., Science. 268: 1766-1769 (1995); see also Harvey, et al., Curr. Opin. Chem. Biol. 2:512-518 (1998)); the RU486-inducible system (Wang, et al., Nat. Biotech. 15:239-243 (1997) and Wang, et al., Gene Ther. 4:432-441 (1997)]; and the rapamycin-inducible system (Magari, et al., J. Clin. Invest. 100:2865-2872 (1997); Rivera, et al., Nat. Medicine. 2:1028-1032 (1996)). Other regulatable control elements which can be useful in this context are those which are regulated by a specific physiological state, e.g., temperature, acute phase, development.

Other examples of promoters include, but are not limited to, the phosphoglycerate kinase (PKG) promoter, CAG (composite of the CMV enhancer, the chicken beta actin promoter (CBA) and the rabbit beta globin intron) and other constitutive promoters, NSE (neuronal specific enolase), synapsin or NeuN promoters, the SV40 early promoter, mouse mammary tumor virus LTR promoter; adenovirus major late promoter (Ad MLP); a herpes simplex virus (HSV) promoter, SFFV promoter, rous sarcoma virus (RSV) promoter, rat insulin promoter, TBG promoter and other liver-specific promoters, the desmin promoter and similar muscle-specific promoters, the EF1-alpha promoter, synthetic promoters, hybrid promoters, promoters with multi-tissue specificity, and the like, all of which are promoters well known and readily available to those of skill in the art. Other promoters can be of human origin or from other species, including from mice.

Expression control elements also include the native elements(s) for the heterologous polynucleotide. A native control element (e.g., promoter) can be used when it is desired that expression of the heterologous polynucleotide should mimic the native expression. The native element can be used when expression of the heterologous polynucleotide is to be regulated temporally or developmentally, or in a tissue-specific manner, or in response to specific transcriptional stimuli. Other native expression control elements, such as introns, polyadenylation sites or Kozak consensus sequences can also be used.

In the example of an expression control element in operable linkage with a nucleic acid, the relationship is such that the control element modulates expression of the nucleic acid. More specifically, for example, two DNA sequences operably linked means that the two DNAs are arranged (cis or trans) in such a relationship that at least one of the DNA sequences is able to exert a physiological effect upon the other sequence.

Accordingly, additional elements for vectors include, without limitation, an expression control (e.g., promoter/enhancer) element, a transcription termination signal or stop codon, 5′ or 3′ untranslated regions (e.g., polyadenylation (poly A) sequences) which flank a sequence, such as one or more copies of an AAV ITR sequence, or an intron.

Further elements include, for example, filler or stuffer polynucleotide sequences, for example to improve packaging and reduce the presence of contaminating nucleic acid. AAV vectors typically accept inserts of DNA having a size range which is generally about 4 kb to about 5.2 kb, or slightly more. Thus, for shorter sequences, inclusion of a stuffer or filler in order to adjust the length to near or at the normal size of the virus genomic sequence acceptable for AAV vector packaging into virus particle. In certain embodiments, a filler/stuffer nucleic acid sequence is an untranslated (non-protein encoding) segment of nucleic acid. For a nucleic acid sequence less than 4.7 kb, the filler or stuffer polynucleotide sequence has a length that when combined (e.g., inserted into a vector) with the sequence has a total length between about 3.0-5.5 kb, or between about 4.0-5.0 kb, or between about 4.3-4.8 kb.

As used herein, the term “gene transfer system” refers to any means of delivering a composition comprising a nucleic acid sequence to a cell or tissue. For example, a gene transfer system can be a viral gene transfer system, e.g., intact viruses, modified viruses and VLPs to facilitate delivery of a viral vector to a desired cell or tissue. A gene transfer system can also be a non-viral delivery system that does not comprise viral coat protein or form a viral particle or VLP, e.g., liposome-based systems, polymer-based systems, protein-based systems, metallic particle-based systems, peptide cage systems, etc.

A viral vector is derived from or based upon one or more nucleic acid elements that comprise a viral genome. Particular viral vectors include retroviral, lentiviral and adeno-associated virus (AAV) vectors.

Retroviruses are enveloped, single-stranded RNA viruses comprising 5′ and 3′ LTRs, and a signal packaging sequence located just outside of the LTR. Different types of retrovirus vectors can contain different amounts of viral genome. In certain embodiments, the retrovirus vector is a lentiviral vector based on HIV, retaining all cis-acting sequences needed for viral RNA packaging, reverse transcription and proviral DNA integration, while removing all HIV protein-coding genes. Lentiviral vectors have a packaging capacity of up to about 9 kb. If needed, stuffer sequence can be used to increase rAAV nucleic acid size and packaging efficiency. Lentiviral vectors can be produced by supplying viral proteins needed for vector production in trans using appropriate plasmids and cell lines. (Bulcha et a., (2021) Sig. Transduct. Target Ther. 6:53.)

The term “recombinant,” as a modifier of vector, such as recombinant AAV (rAAV) vector, as well as a modifier of sequences such as recombinant polynucleotides and polypeptides, means that the compositions have been manipulated (i.e., engineered) in a fashion that generally does not occur in nature. Although the term “recombinant” is not always used herein in reference to AAV vectors, as well as sequences such as polynucleotides, recombinant forms including polynucleotides, are expressly included in spite of any such omission.

Recombinant adeno-associated viral vector (also referred to herein as “rAAV”) are based on the adeno-associated virus. The adeno-associated virus is a single-strand DNA virus containing a 4.7-kb genome flanked by 145-nt ITRs on both ends of the genome. ITR activity is important for self-priming and packaging, and may also provide additional activity such as promoter activity.

An rAAV contains AAV recombinant nucleic acid and a viral capsid. The rAAV recombinant nucleic acid lacks one or more AAV proteins involved in viral replication. Recombinant adeno-associated viral vectors typically accept inserts of DNA having a size range generally about 4 kb to about 5.2 kb. If needed, stuffer sequence can be used to increase rAAV nucleic acid size and packaging efficiency. In different embodiments, the rAAV nucleic acid including stuffer is 4-5.2kb, 3.0-5.5 kb, 4.0-5.0 kb, 4.3-4.8 kb, about 4.2 kb, about 4.3 kb, about 4.4 kb about 4.5 kb, about 4.6 kb, or about 4.7 kb. Preferred stuffer sequences avoid coding sequences, repetitive sequences, recombination sequences, and immunogenic sequences.

In certain embodiments rAAV nucleic acid comprise a 5′ ITR and/or 3′ ITR independently selected from 5′ and 3′ ITRs provided in AAV1, AAV2, AAV3, AAV4, AAV5, AAV6, AAV7, AAV8, AAV9, AAV10, AAV11, AAV12, AAVrh.10, AAVrh.74 and AAV3B ITRs. In further embodiments 5′ and 3′ ITRs are present, and both ITRs are from the same serotype genome.

Naturally occurring AAV capsids contain viral protein VP1, VP2 and VP3 in a ratio of about 1:1:10. AAV vectors can be produced where all three viral proteins are based upon a particular serotypes or where one, two or all three viral protein are based on different serotypes or variants thereof.

Different serotypes exist within different types of viruses. The different serotypes can provide for different activities, such as cell or tissue tropism and likelihood of generating a host immune response. The term “serotype” broadly refers to both serologically distinct viruses as well as viruses not serologically distinct that can be within a subgroup or a variant of a given serotype. Serologic distinctiveness can be determined based on the lack of cross-reactivity between antibodies to one capsid as compared to another capsid. Such cross-reactivity differences are usually due to differences in capsid protein sequences/antigenic determinants (e.g., due to VP1, VP2, and/or VP3 sequence differences of AAV serotypes).

As more naturally occurring virus isolates are discovered or capsid mutants generated, there may or may not be serological differences with any of the currently existing serotypes. Thus, in cases where the new virus has no serological difference, this new virus would be a subgroup or variant of the corresponding serotype.

In certain embodiments, AAV capsids are based on VP1, VP2 or VP3 having a sequence identity of at least 80% to a VP1, VP2 or VP3 of any of AAV1, AAV2, AAV3, AAV4, AAV5, AAV6, AAV7, AAV8, AAV9, AAV10, AAV11, AAV12, AAVrh.74, AAV3B, AAV-2i8, AAVrh.10, AAVrh.8, AAVHSC, AAV-B1, AAV-AS, AAV1/rh.10, SEQ ID NO: 1 and SEQ ID NO: 2; as well as variants (e.g., capsid variants, such as amino acid insertions, additions, substitutions and deletions) thereof (See for example, U.S. Pat. Nos. 9,909,142 and 9,840,719 disclosing RHM4-1, RHM15-1, RHM15-2, RHM15-3/RHM15-5, RHM15-4 and RHM15-6; U.S. Patent Publication No. 2013/0059732 and U.S. Pat. No. 9,169,299, disclosing LK01, LK02, and LK03; and U.S. Pat. No. 11,110,153; the disclosures of which are herein incorporated in their entirety).

Recombinant AAV capsid and nucleic acid can be based on the same serotype (or subgroup or variant), or can be different from each other. In certain embodiments, an rAAV nucleic acid has the same serotype genome (e.g., ITRs) as the encapsidating capsid protein.

In different embodiments, the rAAV capsid comprises a protein having a sequence at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.1%, at least 99.2%, at least 99.3%, at least at least 99.4%, at least 99.5%, at least 99.9% or 100% identical to a VP1, VP2 or VP3 of any of AAV1, AAV2, AAV3, AAV4, AAV5, AAV6, AAV7, AAV8, AAV9, AAV10, AAV11, AAV12, AAVrh.74, AAV3B, AAV-2i8, AAVrh.10, AAVrh.8, AAVHSC, AAV-B1, AAV-AS, AAV1/rh.10; and VP1 of SEQ ID NO: 1 or SEQ ID NO: 2.

TABLE 3
Exemplary AAV capsid protein sequences

| SEQ ID | |
|---|---|
| NO: | Amino Acids Sequence |
| 1 | MAADGYLPDWLEDNLSEGIREWWDLKPGAPKPKANQQKQDNGRGLVL |
| | PGYKYLGPFNGLDKGEPVNAADAAALEHDKAYDQQLQAGDNPYLRYN |
| | HADAEFQERLQEDTSFGGNLGRAVFQAKKRVLEPLGLVESPVKTAPGK |
| | KRPVEPSPQRSPDSSTGIGKKGQQPAKKRLNFGQTGDSESVPDPQPIGEPP |
| | AGPSGLGSGTMAAGGGAPMADNNEGADGVGSSSGNWHCDSTWLGDR |
| | VITTSTRTWALPTYNNHLYKQISNGTSGGSTNDNTYFGYSTPWGYFDFN |
| | RFHCHFSPRDWQRLINNNWGFRPKRLNFKLFNIQVKEVTQNEGTKTIAN |
| | NLTSTIQVFTDSEYQLPYVLGSAHQGCLPPFPADVFMIPQYGYLTLNNGS |
| | QAVGRSSFYCLEYFPSQMLRTGNNFEFSYNFEDVPFHSSYAHSQSLDRL |
| | MNPLIDQYLYYLSRTQSTGGTAGTQQLLFSQAGPNNMSAQAKNWLPGP |
| | CYRQQRVSTTLSQNNNSNFAWTGATKYHLNGRDSLVNPGVAMATHKD |
| | DEERFFPSSGVLMFGKQGAGKDNVDYSSVMLTSEEEIKTTNPVATEQYG |
| | VVADNLQQQNAAPIVGAVNSQGALPGMVWQNRDVYLQGPIWAKIPHT |
| | DGNFHPSPLMGGFGLKHPPPQILIKNTPVPADPPTTFNQAKLASFITQYST |
| | GQVSVEIEWELQKENSKRWNPEIQYTSNYYKSTNVDFAVNTEGTYSEPR |
| | PIGTRYLTRNL |
| 2 | MAADGYLPDWLEDNLSEGIREWWALQPGAPKPKANQQHQDNARGLVL |
| | PGYKYLGPGNGLDKGEPVNAADAAALEHDKAYDQQLKAGDNPYLKY |
| | NHADAEFQERLKEDTSFGGNLGRAVFQAKKRLLEPLGLVEEAAKTAPG |
| | KKRPVDQSPQEPDSSSGVGKSGKQPARKRLNFGQTGDSESVPDPQPLGE |
| | PPAAPTSLGSNTMASGGGAPMADNNEGADGVGNSSGNWHCDSQWLGD |
| | RVITTSTRTWALPTYNNHLYKQISSQSGASNDNHYFGYSTPWGYFDFNR |
| | FHCHFSPRDWQRLINNNWGFRPKKLSFKLFNIQVKEVTQNDGTTTIANN |
| | LTSTVQVFTDSEYQLPYVLGSAHQGCLPPFPADVFMVPQYGYLTLNNGS |
| | QAVGRSSFYCLEYFPSQMLRTGNNFQFSYTFEDVPFHSSYAHSQSLDRL |
| | MNPLIDQYLYYLNRTQGTTSGTTNQSRLLFSQAGPQSMSLQARNWLPGP |
| | CYRQQRLSKTANDNNNSNFPWTAASKYHLNGRDSLVNPGPAMASHKD |
| | DEEKFFPMHGNLIFGKEGTTASNAELDNVMITDEEEIRTTNPVATEQYGT |
| | VANNLQSSNTAPTTRTVNDQGALPGMVWQDRDVYLQGPIWAKIPHTD |
| | GHFHPSPLMGGFGLKHPPPQIMIKNTPVPANPPTTFSPAKFASFITQYSTG |
| | QVSVEIEWELQKENSKRWNPEIQYTSNYNKSVNVDFTVDINGVYSEPRP |
| | IGTRYLTRPL |

Recombinant AAV can be produced from different types of cell lines. In certain embodiments human HEK293 cells are used (American Type Culture Collection Accession Number ATCC CRL1573). Other host cell lines appropriate for rAAV production are described in, for example, Robert et al., (2017) Biotechnol. J., 12: 1600193; and International Application PCT/US2017/024951, the disclosures of which are herein incorporated in its entirety.

In certain embodiments, AAV helper functions are introduced into the host cell by transfecting the host cell with an AAV helper construct either prior to, or concurrently with, the transfection of an AAV expression vector. A host cell having AAV helper functions can be referred to as a “helper cell” or “packaging helper cell.” AAV helper constructs are thus sometimes used to provide at least transient expression of AAV rep and/or cap genes to complement missing AAV functions necessary for productive AAV transduction. AAV helper constructs often lack AAV ITRs and can neither replicate nor package themselves. These constructs can be, for example, in the form of a plasmid, phage, transposon, cosmid, virus, or virion. A number of AAV helper constructs have been described, such as the commonly used plasmids pAAV/Ad and pIM29+45 which encode both Rep and Cap expression products. A number of other vectors are known which encode Rep and/or Cap expression products. Recombinant AAV can be produced, for example, as described in U.S. Pat. No. 9,408,904; and International Applications PCT/US2017/025396 and PCT/US2016/064414, the disclosures of which are herein incorporated in their entirety.

The term “isolated,” when used as a modifier of a composition, means that the compositions are made by the hand of man or are separated, completely or at least in part, from their naturally occurring in vivo environment. Generally, isolated compositions are substantially free of one or more materials with which they normally associate with in nature, for example, one or more protein, nucleic acid, lipid, carbohydrate, or cell membrane.

The term “isolated” does not exclude combinations produced by the hand of man, for example, a rAAV sequence, or rAAV particle that packages or encapsidates an AAV vector genome (vg) and a pharmaceutical formulation. The term “isolated” also does not exclude alternative physical forms of the composition, such as hybrids/chimeras, multimers/oligomers, modifications (e.g., phosphorylation, glycosylation, lipidation) or derivatized forms, or forms expressed in host cells produced by the hand of man.

The term “substantially pure” refers to a preparation comprising at least 50-60% by weight the compound of interest (e.g., nucleic acid, oligonucleotide, protein, etc.). The preparation can comprise at least 75% by weight, or at least 85% by weight, or about 90-99% by weight, of the compound of interest. Purity is measured by methods appropriate for the compound of interest (e.g., chromatographic methods, agarose or polyacrylamide gel electrophoresis, HPLC analysis, and the like).

### Therapeutic Proteins

The episomal vector can deliver a variety of different genes that can be expressed to provide a protein having a desired activity. Examples of genes include those providing a healthy copy of gene in a subject where the gene is defective or a new, a modified gene that can help treat a disease or disorder, or a new gene encoding for protein providing a beneficial effect.

In different embodiments, a gene encodes GAA (acid alpha-glucosidase) for treatment of Pompe disease; TPP1 (tripeptidyl peptidase-1) for treatment of late infantile neuronal ceroid lipofuscinosis type 2 (CLN2); ATP7B (copper transporting ATPase2) for treatment of Wilson's disease; alpha galactosidase for treatment of Fabry disease; ASS1 (arginosuccinate synthase) for treatment of Citrullinemia Type 1; beta-glucocerebrosidase for treatment of Gaucher disease Type 1; beta-hexosaminidase A for treatment of Tay-Sachs disease; SERPING1 (C1 protease inhibitor or C1 esterase inhibitor) for treatment of hereditary angioedema (HAE), also known as C1 inhibitor deficiency type I and type II); or glucose-6-phosphatase for treatment of glycogen storage disease type I (GSDI).

In different embodiments, the gene encodes insulin, glucagon, growth hormone (GH), parathyroid hormone (PTH), growth hormone releasing factor (GRF), follicle stimulating hormone (FSH), luteinizing hormone (LH), human chorionic gonadotropin (hCG), vascular endothelial growth factor (VEGF), angiopoietins, angiostatin, granulocyte colony stimulating factor (GCSF), erythropoietin (EPO), connective tissue growth factor (CTGF), basic fibroblast growth factor (bFGF), acidic fibroblast growth factor (aFGF), epidermal growth factor (EGF), transforming growth factor α (TGFα), platelet-derived growth factor (PDGF), insulin growth factors I or II (IGF-I or IGF-II), TGFβ, activins, bone morphogenic protein (BMP), nerve growth factor (NGF), brain-derived neurotrophic factor (BDNF), neurotrophins NT-3 or NT4/5, ciliary neurotrophic factor (CNTF), glial cell line derived neurotrophic factor (GDNF), neurturin, agrin, netrin-1 or netrin-2, hepatocyte growth factor (HGF), ephrins, noggin, sonic hedgehog or tyrosine hydroxylase.

In different embodiments, the gene encodes thrombopoietin (TPO), an interleukin (IL-1 through IL-36), monocyte chemoattractant protein, leukemia inhibitory factor, granulocyte-macrophage colony stimulating factor, Fas ligand, tumor necrosis factors α or β, interferons α, β, or γ, stem cell factor, flk-2/flt3 ligand, IgG, IgM, IgA, IgD or IgE, chimeric immunoglobulins, an antibody, humanized antibodies, single chain antibodies, T cell receptors, chimeric T cell receptors, single chain T cell receptors, class I or class II MHC molecules. Antibodies and immunoglobulins can, for example, be provided targeting cancer cells or other disease or disorder causing cells.

In different embodiments, the gene encodes CFTR (cystic fibrosis transmembrane regulator protein), a blood coagulation (clotting) factor (Factor XIII, Factor IX (FIX), Factor VIII (FVIII), Factor X, Factor VII, Factor VIIa, or protein C) a gain of function blood coagulation factor, erythropoietin, LDL receptor, lipoprotein lipase, ornithine transcarbamylase, β-globin, α-globin, spectrin, α-antitrypsin, adenosine deaminase (ADA), a metal transporter (ATP7A or ATP7), sulfamidase, an enzyme involved in lysosomal storage disease (ARSA), hypoxanthine guanine phosphoribosyl transferase, β-25 glucocerebrosidase, sphingomyelinase, lysosomal hexosaminidase, branched-chain keto acid dehydrogenase, a hormone, a growth factor, insulin-like growth factor 1 or 2, platelet derived growth factor, epidermal growth factor, nerve growth factor, neurotrophic factor −3 and −4, brain-derived neurotrophic factor, glial derived growth factor, transforming growth factor α and β, a cytokine, α-interferon, β-interferon, interferon-γ, interleukin-2, interleukin-4, interleukin 12, granulocyte-macrophage colony stimulating factor, lymphotoxin, a suicide gene product, herpes simplex virus thymidine kinase, cytosine deaminase, diphtheria toxin, cytochrome P450, deoxycytidine kinase, tumor necrosis factor, a drug resistance protein, a tumor suppressor protein (e.g., p53, Rb, Wt-1, NF1, Von Hippel-Lindau (VHL), adenomatous polyposis coli (APC)), a peptide with immunomodulatory properties, a tolerogenic or immunogenic peptide or protein Tregitope or hCDR1, insulin, glucokinase, guanylate cyclase 2D (LCA-GUCY2D), retinal pigment epithelium-specific 65 kDa protein (RPE65), Rab escort protein 1 (choroideremia), LCA 5 (LCA-lebercilin), ornithine ketoacid aminotransferase (gyrate atrophy), retinoschisin 1 (X-linked retinoschisis), X-linked retinitis pigmentosa GTPase (XLRP), MER proto-oncogene tyrosine kinase (MERTK) (autosomal recessive (AR) forms of retinitis pigmentosa (RP)), ABCA4 (Stargardt), ACHM 2, 3 and 4 (achromatopsia), an anti-vascular endothelial growth factor (VEGF) agent polypeptide (e.g., bevacizumab, brolucizumab, ranibizumab, aflibercept), DFNB1 (connexin 26 deafness), USH1C (Usher's syndrome 1C), PKD-1 or PKD-2 (polycystic kidney disease), TPP1 (tripeptidyl peptidase-1), a sulfatase, N-acetylglucosamine-1-phosphate transferase, cathepsin A, GM2-AP, NPC1, VPC2, a sphingolipid activator protein, or one or more donor sequences used as repair templates for genome editing.

In different embodiments, the gene encodes erythropoietin (EPO) for treatment of anemia; interferon-alpha, interferon-beta, and interferon-gamma for treatment of various immune disorders, viral infections and cancer; an interleukin (IL), including any one of IL-1 through IL-36, and corresponding receptors, for treatment of various inflammatory diseases or immuno-deficiencies; a chemokine, including chemokine (C-X-C motif) ligand 5 (CXCL5) for treatment of immune disorders; granulocyte-colony stimulating factor (G-CSF) for treatment of immune disorders such as Crohn's disease; granulocyte-macrophage colony stimulating factor (GM-CSF) for treatment of various human inflammatory diseases; macrophage colony stimulating factor (M-CSF) for treatment of various human inflammatory diseases; keratinocyte growth factor (KGF) for treatment of epithelial tissue damage; chemokines such as monocyte chemoattractant protein-1 (MCP-1) for treatment of recurrent miscarriage, HIV-related complications, and insulin resistance; tumor necrosis factor (TNF) and receptors for treatment of various immune disorders; alphal-antitrypsin for treatment of emphysema or chronic obstructive pulmonary disease (COPD); alpha-L-iduronidase for treatment of mucopolysaccharidosis I (MPS I); ornithine transcarbamoylase (OTC) for treatment of OTC deficiency; phenylalanine hydroxylase (PAH) or phenylalanine ammonia-lyase (PAL) for treatment of phenylketonuria (PKU); lipoprotein lipase for treatment of lipoprotein lipase deficiency; apolipoproteins for treatment of apolipoprotein (Apo) A-I deficiency; low-density lipoprotein receptor (LDL-R) for treatment of familial hypercholesterolemia (FH); albumin for treatment of hypoalbuminemia; lecithin cholesterol acyltransferase (LCAT); carbamoyl synthetase I; argininosuccinate synthetase; argininosuccinate lyase; arginase; fumarylacetoacetate hydrolase; porphobilinogen deaminase; cystathionine beta-synthase for treatment of homocystinuria; branched chain ketoacid decarboxylase; isovaleryl-CoA dehydrogenase; propionyl CoA carboxylase; methylmalonyl-CoA mutase; glutaryl CoA dehydrogenase; insulin; pyruvate carboxylase; hepatic phosphorylase; phosphorylase kinase; glycine decarboxylase; H-protein; T-protein; cystic fibrosis transmembrane regulator (CFTR); ATP-binding cassette, sub-family A (ABC1), member 4 (ABCA4) for the treatment of Stargardt disease; or dystrophin.

In a further embodiment the gene encodes a protein for treating a disease or disorder selected from the group consisting of: Hereditary angioedema, Pompe disease, hemophilia A, hemophilia B, Fabry disease, Huntington disease, Parkinson's disease, Alzheimer's disease, a synucleinopathy, epilepsy, neuropathic pain, wet macular degeneration, Usher 1F, Usher 1B, glaucoma, Leber congenital amaurosis, and Stargardt disease.

### Inhibitory Nucleic Acid

The episomal vector can provide a variety of different genes encoding for a variety of different inhibitory nucleic acid such as a short hairpin RNA (shRNA), a small interfering RNA (siRNA), a microRNA (miRNA), a RNAi, a ribozyme, and an antisense RNA. In different embodiments, the inhibitory nucleic acid binds to a gene, a transcript of a gene, or a transcript of a gene associated with a polynucleotide repeat disease selected from the group consisting of a huntingtin (HTT) gene, a gene associated with dentatorubropallidoluysian atrophy (atrophin 1, ATN1), androgen receptor on the X chromosome in spinobulbar muscular atrophy, human Ataxin-1, -2, -3, and -7, Cav2.1 P/Q voltage-dependent calcium channel (CACNA1A), TATA-binding protein, Ataxin 8 opposite strand (ATXN8OS), serine/threonine-protein phosphatase 2A 55 kDa regulatory subunit B beta isoform in spinocerebellar ataxia (type 1, 2, 3, 6, 7, 8, 12 17), FMR1 (fragile X mental retardation 1) in fragile X syndrome, FMR1 (fragile X mental retardation 1) in fragile X-associated tremor/ataxia syndrome, FMR1 (fragile X mental retardation 2) or AF4/FMR2 family member 2 in fragile XE mental retardation; myotonin-protein kinase (MT-PK) in myotonic dystrophy; Frataxin in Friedreich's ataxia; a mutant of superoxide dismutase 1 (SOD1) gene in amyotrophic lateral sclerosis; a gene involved in pathogenesis of Parkinson's disease and/or Alzheimer's disease; apolipoprotein B (APOB) and proprotein convertase subtilisin/kexin type 9 (PCSK9), hypercholesterolemia; HIV Tat, human immunodeficiency virus transactivator of transcription gene, in HIV infection; HIV TAR, HIV TAR, human immunodeficiency virus transactivator response element gene, in HIV infection; C-C chemokine receptor (CCR5) in HIV infection; Rous sarcoma virus (RSV) nucleocapsid protein in RSV infection, liver-specific microRNA (miR-122) in hepatitis C virus infection; p53, acute kidney injury or delayed graft function kidney transplant or kidney injury acute renal failure; protein kinase N3 (PKN3) in advance recurrent or metastatic solid malignancies; LMP2, LMP2 also known as proteasome subunit beta-type 9 (PSMB 9), metastatic melanoma; LMP7, also known as proteasome subunit beta-type 8 (PSMB 8), metastatic melanoma; MECL1 also known as proteasome subunit beta-type 10 (PSMB 10), metastatic melanoma; vascular endothelial growth factor (VEGF) in solid tumors; kinesin spindle protein in solid tumors, apoptosis suppressor B-cell CLL/lymphoma (BCL-2) in chronic myeloid leukemia; ribonucleotide reductase M2 (RRM2) in solid tumors; Furin in solid tumors; polo-like kinase 1 (PLK1) in liver tumors, diacylglycerol acyltransferase 1 (DGAT1) in hepatitis C infection, beta-catenin in familial adenomatous polyposis; beta2 adrenergic receptor, glaucoma; RTP801/Redd1 also known as DNA damage-inducible transcript 4 protein, in diabetic macular edema (DME) or age-related macular degeneration; vascular endothelial growth factor receptor I (VEGFR1) in age-related macular degeneration or choroidal neovascularization; caspase 2 in non-arteritic ischaemic optic neuropathy; keratin 6A N17K mutant protein in pachyonychia congenital; influenza A virus genome/gene sequences in influenza infection; severe acute respiratory syndrome (SARS) coronavirus genome/gene sequences in SARS infection; respiratory syncytial virus genome/gene sequences in respiratory syncytial virus infection; Ebola filovirus genome/gene sequence in Ebola infection; hepatitis B and C virus genome/gene sequences in hepatitis B and C infection; herpes simplex virus (HSV) genome/gene sequences in HSV infection; coxsackievirus B3 genome/gene sequences in coxsackievirus B3 infection; silencing of a pathogenic allele of a gene (allele-specific silencing) like torsin A (TOR1A) in primary dystonia, pan-class I and HLA-allele specific in transplant; and mutant rhodopsin gene (RHO) in autosomal dominantly inherited retinitis pigmentosa (adRP).

### Gene Editing

The episomal vector can provide a variety of different genes encoding for a variety of different gene editing nucleic acid such as ZFN, TALEN, and CRISPR-Cas9. In different embodiments the gene editing nucleic acid edits a subject's DNA to provide a therapeutic protein as provided supra., or disrupt a gene as provided supra.

### Delivery Systems

In certain embodiments, the base editor system or a component thereof is encoded by one or more nucleic acid molecules administered to the subject, preferably the ribonucleic acid and the base editor are encoded by one or more RNA molecules, such as one or more mRNA molecules, administered to the subject.

Any RNA of the systems, for example a guide RNA or a base editor-encoding mRNA, can be delivered in the form of RNA. Base editor-encoding mRNA can be generated using in vitro transcription. For example, nuclease mRNA can be synthesized using a PCR cassette containing the following elements: T7 promoter, optional kozak sequence (GCCACC), nuclease sequence, and 3′ UTR such as a 3′ UTR from beta globin-polyA tail. The cassette can be used for transcription by T7 polymerase. Guide polynucleotides (e.g., gRNA) can also be transcribed using in vitro transcription from a cassette containing a T7 promoter, followed by the sequence “GG”, and guide polynucleotide sequence. To enhance expression and reduce possible toxicity, the base editor-coding sequence and/or the guide nucleic acid can be modified to include one or more modified nucleoside e.g. using pseudo-U or 5-Methyl-C.

Nucleic acids encoding nucleobase editors according to the present disclosure can be administered to subjects or delivered into cells in vitro or in vivo by art-known methods or as described herein. In one embodiment, nucleobase editors can be delivered by, e.g., vectors (e.g., viral or non-viral vectors), non-vector based methods (e.g., using naked DNA, DNA complexes, lipid nanoparticles), or a combination thereof.

Nucleic acids encoding nucleobase editors can be delivered directly to cells as naked DNA or RNA, for instance by means of transfection or electroporation, or can be conjugated to molecules (e.g., N-acetylgalactosamine) promoting uptake by the target cells. Nucleic acid vectors, such as the vectors described herein can also be used.

Nucleic acid vectors can comprise one or more sequences encoding a domain of a base editing system described herein. A vector can also comprise a sequence encoding a signal peptide (e.g., for nuclear localization, nucleolar localization, or mitochondrial localization), associated with (e.g., inserted into or fused to) a sequence coding for a protein.

As one example, a nucleic acid vectors can include a Cas9 coding sequence that includes one or more nuclear localization sequences (e.g., a nuclear localization sequence from SV40), and an adenosine deaminase.

### Non-Viral Methods

In certain embodiments, the one or more nucleic acid molecules encoding a base editor system or component thereof of the invention is administered via a non-viral delivery system, including for example, encapsulated in a lipid nanoparticle (LNP).

In certain embodiments, the one or more nucleic acid molecules encoding a base editor system or component thereof of the invention are delivered or administered with a non-viral delivery system. Non-viral delivery systems include for example, chemical methods, such as non-viral vectors, or extracellular vesicles and physical methods, such as gene gun, electroporation, particle bombardment, ultrasound utilization and magnetofection.

In certain embodiments, the one or more nucleic acid molecules encoding a base editor system or component thereof of the invention are delivered as naked DNA, minicircles, transposons, of closed-ended linear duplex DNA.

In certain embodiments, the one or more nucleic acid molecules encoding a base editor system or component thereof of the invention are delivered or administered in AAV vector particles, or other viral particles, that are further encapsulated or complexed with liposomes, nanoparticles, lipid nanoparticles, polymers, microparticles, microcapsules, micelles, or extracellular vesicles.

In certain embodiments, the one or more nucleic acid molecules encoding a base editor system or component thereof of the invention are delivered or administered with non-viral vectors.

As used herein, a “non-viral vector” refers to a vector that is not delivered by viral particles or by viral-like particles (VLPs). According to certain embodiments, a non-viral vector is a vector that is not delivered by a capsid. The vector can be encapsulated, admixed, or otherwise associated with the non-viral delivery nanoparticle.

Any suitable non-viral delivery system known to those skilled in the art in view of the present disclosure can be used in the invention. The non-viral delivery nanoparticle can be, for example, a lipid-based nanoparticle, a polymer-based nanoparticle, a protein-based nanoparticle, a microparticle, a microcapsule, a metallic particle-based nanoparticle, a peptide cage nanoparticle, etc.

A non-viral delivery nanoparticle of the instant invention can be constructed by any method known in the art, and a non-viral vector of the instant invention can be constructed by any method known in the art.

### Lipid-Based Delivery Systems

Lipid-based delivery systems are well known in the art, and any suitable lipid-based delivery system known to those skilled in the art in view of the present disclosure can be used in the invention. Examples of lipid-based delivery systems include, e.g., liposomes, lipid nanoparticles, micelles, or extracellular vesicles.

A “lipid nanoparticle” or “LNP” refers to a lipid-based vesicle useful for delivery of AAV and non-viral vectors having dimensions on the nanoscale, i.e., from about 10 nm to about 1000 nm, or from about 50 to about 500 nm, or from about 75 to about 127 nm. Without being bound by theory, an LNP is believed to provide a polynucleotide, expression cassette, AAV vector, or non-viral vector with partial or complete shielding from the immune system. Shielding allows delivery of the polynucleotide, expression cassette, AAV vector, or non-viral vector to a tissue or cell while avoiding inducing a substantial immune response against the polynucleotide, expression cassette, AAV vector, or non-viral vector in vivo.

Shielding can also allow repeated administration without inducing a substantial immune response against the polynucleotide, expression vector, AAV vector, or non-viral vector in vivo (e.g., in a subject such as a human). Shielding can also improve or increase polynucleotide, expression cassette, AAV vector, or non-viral vector delivery efficiency in vivo.

The pI (isoelectric point) of AAV is in a pH range from about 6 to about 6.5. Thus, the AAV surface carries a slight negative charge. As such it can be beneficial for an LNP to comprise a cationic lipid such as, for example, an amino lipid. Exemplary amino lipids have been described in U.S. Pat. Nos. 9,352,042, 9,220,683, 9,186,325, 9,139,554, 9,126,966 9,018,187, 8,999,351, 8,722,082, 8,642,076, 8,569,256, 8,466,122, and 7,745,651 and U.S. Patent Publication Nos. 2016/0213785, 2016/0199485, 2015/0265708, 2014/0288146, 2013/0123338, 2013/0116307, 2013/0064894, 2012/0172411, and 2010/0117125, the disclosures of which are herein incorporated in their entirety.

The terms “cationic lipid” and “amino lipid” are used interchangeably herein to include those lipids and salts thereof having one, two, three, or more fatty acid or fatty alkyl chains and a pH-titratable amino group (e.g., an alkylamino or dialkylamino group). The cationic lipid is typically protonated (i.e., positively charged) at a pH below the pKa of the cationic lipid and is substantially neutral at a pH above the pKa. The cationic lipids can also be titratable cationic lipids. In certain embodiments, the cationic lipids comprise: a protonatable tertiary amine (e.g., pH-titratable) group; C18 alkyl chains, wherein each alkyl chain independently has 0 to 3 (e.g., 0, 1, 2, or 3) double bonds; and ether, ester, or ketal linkages between the head group and alkyl chains.

Cationic lipids can include, without limitation, 1,2-dilinoleyloxy-N,N-dimethylaminopropane (DLinDMA), 1,2-dilinolenyloxy-N,N-dimethylaminopropane (DLenDMA), 1,2-di-y-linolenyloxy-N,N-dimethylaminopropane (g-DLenDMA), 2,2-dilinoleyl-4-(2-dimethylaminoethyl)-[1,3]-dioxolane (DLin-K-C2-DMA, also known as DLin-C2K-DMA, XTC2, and C2K), 2,2-dilinoleyl-4-dimethylaminomethyl-[1,3]-dioxolane (DLin-K-DMA), dilinoleylmethyl-3-dimethylaminopropionate (DLin-M-C2-DMA, also known as MC2), (6Z,9Z,28Z,31 Z)-heptatriaconta-6,9,28,31-tetraen-19-yl 4-(dimethylamino)butanoate (DLin-M-C3-DMA, also known as MC3), salts thereof, and mixtures thereof. Other cationic lipids also include, but are not limited to 1,2-distearyloxy-N,N-dimethyl-3-aminopropane (DSDMA), 1,2-dioleyloxy-N,N-dimethyl-3-aminopropane (DODMA), 2,2-dilinoleyl-4-(3-dimethylaminopropyl)-[1,3]-dioxolane (DLin-K-C3-DMA), 2,2-dilinoleyl-4-(3-dimethylaminobutyl)-[1,3]-dioxolane (DLin-K-C4-DMA), DLen-C2K-DMA, y-DLen-C2K-DMA, and (DLin-MP-DMA) (also known as 1-B11).

Still further cationic lipids can include, without limitation, 2,2-dilinoleyl-5-dimethylaminomethyl-[1,3]-dioxane (DLin-K6-DMA), 2,2-dilinoleyl-4-N-methylpepiazino-[1,3]-dioxolane (DLin-K-MPZ), 1,2-dilinoleylcarbamoyloxy-3-dimethylaminopropane (DLin-C-DAP), 1,2-dilinoleyoxy-3-(dimethylamino)acetoxypropane (DLin-DAC), 1,2-dilinoleyoxy-3-morpholinopropane (DLin-MA), 1,2-dilinoleoyl-3-dimethylaminopropane (DLinDAP), 1,2-dilinoleylthio-3-dimethylaminopropane (DLin-S-DMA), 1-linoleoyl-2-linoleyloxy-3-dimethylaminopropane (DLin-2-DMAP), 1,2-dilinoleyloxy-3-trimethylaminopropane chloride salt (DLin-TMA.C1), 1,2-dilinoleoyl-3-trimethylaminopropane chloride salt (DLin-TAP.C1), 1,2-dilinoleyloxy-3-(N-methylpiperazino)propane (DLin-MPZ), 3-(N,N-dilinoleylamino)-1, 2-propanediol (DLinAP), 3-(N,N-dioleylamino)-1,2-propanedio (DOAP), 1,2-dilinoleyloxo-3-(2-N,N-dimethylamino)ethoxypropane (DLin-EG-DMA), N,N-dioleyl-N,N-dimethylammonium chloride (DODAC), N-(1-(2,3-dioleyloxy)propyl)-N,N,N-trimethylammonium chloride (DOTMA), N,N-distearyl-N,N-dimethylammonium bromide (DDAB), N-(1-(2,3-dioleoyloxy)propyl)-N,N,N-trimethylammonium chloride (DOTAP), 3-(N—(N′,N′-dimethylaminoethane)-carbamoyl)cholesterol (DC-Chol), N-(1,2-dimyristyloxyprop-3-yl)-N,N-dimethyl-N-hydroxyethyl ammonium bromide (DMRIE), 2,3-dioleyloxy-N-[2(spermine-carboxamido)ethyl]-N,N-dimethyl-1-propanaminiumtrifluoroacetate (DOSPA), dioctadecylamidoglycyl spermine (DOGS), 3-dimethylamino-2-(cholest-5-en-3-beta-oxybutan-4-oxy)-1-(cis,cis-9,12-octadecadienoxy)propane (CLinDMA), 2-[5′-(cholest-5-en-3-beta-oxy)-3′-oxapentoxy)-3-dimethyl-1-(cis,cis-9′,1-2′-octadecadienoxy)propane (CpLinDMA), N,N-dimethyl-3,4-dioleyloxybenzylamine (DMOBA), 1,2-N,N′-dioleylcarbamyl-3-dimethylaminopropane (DOcarbDAP), 1,2-N,N′-dilinoleylcarbamyl-3-dimethylaminopropane (DLincarbDAP), dexamethasone-sperimine (DS) and disubstituted spermine (D2S) or mixtures thereof.

A number of commercial preparations of cationic lipids can be used, such as, LIPOFECTIN® (including DOTMA and DOPE, available from GIBCO/BRL), and LIPOFECT AMINE® (comprising DOSPA and DOPE, available from GIBCO/BRL).

In certain embodiments, cationic lipid can be present in an amount from about 10% by weight of the LNP to about 85% by weight of the lipid nanoparticle, or from about 50% by weight of the LNP to about 75% by weight of the LNP.

Sterols can confer fluidity to the LNP. As used herein, “sterol” refers to any naturally occurring sterol of plant (phytosterols) or animal (zoosterols) origin as well as non-naturally occurring synthetic sterols, all of which are characterized by the presence of a hydroxyl group at the 3-position of the steroid A-ring. The sterol can be any sterol conventionally used in the field of liposome, lipid vesicle or lipid particle preparation, most commonly cholesterol. Phytosterols can include campesterol, sitosterol, and stigmasterol. Sterols also include sterol-modified lipids, such as those described in U.S. Patent Application Publication 2011/0177156, the disclosure of which is herein incorporated in its entirety. In certain embodiments, a sterol can be present in an amount from about 5% by weight of the LNP to about 50% by weight of the lipid nanoparticle or from about 10% by weight of the LNP to about 25% by weight of the LNP.

LNP can comprise a neutral lipid. Neutral lipids can comprise any lipid species which exists either in an uncharged or neutral zwitterionic form at physiological pH. Such lipids include, without limitation, diacylphosphatidylcholine, diacylphosphatidylethanolamine, ceramide, sphingomyelin, dihydrosphingomyelin, cephalin, and cerebrosides. The selection of neutral lipids is generally guided by consideration of, inter alia, particle size and the requisite stability. In certain embodiments, the neutral lipid component can be a lipid having two acyl groups (e.g., diacylphosphatidylcholine and diacylphosphatidylethanolamine).

Lipids having a variety of acyl chain groups of varying chain length and degree of saturation are available or can be isolated or synthesized by well-known techniques. In certain embodiments, lipids containing saturated fatty acids with carbon chain lengths in the range of C14 to C22 can be used. In another group of embodiments, lipids with mono or diunsaturated fatty acids with carbon chain lengths in the range of C14 to C22 are used. Additionally, lipids having mixtures of saturated and unsaturated fatty acid chains can be used. Exemplary neutral lipids include, without limitation, 1,2-dioleoyl-sn-glycero-3-phosphatidyl-ethanolamine (DOPE), 1,2-distearoyl-sn-glycero-3-phosphocholine (DSPC), 1-palmitoyl-2-oleoyl-sn-glycero-3-phosphocholine (POPC), or any related phosphatidylcholine. The neutral lipids can also be composed of sphingomyelin, dihydrosphingomyelin, or phospholipids with other head groups, such as serine and inositol.

In certain embodiments, the neutral lipid can be present in an amount from about 0.1% by weight of the lipid nanoparticle to about 75% by weight of the LNP, or from about 5% by weight of the LNP to about 15% by weight of the LNP.

LNP encapsulated nucleic acids, expression cassettes, AAV vectors, and non-viral vectors can be incorporated into pharmaceutical compositions, e.g., a pharmaceutically acceptable carrier or excipient. Such pharmaceutical compositions are useful for, among other things, administration and delivery of LNP encapsulated nucleic acids, expression cassettes, AAV vectors, and non-viral vectors to a subject in vivo or ex vivo.

Preparations of LNP can be combined with additional components. Non-limiting examples include polyethylene glycol (PEG) and sterols.

The term “PEG” refers to a polyethylene glycol, a linear, water-soluble polymer of ethylene PEG repeating units with two terminal hydroxyl groups. PEGs are classified by their molecular weights; for example, PEG 2000 has an average molecular weight of about 2,000 daltons, and PEG 5000 has an average molecular weight of about 5,000 daltons. PEGs are commercially available from Sigma Chemical Co. and other companies and include, for example, the following functional PEGs: monomethoxypolyethylene glycol (MePEG-OH), monomethoxypolyethylene glycol-succinate (MePEG-S), monomethoxypolyethylene glycol-succinimidyl succinate (MePEG-S-NHS), monomethoxypolyethylene glycol-amine (MePEG-NH2), monomethoxypolyethylene glycol-tresylate (MePEG-TRES), and monomethoxypolyethylene glycol-imidazolyl-carbonyl (MePEG-IM).

In certain embodiments, PEG can be a polyethylene glycol with an average molecular weight of about 550 to about 10,000 daltons and is optionally substituted by alkyl, alkoxy, acyl or aryl. In certain embodiments, the PEG can be substituted with methyl at the terminal hydroxyl position. In certain embodiments, the PEG can have an average molecular weight from about 750 to about 5,000 daltons, or from about 1,000 to about 5,000 daltons, or from about 1,500 to about 3,000 daltons or from about 2,000 daltons or of about 750 daltons. The PEG can be optionally substituted with alkyl, alkoxy, acyl or aryl. In certain embodiments, the terminal hydroxyl group can be substituted with a methoxy or methyl group.

PEG-modified lipids include the PEG-dialkyloxypropyl conjugates (PEG-DAA) described in U.S. Pat. Nos. 8,936,942 and 7,803,397, the disclosures of which are herein incorporated in their entirety. PEG-modified lipids (or lipid-polyoxyethylene conjugates) that are useful can have a variety of “anchoring” lipid portions to secure the PEG portion to the surface of the lipid vesicle. Examples of suitable PEG-modified lipids include PEG-modified phosphatidylethanolamine and phosphatidic acid, PEG-ceramide conjugates (e.g., PEG-CerC14 or PEG-CerC20) which are described in U.S. Pat. No. 5,820,873, the disclosure of which is herein incorporated in its entirety, PEG-modified dialkylamines and PEG-modified 1,2-diacyloxypropan-3-amines. In certain embodiments, the PEG-modified lipid can be PEG-modified diacylglycerols and dialkylglycerols. In certain embodiments, the PEG can be in an amount from about 0.5% by weight of the LNP to about 20% by weight of the LNP, or from about 5% by weight of the LNP to about 15% by weight of the LNP.

Furthermore, LNP can be a PEG-modified and a sterol-modified LNP. The LNPs, combined with additional components, can be the same or separate LNPs. In other words, the same LNP can be PEG modified and sterol modified or, alternatively, a first LNP can be PEG modified and a second LNP can be sterol modified. Optionally, the first and second modified LNPs can be combined.

In certain embodiments, prior to encapsulating LNPs can have a size in a range from about 10 nm to 500 nm, or from about 50 nm to about 200 nm, or from 75 nm to about 125 nm. In certain embodiments, LNP encapsulated nucleic acid, expression vector, AAV vector, or non-viral vector can have a size in a range from about 10 nm to 500 nm.

### Polymer-Based Systems

Polymer-based delivery systems are well known in the art, and any suitable polymer-based delivery system or polymeric nanoparticle known to those skilled in the art in view of the present disclosure can be used in the invention. DNA can be entrapped into the polymeric matrix of polymeric nanoparticles or can be adsorbed or conjugated on the surface of the nanoparticles. Examples of commonly used polymers for gene delivery include, e.g., poly(lactic-co-glycolic acid) (PLGA), poly lactic acid (PLA), poly(ethylene imine) (PEI), chitosan, dendrimers, polyanhydride, polycaprolactone, and polymethacrylates.

The polymeric-based non-viral vectors can have different sizes, ranging from about 1 nm to about 1000 nm, optionally from about 10 nm to about 500 nm, optionally from about 50 nm to about 200 nm, optionally about 100 nm to about 150 nm, optionally about 150 nm or less.

### Protein-Based Systems

Protein-based delivery systems are well known in the art, and any suitable protein-based delivery system or cell-penetrating peptide (CPP) known to those skilled in the art in view of the present disclosure can be used in the invention.

CPPs are short peptides (6-30 amino acid residues) that are potentially capable of intracellular penetration to deliver therapeutic molecules. The majority of CPPs consists mainly of arginine and lysine residues, making them cationic and hydrophilic, but CPPs can also be amphiphilic, anionic, or hydrophobic. CPPs can be derived from natural biomolecules (e.g., Tat, an HIV-1 protein), or obtained by synthetic methods (e.g., poly-L-lysine, polyarginine) (Singh et al., Drug Deliv. 2018; 25(1):1996-2006). Examples of CPPs include, e.g., cationic CPPs (highly positively charged) (e.g., the Tat peptide, penetratin, protamine, poly-L-lysine, polyarginine, etc.); amphipathic CPPs (chimeric or fused peptides, constructed from different sources, containing both positively and negatively charged amino acid sequences) (e.g., transportan, VT5, bactenecin-7 (Bac7), proline-rich peptide (PPR), SAP (VRLPPP)₃, TP10, pep-1, MPG, etc.); membranotropic CPPs (exhibit both hydrophobic and amphipathic nature simultaneously, and comprise both large aromatic residues and small residues) (e.g., gH625, SPIONs-PEG-CPP NPs, etc.); and hydrophobic CPPs (contain only non-polar motifs or residues) (e.g., SG3, PFVYLI, pep-7, fibroblast growth factors (FGF), etc.).

The protein-based non-viral vectors can have different sizes, ranging from about 1 nm to about 1000 nm, optionally from about 10 nm to about 500 nm, optionally from about 50 nm to about 200 nm, optionally about 100 nm to about 150 nm, optionally about 150 nm or less.

### Peptide Cage Systems

Peptide cage-based delivery systems are well known in the art, and any suitable peptide cage-based delivery system known to those skilled in the art in view of the present disclosure can be used in the invention. In general, any proteinaceous material that is able to be assembled into a cage-like structure, forming a constrained internal environment, can be used. Several different types of protein “shells” can be assembled and loaded with different types of materials. For example, protein cages comprising a shell of viral coat protein(s) (e.g., from the Cowpea Chlorotic Mottle Virus (CCMV) protein coat) that encapsulate a non-viral material, as well as protein cages formed from non-viral proteins have been described (see, e.g., U.S. Pat. Nos. 6,180,389 and 6,984,386, U.S. Patent Application 20040028694, and U.S. Patent Application 20090035389, the disclosures of which are herein incorporated in their entirety). Peptide cages can comprise a proteinaceous shell that self-assembles to form a protein cage (e.g., a structure with an interior cavity which is either naturally accessible to the solvent or can be made to be so by altering solvent concentration, pH, equilibria ratios).

Examples of protein cages derived from non-viral proteins include, e.g., ferritins and apoferritins, derived from both eukaryotic and prokaryotic species, e.g., 12 and 24 subunit ferritins; and protein cages formed from heat shock proteins (HSPs), e.g., the class of 24 subunit heat shock proteins that form an internal core space, the small HSP of Methanococcus jannaschii, the dodecameric Dsp HSP of E. coli, the MrgA protein, etc. As will be appreciated by those in the art, the monomers of the protein cages can be naturally occurring or variant forms, including amino acid substitutions, insertions and deletions (e.g., fragments) that can be made.

The protein cages can have different core sizes, ranging from about 1 nm to about 1000 nm, optionally from about 10 nm to about 500 nm, optionally from about 50 nm to about 200 nm, optionally about 100 nm to about 150 nm, optionally about 150 nm or less.

### Administration and Treatment

The instant invention may be used in human and veterinary medical applications. Suitable subjects therefore include mammals, such as humans, as well as non-human mammals. The term “subject” refers to an animal, typically a mammal, such as humans, non-human primates (apes, gibbons, gorillas, chimpanzees, orangutans, macaques), a domestic animal (dogs and cats), a farm animal (poultry such as chickens and ducks, horses, cows, goats, sheep, pigs), and experimental animals (mouse, rat, rabbit, guinea pig). Human subjects include fetal, neonatal, infant, juvenile and adult subjects.

As used herein, the terms “administering”, or “administration” refers to providing one or more compositions described herein to a patient or a subject. By way of example and without limitation, composition administration, e.g., injection, can be performed by intravenous (i.v.) injection, sub-cutaneous (s.c.) injection, intradermal (i.d.) injection, intraperitoneal (i.p.) injection, or intramuscular (i.m.) injection. One or more such routes can be employed. Parenteral administration can be, for example, by bolus injection or by gradual perfusion over time. Alternatively, or concurrently, administration can be by the oral route.

The terms “treatment,” “treat,” and “treating” refer to a clinical intervention aimed to reverse, alleviate, delay the onset of, or inhibit the progress of a disease or disorder, or one or more symptoms thereof, as described herein. As used herein, the terms “treatment,” “treat,” and “treating” refer to a clinical intervention aimed to reverse, alleviate, delay the onset of, or inhibit the progress of a disease or disorder, or one or more symptoms thereof, as described herein. In some embodiments, treatment may be administered after one or more symptoms have developed and/or after a disease has been diagnosed. In other embodiments, treatment may be administered in the absence of symptoms, e.g., to prevent or delay onset of a symptom or inhibit onset or progression of a disease. For example, treatment may be administered to a susceptible individual prior to the onset of symptoms (e.g., in light of a history of symptoms and/or in light of genetic or other susceptibility factors). Treatment may also be continued after symptoms have resolved, for example, to prevent or delay their recurrence.

The doses of an “effective amount” or “sufficient amount” for treatment (e.g., to ameliorate or to provide a therapeutic benefit or improvement) typically are effective to provide a response to one, multiple or all adverse symptoms, consequences or complications of the disease, one or more adverse symptoms, disorders, illnesses, pathologies, or complications, for example, caused by or associated with the disease, to a measurable extent, although decreasing, reducing, inhibiting, suppressing, limiting or controlling progression or worsening of the disease is a satisfactory outcome.

An effective amount or a sufficient amount can but need not be provided in a single administration, can require multiple administrations, and, can but need not be, administered alone or in combination with another composition (e.g., agent), treatment, protocol or therapeutic regimen. For example, the amount can be proportionally increased as indicated by the need of the subject, type, status and severity of the disease treated or side effects (if any) of treatment. In addition, an effective amount or a sufficient amount need not be effective or sufficient if given in single or multiple doses without a second composition (e.g., another drug or agent), treatment, protocol or therapeutic regimen, since additional doses, amounts or duration above and beyond such doses, or additional compositions (e.g., drugs or agents), treatments, protocols or therapeutic regimens can be included in order to be considered effective or sufficient in a given subject.

An effective amount or a sufficient amount need not be effective in each and every subject treated, nor a majority of treated subjects in a given group or population. An effective amount or a sufficient amount means effectiveness or sufficiency in a particular subject, not a group or the general population. As is typical for such methods, some subjects will exhibit a greater response, or less or no response to a given treatment method or use.

As used herein, the terms and phrases “co-delivery,” and “administered together with” in the context of the administration of two or more therapies or components to a subject refers to simultaneous administration of two or more therapies or components. “Simultaneous administration” can be administration of the two components at least within the same day. When two components are “administered together with” or “administered in combination with,” they can be administered in separate compositions sequentially within a short time period, such as 24, 20, 16, 12, 8 or 4 hours, or within 1 hour, or within 30 minutes, or within 10 minutes, or within 5 minutes, or within 2 minutes, or they can be administered in a single composition at the same time.

The order and timing of administration of one or more gene editing agents and an episomal vector can vary, depending on the type and severity of the disease being treated. For example, an episomal vector can be administered prior to (e.g., 5 minutes, 15 minutes, 30 minutes, 45 minutes, 1 hour, 2 hours, 4 hours, 6 hours, 12 hours, 16 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 16 hours, 24 hours, 48 hours, 72 hours, 96 hours, 1 week, 2 weeks, 3 weeks, 4 weeks, 5 weeks, 6 weeks, 8 weeks, or 12 weeks after) the administration of one or more gene editing agents.

Treatment doses of episomal vector can vary and depend upon the type, onset, progression, severity, frequency, duration, or probability of the disease or disorder to which treatment is directed, the clinical endpoint desired, previous or simultaneous treatments, the general health, age, gender, race or immunological competency of the subject and other factors that will be appreciated by the skilled artisan. The dose amount, number, frequency, or duration can be proportionally increased or reduced, as indicated by any adverse side effects, complications or other risk factors of the treatment or therapy and the status of the subject.

The dose to achieve a therapeutic effect, e.g., episomal vector dose in mg per kilogram of body weight (mg/kg), will also vary based on several factors including route of administration, the level of gene expression required to achieve a therapeutic effect, the specific disease or disorder treated, host immune response to DNA, host immune response transgene expression product, and the stability of the protein, peptide, or nucleic acid expressed. Based on the guidance provided herein, one skilled in the art can determine a suitable episomal vector dose range to treat a patient having a particular disease or disorder.

The overall level of gene expression can vary depending upon the use of the episomal vector. In different embodiments of gene therapy providing a therapeutic protein, the provided expression or activity is at least 1%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100% of normal expression of the corresponding subject protein.

The dose of editing agent to achieve regulation of expression, e.g., dose in mg per kilogram of body weight (mg/kg), will also vary based on several factors including the route of administration specific disease or disorder treated, whether the editing agent is targeting DNA or RNA, whether gene or mRNA expression is being increased or decreased, or stability of the guide RNA. Based on the guidance provided herein, one skilled in the art can determine a suitable dose range of the editing agent to treat a patient having a particular disease or disorder.

In certain embodiment, varying amounts of the targeting ribonucleic acid are administered to the subject to obtain varying expression levels of the gene. In certain embodiments, varying amounts of the base editor are administered to obtain varying expression levels of the gene. In certain embodiments, varying amounts for the targeting ribonucleic acid and base editor are administered to obtain varying expression levels of the gene.

Regulation of expression by one or more editing agents can occur within 1 month of administering one or more nucleic acid molecules encoding one or more gene editing agents, for example, within 4 weeks, 3 weeks, 2 weeks, 1 week, 6 days, 5 days, 4 days, 72 hours, 48 hours, 24 hours, 12 hours, 8 hours, 4 hours, 2 hours of administering one or more nucleic acid molecules encoding one or more editing agents.

### Illustrative Disease and Disorder

Diseases and disorders that can be treated include lung disease (e.g., cystic fibrosis), a blood disorder (e.g., anemia), CNS diseases and disorder, epilepsy, a lysosomal storage disease (e.g., aspartylglucosaminuria), Rett syndrome, Batten disease, late infantile neuronal ceroid lipofuscinosis type 2 (CLN2), cystinosis, Fabry disease, Gaucher disease types I, II, and III, glycogen storage disease II (Pompe disease), GM2-gangliosidosis type I (Tay-Sachs disease), GM2-gangliosidosis type II (Sandhoff disease), mucolipidosis types I (sialidosis type I and II), II (I-cell disease), III (pseudo-Hurler disease) and IV, mucopolysaccharide storage diseases (Hurler disease and variants, Hunter, Sanfilippo Types A,B,C,D, Morquio Types A and B, Maroteaux-Lamy and Sly diseases), Niemann-Pick disease types A/B, C1 and C2, and Schindler disease types I and II), hereditary angioedema (HAE), a copper or iron accumulation disorder (e.g., Wilson's or Menkes disease), lysosomal acid lipase deficiency, a neurological or neurodegenerative disorder, cancer, type 1 or type 2 diabetes, adenosine deaminase deficiency, a metabolic defect (e.g., glycogen storage diseases), and a disease of solid organs (e.g., brain, liver, kidney, heart).

Synucleinopathies can be treated by methods according to the instant invention. Synucleinopathies are neurodegenerative diseases or disorders characterized by neuronal and/or glial inclusions. Pathologically, synucleinopathies can be divided into two major disease groups: Lewy body diseases or disorders and multiple system atrophy (MSA). Lewy body diseases and disorders are characterized by aggregated α-synuclein, and include Parkinson's disease, Parkinson's disease dementia, dementia with Lewy bodies, infantile neuroaxonal dystrophy, atypical neuroaxonal dystrophy, adult-onset dystonia-parkinsonism, autosomal recessive early-onset parkinsonism, POLG-associated neurodegeneration, Niemann-Pick type C1, and Krabbe disease. (Koga et al. Molecular Neurodegeneration (2021) 16:83).

Parkinson's disease can be treated by methods according to the instant invention. Parkinson's disease is an age-related progressive neurodegenerative disorder. Parkinson's disease is characterized by the abnormal accumulation of misfolded α-synuclein protein aggregates in various regions of the brain. Dopaminergic neuronal loss in the substantia nigra is a pathologic hallmark of Parkinson's disease. (Lee et al. Neuroimmunol. Neuroinflammation (2021) 8:222-44; and Koga et al. Molecular Neurodegeneration (2021) 16:83.)

Glycogen storage disease type II, also called Pompe disease can be treated by methods according to the instant invention. Pompe disease is an autosomal recessive disorder caused by mutations in the gene encoding the lysosomal enzyme acid α-glucosidase (GAA), which catalyzes the degradation of glycogen. The resulting enzyme deficiency leads to pathological accumulation of glycogen and lysosomal alterations in body tissues, resulting in cardiac, respiratory, and skeletal muscle dysfunction.

Blood clotting disorders which can be treated include hemophilia A, hemophilia A with inhibitory antibodies, hemophilia B, hemophilia B with inhibitory antibodies, a deficiency in any coagulation Factor: VII, VIII, IX, X, XI, V, XII, II, von Willebrand factor, or a combined FV/FVIII deficiency, thalassemia, vitamin K epoxide reductase C1 deficiency or gamma-carboxylase deficiency.

Other diseases and disorders that can be treated include bleeding associated with trauma, injury, thrombosis, thrombocytopenia, stroke, coagulopathy, disseminated intravascular coagulation (DIC); over-anticoagulation associated with heparin, low molecular weight heparin, pentasaccharide, warfarin, small molecule antithrombotics (i.e., FXa inhibitors), or a platelet disorder such as, Bernard Soulier syndrome, Glanzmann thrombasthenia, or storage pool deficiency.

Other diseases and disorders that can be treated include proliferative diseases (e.g., cancers, tumors and dysplasias), Crigler-Najjar and metabolic diseases like metabolic diseases of the liver; Friedreich ataxia; infectious diseases; viral diseases induced for example by hepatitis B or C viruses, HIV, herpes, and retroviruses; genetic diseases such as cystic fibrosis, dystroglycanopathies, myopathies such as Duchenne muscular myopathy or dystrophy, myotubular myopathy, sickle-cell anemia, sickle cell disease, Fanconi's anemia, diabetes, amyotrophic lateral sclerosis (ALS), myotubularin myopathy, motor neuron diseases such as spinal muscular atrophy (SMA), spinobulbar muscular atrophy, or Charcot-Marie-Tooth disease; arthritis; severe combined immunodeficiencies such as RS-SCID, ADA-SCID or X-SCID; Wiskott-Aldrich syndrome; X-linked thrombocytopenia; X-linked congenital neutropenia; chronic granulomatous disease; clotting factor deficiencies; cardiovascular disease such as restenosis, ischemia, dyslipidemia, and homozygous familial hypercholesterolemia; eye or ocular diseases such as retinitis pigmentosa, X-linked retinitis pigmentosa, autosomal dominant retinitis pigmentosa, recessive retinitis pigmentosa, choroideremia, choroidal neovascularization, gyrate atrophy, retinoschisis, X-linked retinoschisis, macular degeneration, diabetic macular edema (DME), diabetic retinopathy associated with DME, wet age-related macular degeneration (wet AMD or wAMD), macular edema following retinal vein occlusion, non-arteritic ischaemic optic neuropathy, Leber congenital amaurosis, Leber hereditary optic neuropathy, achromatopsia, and Stargardt disease; lysosomal storage diseases such as San Filippo syndrome; hyperbilirubinemia such as CN type I or II or Gilbert's syndrome; glycogen storage disease such as GSDI, GSDII (Pompe disease), GSDIII, GSDIV, GSDV, GSDVI, GSDVII, GSDVIII or lethal congenital glycogen storage disease of the heart.

In certain embodiments, the subject has a disease or disorder that affects or originates in the central nervous system (CNS). In certain embodiments, the disease is a neurodegenerative disease. Non-limiting examples of CNS or neurodegenerative disease include Alzheimer's disease, Huntington's disease, ALS, hereditary spastic hemiplegia, primary lateral sclerosis, spinal muscular atrophy, Kennedy's disease, a polyglutamine repeat disease, or Parkinson's disease. In certain embodiments, the disease is a psychiatric disease, an addiction (e.g., to tobacco, alcohol, or drugs), epilepsy, Canavan's disease, or adrenoleukodystrophy. In certain embodiments, the CNS or neurodegenerative disease is a polyglutamine repeat disease such as, spinocerebellar ataxia (SCA1, SCA2, SCA3, SCA6, SCA7, or SCA17).

In certain embodiments, the subject has a disease or disorder related to pain, such as chronic pain or neuropathic pain. Gene therapy targets are described in Ovespian and Waxman, Nat Rev Neurosci. 2023 April; 24(4):252-265, the content of which is incorporated by referenced herein in its entirety.

A number of different aspect and embodiments of the instant invention have been described throughout the application. Nevertheless, the skilled artisan without departing from the spirit and scope of the instant invention, can make various changes and modifications of the instant invention to adapt it to various usages and conditions.

### EMBODIMENTS

### Embodiments A

1. A method of regulating expression of a gene located on an episomal vector in a subject in need thereof, comprising administering to the subject one or more gene editing agents that modify a region of the gene to thereby regulate the expression of the gene, wherein the region of the gene comprises one or more of a promoter, an enhancer, a silencer, or an insulator, a premature termination codon convertible to an amino acid codon via the modification of the region, or an amino acid codon convertible to a premature termination codon via the modification of the region.

2. The method of embodiment 1, wherein the one or more gene editing agents comprise a guide RNA that is complementary to the region of the gene and a Cas protein or a derivative of the Cas protein.

3. The method of embodiment 2, wherein the Cas protein is Cas9, such as Staphylococcus aureus Cas9 (SaCas9), Streptococcus thermophilus 1 Cas9 (St1Cas9), a modified Streptococcus pyogenes Cas9 (SpCas9); a CpF1; a CasX; a CasY; a C2c1; a C2c2; a C2c3; or a variant thereof.

4. The method of any one of the foregoing embodiments, wherein the one or more gene editing agents further comprise a donor nucleic acid having at least one nucleotide change relative to the region of the gene and capable of integrating into the region of the gene to modify the region.

5. The method of any of the foregoing embodiments, wherein the one or more gene editing agents are encoded by one or more nucleic acid molecules administered to the subject, preferably the one or more gene editing agents are encoded by a RNA molecule, particularly an mRNA molecule, administered to the subject.

6. The method of embodiment 5, wherein the one or more nucleic acid molecules, such as the one or more mRNA molecules, are administered to the subject with a liposome, a lipid nanoparticle (LNP), a peptide cage, or a polymer nanoparticle.

7. A method of regulating expression of a gene located on an episomal vector in a subject in need thereof, comprising administering to the subject a base editor system that effects a base alteration in a region of the gene or a region of an mRNA transcript of the gene to thereby regulate the expression of the gene.

8. The method of embodiment 7, wherein the region of the gene comprises one or more of a promoter, an enhancer, a silencer or an insulator, or the region of the gene or the region of the mRNA transcript comprises a premature termination codon convertible to an amino acid codon via the base alteration, or an amino acid codon convertible to a premature termination codon via the base alteration.

9. The method of embodiment 7 or 8, wherein the base editor system comprises: a ribonucleic acid complementary to the region of the gene; and a base editor comprising a polynucleotide programmable DNA binding domain and an adenosine deaminase domain or a cytidine deaminase domain, wherein the polynucleotide programmable DNA binding domain in conjunction with the ribonucleic acid binds to the region of the gene to effect the base alteration.

10. The method of embodiment 9, wherein the polynucleotide programmable DNA binding domain comprises a nuclease inactive variant of a Cas protein or a nickase variant of a Cas protein.

11. The method of embodiment 10, wherein the Cas protein is Cas9, such as Staphylococcus aureus Cas9 (SaCas9), Streptococcus thermophilus 1 Cas9 (St1Cas9), a modified Streptococcus pyogenes Cas9 (SpCas9); a CpF1; a CasX; a CasY; a C2c1; a C2c2; a C2c3; or a variant thereof.

12. The method of any one of embodiments 9-11, wherein the base editor further comprises a uracil binding protein, such as a uracil glycosylase inhibitor (UGI) domain that inhibits a uracil-DNA glycosylase.

13. The method of any one of embodiments 9-12, wherein (i) the cytidine deaminase domain is selected from the group consisting of the apolipoprotein B mRNA-editing enzyme, catalytic polypeptide-like (APOBEC) family of deaminases, such as APOBEC1, APOBEC2, APOBEC3A, APOBEC3B, APOBEC3C, APOBEC3D/E, APOBEC3F, APOBEC3G, APOBEC3H, or APOBEC4; activation-induced cytidine deaminase (AID), such as activation induced cytidine deaminase (AICDA); cytosine deaminase 1 (CDA1) or CDA2; or cytosine deaminase acting on tRNA (CDAT), and (ii) the adenosine deaminase is selected from the group consisting of adenosine deaminase 1 (ADA1) and ADA2.

14. The method of embodiment 7 or 8, wherein the base editor system comprises: a ribonucleic acid complementary to the region of the mRNA transcript; and a base editor comprising a polynucleotide programmable RNA binding domain and an adenosine deaminase domain or a cytidine deaminase domain, wherein the polynucleotide programmable RNA binding domain in conjunction with the ribonucleic acid binds to the region of the mRNA transcript to effect the base alteration.

15. The method of embodiment 14, wherein the polynucleotide programmable RNA binding domain comprises a nuclease inactive variant of Cas13 or a nickase variant of Cas13.

16. The method of embodiment 15, wherein the Cas13 is Cas13a and Cas13b.

17. The method of any one of embodiments 14-16, wherein (i) the cytidine deaminase domain is selected from the group consisting of the apolipoprotein B mRNA-editing enzyme, catalytic polypeptide-like (APOBEC) family of deaminases, such as APOBEC1, APOBEC2, APOBEC3A, APOBEC3B, APOBEC3C, APOBEC3D/E, APOBEC3F, APOBEC3G, APOBEC3H, or APOBEC4; activation-induced cytidine deaminase (AID), such as activation induced cytidine deaminase (AICDA); cytosine deaminase 1 (CDA1) or CDA2; or cytosine deaminase acting on tRNA (CDAT), and (ii) the adenosine deaminase is selected from the group consisting of adenosine deaminase acting on RNA 1 (ADAR1), ADAR2, ADAR3; adenosine deaminase acting on tRNA 1 (ADAT1), ADAT2, ADAT3; and naturally occurring or engineered tRNA-specific adenosine deaminase (TadA).

18. The method of any one of embodiments 9-17, wherein the ribonucleic acid is a guide RNA.

19. The method of any of embodiments 7-18, wherein the base editor system or a component thereof is encoded by one or more nucleic acid molecules administered to the subject, preferably the ribonucleic acid and the base editor are encoded by one or more RNA molecules, such as one or more mRNA molecules, administered to the subject.

20. The method of embodiment 19, wherein the one or more nucleic acid molecules, such as the one or more mRNA molecules, are administered to the subject with a lipid nanoparticle (LNP), a peptide cage, or a polymer nanoparticle.

21. The method of any one of embodiments 7-20, wherein the base alteration results in a conversion of the amino acid codon to the premature stop codon, preferably upstream of a splice junction, to thereby down-regulate the expression of the gene.

22. The method of embodiment 21, wherein the base alteration results in the conversion of a CGA, CAG, or TGG codon to a premature TGA, TAG, or TAA stop codon, respectively, and the base editor comprises the cytidine deaminase domain, preferably, the CAG codon is located in proximity to the 5′-end of the gene.

23. The method of any one of embodiments 7-20, wherein the base alteration results in a conversion of a premature stop codon to an amino acid codon to thereby up-regulate the expression of the gene.

24. The method of embodiment 23, wherein the base alteration results in the conversion of a premature UAG, UAA, or UGA stop codon to a CAG, CAA, or CGA, respectively, and the base editor comprises the adenosine deaminase domain, preferably, the premature UAG stop codon is located in proximity to the 5′-end of the gene.

25. The method of any one of the foregoing claims, further comprising administering to the subject the episomal vector comprising the gene.

26. The method of any one of the foregoing claims, wherein the episomal vector is a nonviral vector, such as a plasmid, or a viral vector, such as an adeno-associated viral (AAV) vector, a lentivirus vector, or an adenovirus vector.

27. The method of embodiment 26, wherein the episomal vector is an AAV vector.

28. The method of any one of the foregoing, wherein the subject is a human, such as a human subject suffering from a disease selected from the group consisting of Hereditary angioedema, Pompe disease, hemophilia A, hemophilia B, Fabry disease, Huntington disease, Parkinson's disease, Alzheimer's disease, a synucleinopathy, epilepsy, neuropathic pain, wet macular degeneration, Usher 1F, Usher 1B, glaucoma, Leber congenital amaurosis, and Stargardt disease.

### Embodiments B

1. A method of regulating expression of a gene located on an episomal vector in a subject in need thereof, comprising administering to the subject an editing agent that effects an alteration in a region of an mRNA transcript of the gene to thereby regulate the expression of the gene.

2. The method of embodiment 1, wherein the editing agent effects a base alteration in the region of the mRNA transcript of the gene.

3. The method of embodiment 1, wherein the alteration in a region of an mRNA transcript of the gene alters the stability of the mRNA transcript, the initiation or level of the translation of the mRNA transcript, the stability and/or activity of the translated protein.

4. The method of embodiment 2, wherein the region of the mRNA transcript comprises a premature termination codon convertible to an amino acid codon via the base alteration, or an amino acid codon convertible to a premature termination codon via the base alteration.

5. The method of embodiment 2, wherein the base alteration (a) is within a microRNA targeting site or a toe-hold switch site, or (b) induces a ribosomal frameshift or alters a codon encoding an amino acid residue critical to function and/or structure of an encoded protein.

6. The method of any one of embodiments 2-5, wherein the editing agent comprises a targeting ribonucleic acid complementary to the region of the mRNA transcript.

7. The method of embodiment 6, wherein the targeting ribonucleic acid is linear.

8. The method of embodiment 6, wherein the targeting ribonucleic acid is circular.

9. The method of any one of embodiments 6-8, wherein the targeting ribonucleic acid effects the base alteration via binding to an endogenous adenosine deaminase domain.

10. The method of embodiment 9, wherein the adenosine deaminase is selected from the group consisting of adenosine deaminase acting on RNA 1 (ADAR1), ADAR2, and ADAR3.

11. The method of any one of embodiments 6-10, wherein the editing agent further comprises: a base editor comprising a polynucleotide programmable RNA binding domain and an adenosine deaminase domain or a cytidine deaminase domain, or a nucleic acid encoding the based editor, wherein the polynucleotide programmable RNA binding domain in conjunction with the targeting ribonucleic acid effects the base alteration.

12. The method of embodiment 11, wherein the polynucleotide programmable RNA binding domain comprises a nuclease inactive variant of Cas13 or a nickase variant of Cas13.

13. The method of embodiment 12, wherein the Cas13 is Cas13a or Cas13b.

14. The method of any one of embodiments 11-13, wherein (i) the cytidine deaminase domain is selected from the group consisting of the apolipoprotein B mRNA-editing enzyme, catalytic polypeptide-like (APOBEC) family of deaminases, such as APOBEC1, APOBEC2, APOBEC3A, APOBEC3B, APOBEC3C, APOBEC3D/E, APOBEC3F, APOBEC3G, APOBEC3H, or APOBEC4; activation-induced cytidine deaminase (AID), such as activation induced cytidine deaminase (AICDA); cytosine deaminase 1 (CDA1) or CDA2; or cytosine deaminase acting on tRNA (CDAT), and (ii) the adenosine deaminase is selected from the group consisting of adenosine deaminase acting on RNA 1 (ADAR1), ADAR2, ADAR3; adenosine deaminase acting on tRNA 1 (ADAT1), ADAT2, ADAT3; and naturally occurring or engineered tRNA-specific adenosine deaminase (TadA).

15. The method of any one of embodiments 6-14, wherein the targeting ribonucleic acid is a guide RNA or a trigger RNA.

16. The method of any of embodiments 11-14, wherein the base editor or the targeting ribonucleic acid is encoded by one or more nucleic acid molecules administered to the subject, preferably the base editor is encoded by one or more RNA molecules, such as one or more mRNA molecules, administered to the subject.

17. The method of embodiment 16, wherein the targeting ribonucleic acid and/or the one or more nucleic acid molecules, such as the one or more mRNA molecules, are administered to the subject with a lipid nanoparticle (LNP), a peptide cage, or a polymer nanoparticle.

18. The method of any one of claims 2-17, wherein the base alteration results in a conversion of an amino acid codon to a premature stop codon, preferably upstream of a splice junction, to thereby down-regulate the expression of the gene.

19. The method of embodiment 18, wherein the base alteration results in the conversion of a CGA, CAG, or TGG codon to a premature TGA, TAG, or TAA stop codon, respectively, and the base editor comprises the cytidine deaminase domain, preferably, the CAG codon is located in proximity to the 5′-end of the gene.

20. The method of any one of embodiments 2-17, wherein the base alteration results in a conversion of a premature stop codon to an amino acid codon to thereby up-regulate the expression of the gene.

21. The method of embodiment 20, wherein the base alteration results in the conversion of a premature UAG, UAA, or UGA stop codon to a CAG, CAA, or CGA, respectively, and the base editor comprises the adenosine deaminase domain, preferably, the premature UAG stop codon is located in proximity to the 5′-end of the gene.

22. The method of any one of the foregoing embodiments, wherein varying amounts of the editing agent, such as varying amounts of the targeting ribonucleic acid are administered to the subject to obtain varying expression levels of the gene.

23. The method of any one of the foregoing embodiments, further comprising administering to the subject the episomal vector comprising the gene.

24. The method of any one of the foregoing embodiments, wherein the episomal vector is a nonviral vector, such as a plasmid, or a viral vector, such as an adeno-associated virus (AAV) vector or an adenovirus vector.

25. The method of embodiment 24, wherein the episomal vector is an AAV vector.

26. The method of any one of the foregoing embodiments, wherein the subject is a human, such as a human subject suffering from a disease selected from the group consisting of Hereditary angioedema, Pompe disease, hemophilia A, hemophilia B, Fabry disease, Huntington disease, Parkinson's disease, Alzheimer's disease, a synucleinopathy, epilepsy, neuropathic pain, wet macular degeneration, Usher 1F, Usher 1B, glaucoma, Leber congenital amaurosis, and Stargardt disease.

### EXAMPLES

Examples are provided below further illustrating different features of the present invention and methodology for practicing the invention. The provided examples do not limit the claimed invention.

### Example 1. In Vivo Administration of Base Editor to Decrease Expression Levels

Mice (Balb/c; Jackson Laboratory) are dosed with 5e10 vg/mouse and 5e9 vg/mouse of recombinant AAV (rAAV) particles expressing human Factor IX (FIX) by intravenous administration to achieve steady state serum levels of FIX expression of approximately 50,000 ng/ml and 5,000 ng/ml, respectively, at 4 weeks post-administration. Expression cassettes are packaged in an AAV viral particle by being encapsidated in an AAV capsid. Viral particles are generally produced using triple transfection protocol.

Week 5 post-administration, a cytosine base editor (CBE) and gRNA targeting FIX exon 1 glutamine codon are administered, which converts CAG codon to a premature UAG stop codon. The CBE mRNA and gRNA are formulated in a single LNP for administration. Mice are separated into 5 groups and administered mRNA/gRNA LNP of 0.25 mpk, 0.5 mpk, 1.0 mpk, 2.0 mpk or 2.0 mpk control mRNA/gRNA LNP. The first plasma sample is collected via serial sampling from mice one day prior to dosing by intravenous administration. In all Groups, samples are collected at 4 hours, 24 hours, 72 hours and 1 week post-dose. A target total of 80 μL of whole blood is drawn via orbital eye bleed. The blood is transferred to lithium heparin tubes and centrifuged at 9,800×g for 10 minutes at 2-5° C. After collection, samples are frozen and stored at ≤−70° C. for further analysis. transgenic hFIX protein in blood plasma measured by ELISA.

### Example 2. In Vivo Administration of Base Editor to Increase Expression Levels

Mice (Balb/c; Jackson Laboratory) are dosed with a non-expressing variant form of recombinant AAV (rAAV) particles expressing human Factor IX (FIX) by intravenous administration. The variant form includes an UAG premature termination codon (PTC) in FIX exon 1. Expression cassettes are packaged in an AAV viral particle by being encapsidated in an AAV capsid. Viral particles are generally produced using triple transfection protocol.

Week 5 post-administration a administer adenine base editor (ABE) and gRNA targeting FIX exon 1 PTC codon are administered, which converts UAG PTC codon to a glutamine CAG codon. The ABE mRNA and gRNA are formulated in a single LNP for administration. Mice are separated into 5 groups and administered mRNA/gRNA LNP of 0.25 mpk, 0.5 mpk, 1.0 mpk, 2.0 mpk or 2.0 mpk control nRNA/gRNA LNP. The first plasma sample is collected via serial sampling from mice one day prior to dosing by intravenous administration. In all Groups, samples are collected at 4 hours, 24 hours, 72 hours and 1 week post-dose. A target total of 80 μL of whole blood is drawn via orbital eye bleed. The blood is transferred to lithium heparin tubes and centrifuged at 9,800×g for 10 minutes at 2-5° C. After collection, samples are frozen and stored at ≤−70° C. for further analysis. transgenic hFIX protein in blood plasma measured by ELISA.

### Example 3. ADAR-Based RNA Editing of AAV-Delivered Transgene

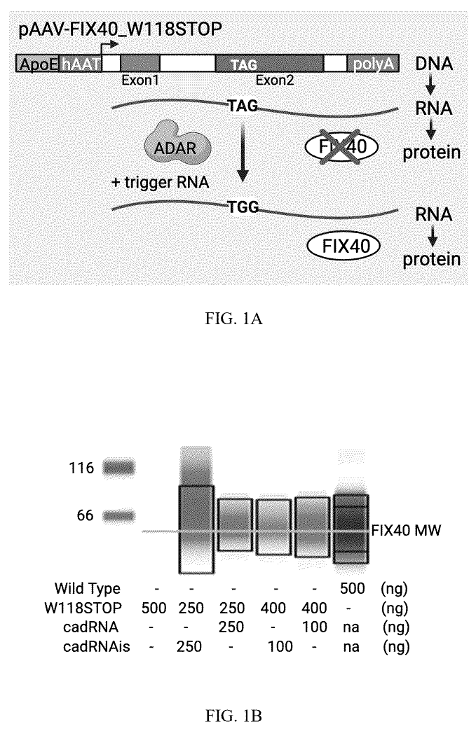

mRNA transcript to edit an early STOP codon allowing for mRNA translation and protein expression of the exogenously delivered transgene. A human factor IX (FIX) (also referred to herein as FIX40) transgene construct (FIX40_W118STOP) was generated with a G to A point mutation in the TGG codon for tryptophan at amino acid position 118 of the FIX sequence (SEQ ID NO: 53), converting it into an early or premature TAG stop codon (FIG. 1A). The point mutation completely abolished FIX40 protein expression as demonstrated by WES™ automated capillary-based immunoassay analysis (ProteinSimple, Bio-Techne) (FIG. 1B) and enzyme-linked immunosorbent assay (ELISA) (FIG. 1C) in Huh7 cell transfection experiments.

The ability of endogenous ADARs to perform an A to I edit on FIX40_W118STOP mRNA was tested in Huh7 cells. Briefly, co-transfection experiments of plasmids carrying the FIX40_W118STOP construct and trigger RNAs yielded FIX expression, as detected by WES (FIG. 1B) and ELISA (FIG. 1C) at 48 hours, reaching between 30-45% of the levels of FIX from a wild type FIX expression construct, depending on FIX construct and trigger RNA concentration. The trigger RNA was driven by pol3 from a human U6 promoter plasmid. Trigger RNAs were 200 nucleotides long and self-circularized, and were either perfectly complementary to the mRNA target (cadRNA; SEQ ID NO: 54) or carried interspersed mismatches (cadRNAis; SEQ ID NO: 55). Not wishing to be bound by any theory, interspered missmatches may contribute to decreased bystander (off-target) editing. Together, these results demonstrate that transgene expression can be turned on by ADAR-based RNA editing in vitro.

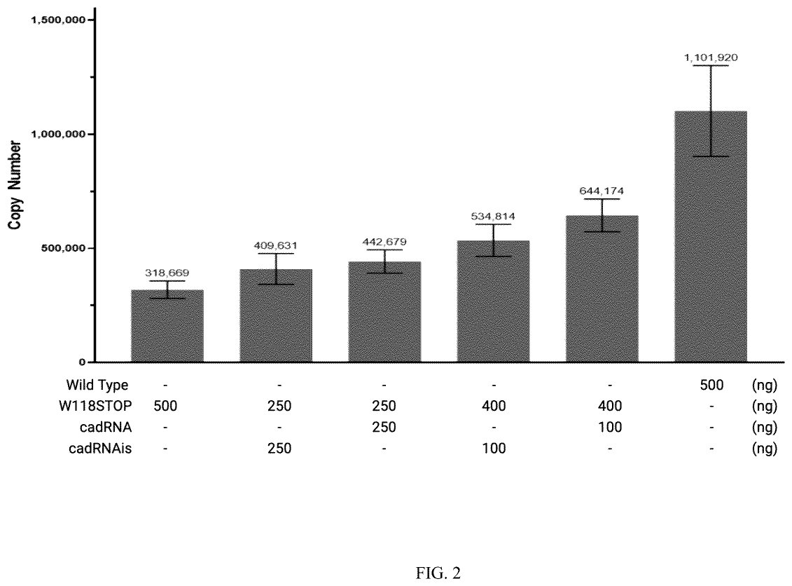

To determine if the introduction of the STOP codon had any effects on the mRNA abundance and to determine the efficiency of the RNA editing event at the molecular level, RNA was extracted from the transfected Huh7 cells. Total FIX40_W118STOP mRNA (FIX40 mRNA) was quantified by qPCR. Quantification of FIX40 mRNA expressed as copy numbers showed that the introduction of the STOP codon caused a ˜35% decrease in the levels of FIX40 mRNA compared to the mRNA from the wild type FIX expression construct (FIG. 2, lane 1 versus lane 6). Not wishing to be bound by any theory, the decrease in FIX40 mRNA levels may be driven by nonsense-mediated decay. Additionally, comparison of protein levels and mRNA abundance of the base-edited samples shows a strong correlation between the measurements (FIG. 1B and FIG. 1C vs. FIG. 2, lanes 2-5) Finally, Sanger sequencing of the mRNA showed that 100% of mRNA molecules had been edited from TAG to TGG at the Wi 18 position (not shown), demonstrating that no bystander edits occurred.

These results agreed with the high correlation observed between protein and mRNA levels.

### Conclusion

The results show that endogenous ADARs can be used to control the expression of an exogenously delivered transgene as an ON switch that can be applied to gene therapies. These in vitro experiments demonstrate that trigger RNA specifically targets ADARs to edit a STOP codon in every mRNA molecule produced from the delivered transgene to allow for protein expression.

### Example 4. Mouse In Vivo Study

To demonstrate the functioning of the ADAR-mediated base editing system in animals an in vivo mouse study was designed using recombinant AAV (rAAV) particles to deliver FIX expression cassette payloads. Mice were dosed intravenously (tail vein) with either AAV-encapsidated FIX40 or AAV-encapsidated FIX40_W118STOP (Table 4). To generate non-limiting levels of FIX40_W 118STOP mRNA, a low and high dose of rAAV were tested.

TABLE 4

| | | | Number | | | | |
|---|---|---|---|---|---|---|---|
| | | | of Male | AAV Vector | Vector | Vector Dose | LNP-cadRNAis |
| Group | Strain | Age | Mice | Payload | Diluent | (vgs/animal) | Dose (mass/kg) |
| 1 | C57BL/6J | 8 Weeks | 12 | FIX40_W118STOP | PBS Pluronic | 5.00E+09 | 2 |
| 2 | C57BL/6J | 8 Weeks | 12 | FIX40_W118STOP | PBS Pluronic | 5.00E+10 | 2 |
| 3 | C57BL/6J | 8 Weeks | 12 | FIX40 | PBS Pluronic | 5.00E+09 | N/A |
| 4 | C57BL/6J | 8 Weeks | 12 | None | PBS Pluronic | N/A | 2 |

Expression of FIX40 in plasma was measured by ELISA at Day 0, Day 7 and Day 14 in animals dosed at 5e9 or 5e10 vg/animal. No FIX40 expression was detected in the FIX40_W118STOP dosed animals (Table 5; BQL: below detection limit). Expression of the control FIX40 construct showed the expected dose-response and time dependent increase from Day 7 to Day 14. Values expressed as average ng/ml for the 3 animals in each group.

TABLE 5

| | | | | | | Plasma hFIX (ng/mL), | | |
|---|---|---|---|---|---|---|---|---|
| | | | Number | AAV Vector | Vector Dose | average per group | | |
| Group | Strain | Age | of Males | Payload | (vgs/animal) | Day 0 | Day 7 | Day 14 |
| 1 | C57BL/6 | 8 Weeks | 3 | FIX40_W118STOP | 5.00E+09 | BQL | BQL | BQL |
| 2 | C57BL/6 | 8 Weeks | 3 | FIX40_W118STOP | 5.00E+10 | BQL | BQL | BQL |
| 3 | C57BL/6 | 8 Weeks | 3 | FIX40 | 5.00E+09 | BQL | 70.4 | 155.5 |
| 4 | C57BL/6 | 8 Weeks | 3 | FIX40 | 5.00E+10 | BQL | 2312.6 | 5179.4 |
| 5 | C57BL/6 | 8 Weeks | 3 | None | N/A | BQL | BQL | BQL |

After establishing the base level of FIX40 expression for each group, trigger RNA is delivered via LNP. Based on the in vitro results, addition of circular trigger RNA is expected to recruit endogenous ADARs to edit the W118STOP codon to a TGG tryptophan codon. FIX40 expression levels in plasma are measured daily for 4 weeks to determine editing efficiency and editing durability. At the end of the study, animals are sacrificed and liver RNA extracted to determine FIX40 RNA levels and editing efficiency.

### Example 5. In Vivo Study to Determine Editing Efficiency and Durability of FIX40W118STOP Induction by ADAR-cadRNAis Complexes

In a follow up study, animals are dosed with rAAV vectors on Day 0 and LNPs on Day 14. FIX40 levels are measured daily to determine the ability of trigger cadRNAis and endogenous or exogenous ADARs to edit FIX40W118STOP mRNA and allow for protein expression.

TABLE 6

| | | | | | | | LNP- | |
|---|---|---|---|---|---|---|---|---|
| | | | Number | | | | cadRNAis | ADAR |
| | | | of Male | | Vector | Vector Dose | Dose (2 | mRNA |
| Group | Strain | Age | Mice | rAAV Payload | Diluent | (vgs/animal) | mg/kg) | (mg/kg) |
| 1 | C57BL/6J | 8 Weeks | 12 | FIX40_W118STOP | PBS | 5.00E+10 | linear | N/A |
| 2 | C57BL/6J | 8 Weeks | 12 | FIX40_W118STOP | PBS | 5.00E+10 | circular | N/A |
| 3 | C57BL/6J | 8 Weeks | 12 | FIX40_W118STOP | PBS | 5.00E+10 | linear | 2 |
| 4 | C57BL/6J | 8 Weeks | 12 | FIX40_W118STOP | PBS | 5.00E+10 | circular | 2 |
| 5 | C57BL/6J | 8 Weeks | 12 | FIX40 | PBS | 5.00E+09 | N/A | N/A |
| 6 | C57BL/6J | 8 Weeks | 12 | None | PBS | N/A | N/A | N/A |

While the invention has been described and illustrated with reference to certain particular embodiments thereof, those skilled in the art will appreciate that various adaptations, changes, modifications, substitutions, deletions, or additions of procedures and protocols may be made without departing from the spirit and scope of the invention.

1. A method of regulating expression of a gene located on an episomal vector in a subject in need thereof, comprising administering to the subject an editing agent that effects an alteration in a region of an mRNA transcript of the gene to thereby regulate the expression of the gene.

2. The method of claim 1, wherein the editing agent effects a base alteration in the region of the mRNA transcript of the gene.

3. The method of claim 1, wherein the alteration in a region of an mRNA transcript of the gene alters the stability of the mRNA transcript, the initiation or level of the translation of the mRNA transcript, the stability and/or activity of the translated protein.

4. The method of claim 2, wherein the region of the mRNA transcript comprises a premature termination codon convertible to an amino acid codon via the base alteration, or an amino acid codon convertible to a premature termination codon via the base alteration.

5. The method of claim 2, wherein the base alteration (a) is within a microRNA targeting site or a toe-hold switch site, or (b) induces a ribosomal frameshift or alters a codon encoding an amino acid residue critical to function and/or structure of an encoded protein.

6. The method of claim 2, wherein the editing agent comprises a targeting ribonucleic acid complementary to the region of the mRNA transcript.

7. The method of claim 6, wherein the targeting ribonucleic acid is linear.

8. The method of claim 6, wherein the targeting ribonucleic acid is circular.

9. The method of claim 6, wherein the targeting ribonucleic acid effects the base alteration via binding to an endogenous adenosine deaminase domain.

10. The method of claim 9, wherein the adenosine deaminase is selected from the group consisting of adenosine deaminase acting on RNA 1 (ADAR1), ADAR2, and ADAR3.

11. The method of claim 6, wherein the editing agent further comprises:
a base editor comprising a polynucleotide programmable RNA binding domain and an adenosine deaminase domain or a cytidine deaminase domain, or a nucleic acid encoding the based editor,
wherein the polynucleotide programmable RNA binding domain in conjunction with the targeting ribonucleic acid effects the base alteration.

12. The method of claim 11, wherein the polynucleotide programmable RNA binding domain comprises a nuclease inactive variant of Cas13 or a nickase variant of Cas13.

13. The method of claim 12, wherein the Cas13 is Cas13a or Cas13b.

14. The method of claim 11, wherein (i) the cytidine deaminase domain is selected from the group consisting of the apolipoprotein B mRNA-editing enzyme, catalytic polypeptide-like (APOBEC) family of deaminases, such as APOBEC1, APOBEC2, APOBEC3A, APOBEC3B, APOBEC3C, APOBEC3D/E, APOBEC3F, APOBEC3G, APOBEC3H, or APOBEC4; activation-induced cytidine deaminase (AID); cytosine deaminase 1 (CDA1) or CDA2; or cytosine deaminase acting on tRNA (CDAT), and (ii) the adenosine deaminase is selected from the group consisting of adenosine deaminase acting on RNA 1 (ADAR1), ADAR2, ADAR3; adenosine deaminase acting on tRNA 1 (ADAT1), ADAT2, ADAT3; and naturally occurring or engineered tRNA-specific adenosine deaminase (TadA).

15. The method of claim 6, wherein the targeting ribonucleic acid is a guide RNA or trigger RNA.

16. The method of claim 11, wherein the base editor or the targeting ribonucleic acid is encoded by one or more nucleic acid molecules administered to the subject.

17. The method of claim 16, wherein the targeting ribonucleic acid and/or the one or more nucleic acid molecules are administered to the subject with a lipid nanoparticle (LNP), a peptide cage, or a polymer nanoparticle.

18. The method of claim 2, wherein the base alteration results in a conversion of an amino acid codon to a premature stop codon to thereby down-regulate the expression of the gene.

19. The method of claim 18, wherein the base alteration results in the conversion of a CGA, CAG, or TGG codon to a premature TGA, TAG, or TAA stop codon, respectively, and the base editor comprises the cytidine deaminase domain.

20. The method of claim 2, wherein the base alteration results in a conversion of a premature stop codon to an amino acid codon to thereby up-regulate the expression of the gene.

21. The method of claim 20, wherein the base alteration results in the conversion of a premature UAG, UAA, or UGA stop codon to a CAG, CAA, or CGA, respectively, and the base editor comprises the adenosine deaminase domain.

22. The method of claim 1, wherein varying amounts of the editing agent, are administered to the subject to obtain varying expression levels of the gene.

23. The method of claim 1, further comprising administering to the subject the episomal vector comprising the gene.

24. The method of claim 1, wherein the episomal vector is a nonviral vector or a viral vector.

25. The method of claim 24, wherein the episomal vector is an AAV vector.

26. The method of claim 1, wherein the subject is a human suffering from a disease selected from the group consisting of Hereditary angioedema, Pompe disease, hemophilia A, hemophilia B, Fabry disease, Huntington disease, Parkinson's disease, Alzheimer's disease, a synucleinopathy, epilepsy, neuropathic pain, wet macular degeneration, Usher 1F, Usher 1B, glaucoma, Leber congenital amaurosis, and Stargardt disease.
